(19) United States
(12) Patent Application Publication
(10) Pub. No.: US 2018/0045737 A1
(43) Pub. Date: Feb. 15, 2018

(54) METHODS FOR DETECTING ADVERSE LOCAL TISSUE REACTION (ALTR) NECROSIS

(71) Applicants: Carl Deirmengian, Newton Square, PA (US); Keith Kardos, Bethlehem, PA (US); James W. Stave, Bear, DE (US); Tony Joaquim, Mount Laurel, NJ (US); Thomas K. Fehring, Charlotte, NC (US)

(72) Inventors: Carl Deirmengian, Newton Square, PA (US); Keith Kardos, Bethlehem, PA (US); James W. Stave, Bear, DE (US); Tony Joaquim, Mount Laurel, NJ (US); Thomas K. Fehring, Charlotte, NC (US)

(21) Appl. No.: 15/664,402

(22) Filed: Jul. 31, 2017

Related U.S. Application Data
(63) Continuation of application No. PCT/US2016/045158, filed on Aug. 2, 2016.
(60) Provisional application No. 62/200,885, filed on Aug. 4, 2015.

(51) Int. Cl. G01N 33/68; C12Q 1/68

(52) U.S. Cl. CPC G01N 33/6893; C12Q 1/6883; G01N 2800/7095; G01N 2800/60; C12Q 2600/106; C12Q 2600/158

(57) ABSTRACT

This invention relates to field of screening and diagnosing adverse local tissue reaction (ALTR) in a subject who has received a joint replacement by measuring the level of a nucleic acid or protein biomarker that are elevated in patients suffering from ALTR, even those with no symptoms. The early diagnosis of the ALTR can lead to its treatment and thus, the prevention of implant failure caused by the ALTR. The elevated proteins and genes are also the basis for treatment for ALTR and provide targets for drug development and basic research.

### CROSS-REFERENCE TO RELATED APPLICATIONS

This application claims priority under 35 U.S.C. §119(e) to U.S. Provisional Application No. 62/200,885, filed Aug. 4, 2015, which is incorporated herein by reference in its entirety.

### BACKGROUND OF THE INVENTION

An adverse local tissue reaction (ALTR) can occur in patients having implants, such as total hip arthroplasty and hip resurfacing arthroplasty. Over time, the metal particles around some implants can cause damage to bone and/or soft tissue surrounding the implant and joint resulting in an ALTR. ALTRs can occur in patients with a metal-on-polyethylene (MOP) bearing as well as with a metal-on-metal (MOM) bearing prosthetics, although MOM bearing prosthetics produce considerably less wear debris than MOP bearing prosthetics. Despite the reduced wear rates, these microparticle debris generated for instance from wear at the articulation, corrosion at the taper junction and abrasion of loose components, produce soluble metal ions that can be measured in the blood. MOM hip articulations were reintroduced into the market based upon the promise of decreased wear rates, increased longevity of the prosthetic, and reduction of dislocation rates as these designs allow for a potential solution for wear-related failures in total hip replacements and for larger diameter femoral heads which have been shown to improve stability and reduce dislocation rates.

Blood ion levels, which represent a balance between ion production from the implant and renal excretion, can vary based on changes in activity levels as well as renal function. Well-functioning MOM implants have shown an increase in serum cobalt and chromium (CoCr) ion levels in blood. A medical device alert from the British Orthopedic Association concerning MOM implants chose cobalt and chromium ion levels of 7 ppb as a threshold for concern. The cytotoxicity of metal debris and prolonged systemic exposure to elevated metal ion levels is not clearly understood. Patients have variable hypersensitivity responses to metallic debris and may also have variable metal ion exposure threshold levels that lead to ALTR. The acidic environment found with crevice corrosion at the head-neck taper may be a factor in addition to the abrasive wear of a CoCr bearing. Therefore, increased bearing wear coupled with increased wear and ion release from the taper may result in more cytotoxic debris and increased blood serum ion levels.

The current evidence suggests that measuring ion levels in blood as a measure of ALTR in patients with MOM implants is unreliable and that increasing ion levels do not correlate with tissue damage. Fehring et al., 2015, Journal of Arthroplasty, 30:107-109. The clinical evaluation and treatment of patients presenting with symptomatic or asymptomatic MOM implants is difficult. Lombardi et al. (J Bone Joint Surg Br., 2012, 94(11 Suppl A):14-8) identified seven clinical scenarios of presentation and a complex algorithm to guide medical decision-making. Key factors within this decision tree include implant track record, radiographic implant position, radiographic evidence of osteolysis, and whether the metal ion levels in the blood are above or below 7 ppb.

In addition to measuring blood and synovial fluid metal ion levels, ultrasound and MRI with metal artifact reduction sequences (MARS) have been used to assess periarticular reactions secondary to metal wear debris. Despite metal reduction software, these scans are frequently difficult to interpret. While each of these tests has merit, at the present time there is no single diagnostic test available which delineates the key issue that demands urgent surgical intervention, i.e., tissue necrosis. Furthermore, despite its excellent track record, complications of MOM implants occur at relatively low rates and include infection and ALTR Tissue destruction in severe ALTR can be so extensive as to make revision difficult. The histological observations associated with ALTR include infiltrations of macrophages and lymphocytes, tissue necrosis, osteolysis, giant cells, granulomas, and pseudotumors.

In contrast, periprosthetic joint infection (PJI) is mediated almost exclusively by increased concentrations of neutrophils which are the body's primary response to invading microorganisms. Alpha defensin (AD) is an antimicrobial protein produced by neutrophils and thus an elevated concentration of AD is a biomarker of PJI. PJI is a single disease process, i.e., infection. In contrast, ALTRs are manifested through multiple disease mechanisms that are mediated by macrophages and lymphocytes. Thus, the biomarkers of ALTR are anticipated to be different than PJI and reflective of large numbers of macrophages and lymphocytes rather than neutrophils.

The identification of biomarkers that permit early diagnosis, monitoring, and differentiation of ALTR and PJI is of great value to the orthopedic community.

Therefore there is a need in the art for a reliable test to guide surgeons and patients in the shared decision-making process of when therapeutic intervention is necessary to prevent disabling tissue damage. The present invention addresses this need.

### BRIEF SUMMARY OF THE INVENTION

The invention provides a method of treating an adverse local tissue reaction (ALTR) in a test subject having an implant, the method comprising:
a. requesting a test to determine whether the test subject has at least one biomarker of ALTR in a bodily fluid sample obtained from the test subject;
b. comparing the levels of the at least one biomarker in the test subject's bodily fluid sample with a control level, wherein a difference in the level of the at least one biomarker in the test subject's bodily fluid sample as compared with the control level is indicative of an ALTR in the test subject; and,
c. wherein when ALTR is detected, the test subject undergoes therapeutic intervention.

The invention further provides a method for diagnosing and treating ALTR in a test subject with an implant, the method comprising:
a. analyzing a test subject's bodily fluid sample for the presence or absence of at least one biomarker, wherein if the at least one biomarker is detected the test subject is diagnosed with ALTR; and,
b. performing diagnosed therapeutic intervention on the diagnosed test subject.

In certain embodiments of the methods of the invention, the biomarker is at least one selected from the group consisting of Neutrophil defensin 1, C-reactive protein, Growth-regulated alpha protein, Neutrophil elastase, Interleukin 1-alpha, Interleukin 6, Interleukin 8, Interleukin 12-beta, Interleukin 15, C-X-C motif chemokine 10, Lactate, Leptin, Monocyte chemotactic protein 1, Monocyte chemotactic protein 3, C-C motif chemokine 22, Tumor necrosis factor receptor superfamily member 11B, Osteopontin; Platelet-derived growth factor subunit B, Pentraxin-3, Tumor necrosis factor alpha, Vascular endothelial growth factor, Tumor necrosis factor ligand superfamily member 6 and Soluble intercellular adhesion molecule-1.

In other embodiments, the at least one biomarker is selected from the group consisting of Interleukin 15, Platelet-derived growth factor subunit B, Osteopontin, Tumor necrosis factor ligand superfamily member 6 and Soluble intercellular adhesion molecule-1. In yet other embodiments, the at least one biomarker is selected from the group consisting of Interleukin 15, Platelet-derived growth factor subunit B, and Osteopontin.

In other embodiments, the at least one biomarker is selected from the group consisting of Interleukin 8, C-reactive protein, Interleukin 12-beta, Interleukin 15, Monocyte chemotactic protein 1, Monocyte chemotactic protein 3, Pentraxin-3 and Tumor necrosis factor ligand superfamily member 6.

The invention further provides a method of diagnosing ALTR in a test subject with an implant, the method comprising:
a. assessing whether or not T-cells are present at the implant site of the test subject by assessing for the presence of at least one biomarker of T-cell activity selected from the group consisting of Interleukin 15 and Tumor necrosis factor ligand superfamily member 6 in a bodily fluid sample obtained from the implant site, wherein if the least one biomarker is detected in the sample, the test subject is diagnosed with ALTR; and,
b. recommending a therapeutic intervention for the test subject.

The invention further provides a method for diagnosing ALTR in a test subject with an implant, the method comprising:
a. assessing whether or not macrophages are present at the implant site of the test subject by assessing for the presence of at least one biomarker of macrophages selected from the group consisting of Monocyte chemotactic protein 1 and Monocyte chemotactic protein 3 in a bodily fluid sample obtained from the implant site, wherein if the least one biomarker is detected in the sample, the test subject is diagnosed with ALTR; and,
b. recommending a therapeutic intervention for the test subject.

The invention further provides a method for diagnosing ALTR in a test subject with an implant, the method comprising:
a. analyzing the presence of bone growth at the implant site of the test subject by measuring the level of at least one biomarker of bone growth selected from the group consisting of Osteopontin and Platelet-derived growth factor subunit B in a bodily fluid sample obtained from the implant site, wherein if the least one biomarker is detected in the sample, the test subject is diagnosed with ALTR; and,
b. recommending a therapeutic intervention for the test subject.

The invention further provides a method for diagnosing ALTR in a test subject with an implant, the method comprising:
a. analyzing a bodily fluid sample from the implant site of the test subject for the presence of a local inflammatory response by measuring the level of at least one biomarker comprising Pentraxin-3;
b. comparing the levels of Pentraxin-3 in the test subject's bodily fluid sample with a control level, wherein when an increase in the level of Pentraxin-3 in the bodily fluid sample from the implant site is detected as compared with a control level, the test subject is diagnosed with ALTR; and,
c. recommending a therapeutic intervention for the test subject.

The invention further provides a method for diagnosing ALTR in a test subject with an implant, the method comprising:
a. analyzing a bodily fluid sample from the implant site of the test subject for the presence of a systemic inflammatory response by measuring the level of at least one biomarker comprising C-reactive protein;
b. comparing the levels of C-reactive protein in the test subject's bodily fluid sample with a control level, wherein when a decrease or a similar level of C-reactive protein in the bodily fluid sample from the implant site is detected as compared with a control level and the subject does not have elevated biomarkers indicative of infection, the test subject is diagnosed with ALTR; and,
c. recommending a therapeutic intervention for the test subject.

The invention further provides a method for diagnosing ALTR in a test subject with an implant, the method comprising:
a. analyzing a test subject's bodily fluid sample for the presence of a biomarker by using a monoclonal antibody specific for the biomarker, wherein presence of the biomarker creates a biomarker-antibody complex, which complex is detected using a detection agent;
b. providing a diagnosis of ALTR in the test subject when the detection agent is detected; and,
c. providing recommendation for a therapeutic intervention for the test subject.

The invention further provides a method of distinguishing between ALTR and periprosthetic joint infection (PJI) in a test subject having an implant, the method comprising:
a. requesting a test to determine whether the test subject has at least one biomarker of ALTR or PJI in a bodily fluid sample obtained from a joint in the test subject;
b. comparing the levels of the at least one biomarker in the test subject's bodily fluid sample with a control level, wherein a difference in the level of the at least one biomarker in the test subject's bodily fluid sample as compared with the control level is an indication that the test subject has at least one of an ALTR and PJI; and,
c. wherein when the distinction between ALTR and PJI is indicated, a therapeutic intervention, appropriate for the condition of the diagnosed test subject condition, is recommended.

The invention further provides a method of distinguishing between ALTR and PJI in a test subject having an implant, the method comprising:
a. requesting a test to determine whether the test subject has at least one biomarker of ALTR or PJI in a bodily fluid sample obtained from a joint in the test subject;
b. analyzing using an algorithm for the presence or a level of the at least one biomarker in the test subject's bodily fluid sample, wherein the algorithm facilitates differentiation between an ALTR and PJI in the test subject;
c. requesting further analysis for testing using additional biomarkers using the algorithm to confirm (b); and,
d. wherein when a distinction between ALTR and PJI is indicated, a therapeutic intervention for the diagnosed test subject is recommended.

In certain embodiments the at least one biomarker comprises one or more of IL-6, CRP, PDGF or OPN. In other embodiments the at least one biomarker comprises IL-8 and/or OPN. In certain embodiments the additional biomarker comprises PDGF. In various embodiments, PDGF AB/BB.

In certain embodiments of any of the methods of the invention, the therapeutic intervention is a revision surgery.

In certain embodiments of any of the methods of the invention, the bodily fluid sample comprises at least one selected from the group consisting of blood, serum and synovial fluid.

In certain embodiments of any of the methods of the invention, the implant is a prosthesis.

In certain embodiments of any of the methods of the invention, the implant is at least one selected from the group consisting of a hip, a knee, a shoulder, an ankle and a wrist.

In certain embodiments of any of the methods of the invention, the ALTR is at least one condition selected from the group consisting of hypersensitivity, metal hypersensitivity and tissue necrosis.

In certain embodiments of any of the methods of the invention, the indication or diagnosis of ALTR in a test subject with an implant is provided with a sensitivity of at least 45% and a specificity of at least 900/%.

In certain embodiments of any of the methods of the invention, the subject is a human.

The invention further provides a kit comprising an antibody or an oligonucleotide probe set against at least one biomarker selected from the group consisting of Neutrophil defensin 1, C-reactive protein, Growth-regulated alpha protein, Neutrophil elastase, Interleukin 1-alpha, Interleukin 6, Interleukin 8, Interleukin 12-beta, Interleukin 15, C-X-C motif chemokine 10, Lactate, Leptin, Monocyte chemotactic protein 1, Monocyte chemotactic protein 3, C-C motif chemokine 22, Tumor necrosis factor receptor superfamily member 11B, Osteopontin; Platelet-derived growth factor subunit B, Pentraxin-3, Tumor necrosis factor alpha, Vascular endothelial growth factor, Tumor necrosis factor ligand superfamily member 6 and Soluble intercellular adhesion molecule-1, and instructions for use thereof, wherein the instructions comprise:
a. measuring the level of the biomarker in a bodily fluid sample from a test subject;
b. providing indication on presence or absence of an ALTR or PJI; and,
c. providing recommendation of whether or not the subject should undergo a therapeutic intervention.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-IC are a set of tables depicting assays suitable for use in the primary screening of synovial fluid samples for 99 biomarkers of adverse local tissue reaction (ALTR).

FIG. 2 is a table depicting information regarding the individual bodily fluid samples used in the primary screening for biomarkers of ALTR.

FIG. 3 is a table listing certain biomarkers selected for the secondary screening of synovial fluid samples.

FIGS. 4A(Part 1/8) through 4A(Part 8/8), and 4B are a series of tables showing the primary screening results. FIG. 4A(Part 1/8) through 4A(Part 8/8): Results for 99 unique biomarkers in pooled control samples, such as a pool of osteoarthritis (OA), a pool of aseptic, and a pool of periprosthetic joint infection (PJI) synovial fluid samples, and 5 test samples, which are individual metal on metal (MOM) synovial fluid samples. FIG. 4B: Primary screening data for pentraxin3. The analysis was done using a Luminex immunoassay kit from Biorad (Cat#171BL033M). Units are pg/ml unless indicated otherwise.

FIGS. 5A-5C are a set of tables depicting the characteristics of MOM samples analyzed in the secondary biomarker validation assay.

FIGS. 6A(Part1/4) through 6A(Part 4/4), and 6B(Part1/4) through 6B(Part 4/4) are a series of tables summarizing the results of the secondary analysis which involved many fewer biomarkers (23) and much larger numbers of samples (68 individual MOM, aseptic, OA and PJI samples). FIG. 6A(Part1/4) through 6A(Part 4/4): Listing of biomarkers 1-11. FIG. 6B(Part 1/4) through 6B(Part 4/4): Listing of biomarkers 12-23. In this secondary analysis, IL-6 assay was performed twice using 2 different kits. The cutoff concentration values between groups as well as the clinical sensitivity and specificity were established by a Receiver Operating Characteristic (ROC) curve analysis of the data. The positive responses relative to cutoffs are in red. S/CO-signal to cutoff. Units are pg/ml unless otherwise indicated.

FIGS. 7 A-7P are a series of dot plots displaying analysis of biomarkers in aseptic, MOM, OA, and PJI samples with ROC cut-offs indicated by the dashed-lines.

FIG. 8 is a table listing the cutoff concentration values as well as the clinical sensitivity and specificity for 16 unique MOM biomarkers. These values were established by a Receiver Operating Characteristic (ROC) curve analysis using either aseptic samples or all controls (aseptic, OA and PJI). AUC denotes area under the curve.

FIGS. 9A-9H are a series of dot plots displaying ROC analysis of PDGFB and IL-15 biomarkers in MOM samples.

FIGS. 10A-10D are a series of dot plots displaying ROC analysis of PDGFB and IL-15 biomarkers in aseptic versus MOM samples and in all controls versus MOM samples.

FIGS. 11A-11C are a series of dot plots displaying ROC analysis of IL-6(1) and IL-8 biomarkers in aseptic versus MOM samples and in all controls versus MOM samples.

FIG. 12 is a table depicting the sensitivity and specificity of the biomarker C-reactive protein (CRP) for diagnosing MOM samples at different cutoffs.

FIG. 13 is a table depicting the sensitivity and specificity of biomarkers comprised of multiple proteins for diagnosis of ALTR

FIG. 14 is a dot plot displaying IL-8 biomarker in ALTR, MOP, aseptic and PJI samples, with ROC cut-offs indicated by the dashed-lines.

FIG. 15 is a series of dot plots displaying IL-6, CRP, PDGF and OPN biomarkers in ALTR, MOP, aseptic and PJI samples with ROC cut-offs indicated by the dashed-lines. These biomarkers may be used to differentiate ALTR and PJI.

FIG. 16 is a scatter plot displaying IL-8 and OPN as a combination of biomarkers for ALTR and PJI in ALTR, aseptic, MOP and PJI samples with each respective ROC cut-off indicated by the dashed-line.

FIG. 17 summarizes the response of IL-8 and OPN biomarkers in ALTR, aseptic, MOP, PJI, rheumatoid arthritis (RA) and trauma/injury samples. IL-8 and OPN may be used to screen for ALTR

FIG. 18 summarizes an ALTR confirmatory test and includes a dot plot displaying ROC analysis of PDGF biomarker in ALTR, aseptic, MOP and PJI samples.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “adverse local tissue reaction” or “ALTR” as used herein, means adverse events related to implants including but not limited to macroscopic staining of soft-tissues associated with abnormal wear (Metallosis), macrophage infiltration (Innate Immunity), Aseptic Lymphocyte-Dominated Vasculitis Associated-Lesion (ALVAL), periprosthetic osteolysis/aseptic loosening, tissue necrosis and some forms of hypersensitivity. The term “adverse reaction to metal debris (ARMD)” is an ALTR that refers to periprosthetic local soft tissue and/or bone inflammation and tissue injury, comprising an inflammatory cell infiltrate, with or without extensive soft tissue necrosis, and vascular changes.

As used herein the terms “alteration,” “defect,” “variation,” or “mutation,” refers to a mutation in a gene in a cell that affects the function, activity, expression (transcription or translation) or conformation of the polypeptide that it encodes. Mutations encompassed by the present invention can be any mutation of a gene in a cell that results in the enhancement or disruption of the function, activity, expression or conformation of the encoded polypeptide, including the complete absence of expression of the encoded protein and can include, for example, missense and nonsense mutations, insertions, deletions, frameshifts and premature terminations. Without being so limited, mutations encompassed by the present invention may alter splicing the mRNA (splice site mutation) or cause a shift in the reading frame (frameshift).

The term “amplification” refers to the operation by which the number of copies of a target nucleotide sequence present in a sample is multiplied.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)2, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA, 85:5879-5883; Bird et al., 1988, Science, 242:423-426).

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations, K and A, light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “applicator,” as the term is used herein, is meant any device including, but not limited to, a hypodermic syringe, a pipette, an iontophoresis device, a patch, and the like, for administering the compositions of the invention to a subject.

“Aggregation” means a massing together or clustering of independent but similar units, such as proteins, particles, parts, or bodies.

The term “antigen” or “Ag” as used herein is defined as a molecule that binds to a receptor of the immune system and provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA A skilled artisan will understand that any DNA, which comprises a nucleotide sequence or a partial nucleotide sequence encoding a protein that elicits an immune response, therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “auto-antigen” means, in accordance with the present invention, any self-antigen which is mistakenly recognized by the immune system as being foreign. Auto-antigens comprise, but are not limited to, cellular proteins, phosphoproteins, cellular surface proteins, cellular lipids, nucleic acids, glycoproteins, including cell surface receptors.

“Biological sample” or “sample” as used herein means a biological material isolated from an individual. The biological sample may contain any biological material suitable for detecting the desired biomarkers, and may comprise cellular and/or non-cellular material obtained from the individual. A biological sample may be of any biological tissue or fluid. Frequently the sample will be a “clinical sample” which is a sample derived from a patient. Typical clinical samples include, but are not limited to, bodily fluid samples such as synovial fluid, sputum, blood, urine, blood plasma, blood serum, sweat, mucous, saliva, lymph, bronchial aspirates, peritoneal fluid, cerebrospinal fluid, and pleural fluid, and tissues samples such as blood-cells (e.g., white cells), tissue or fine needle biopsy samples and abscesses or cells therefrom. Biological samples may also include sections of tissues, such as frozen sections or formalin fixed sections taken for histological purposes.

As used herein, “biomarker” in the context of the present invention encompasses, without limitation, proteins, nucleic acids, and metabolites, together with their polymorphisms, mutations, variants, modifications, subunits, fragments, protein-ligand complexes, and degradation products, protein-ligand complexes, elements, related metabolites, and other analytes or sample-derived measures. Biomarkers can also include mutated proteins or mutated nucleic acids. Biomarkers also encompass non-blood borne factors or non-analyte physiological markers of health status, such as clinical parameters, as well as traditional laboratory risk factors. As defined by the Food and Drug Administration (FDA), a biomarker is a characteristic (e.g. measurable DNA and/or RNA) that is “objectively measured and evaluated as an indicator of normal biologic processes, pathogenic processes, or pharmacologic responses to a therapeutic intervention or other interventions”. Biomarkers also include any calculated indices created mathematically or combinations of any one or more of the foregoing measurements, including temporal trends and differences.

As used herein, a “biosensor” is an analytical device for the detection of an analyte in a sample. Biosensors can comprise a recognition element, which can recognize or capture a specific analyte, and a transducer, which transmits the presence or absence of an analyte into a detectable signal.

As used herein, the term “data” in relation to one or more biomarkers, or the term “biomarker data” generally refers to data reflective of the absolute and/or relative abundance (level) of a product of a biomarker in a sample. As used herein, the term “dataset” in relation to one or more biomarkers refers to a set of data representing levels of each of one or more biomarker products of a panel of biomarkers in a reference population of subjects. A dataset can be used to generate a formula/classifier of the invention. According to one embodiment, the dataset need not comprise data for each biomarker product of the panel for each individual of the reference population. For example, the “dataset” when used in the context of a dataset to be applied to a formula can refer to data representing levels of products of each biomarker for each individual in one or more reference populations, but as would be understood can also refer to data representing levels of each biomarker for 99%, 95%, 90%, 85%, 80%, 75%, 70%0/or less of the individuals in each of said one or more reference populations and can still be useful for purposes of applying to a formula.

The term “control level” as used herein means a biomarker level in a sample from a subject where the subject does not have the condition being tested. The term “control level” is also construed herein to mean an average level of an endogenous biomarker in samples obtained from more than one subject where the subject does not have the condition being tested. Thus, as used herein, the term “endogenous biomarker” relates to naturally-occurring levels of a biomarker in a control sample such as in a control/normal/healthy individual. The term “control level” is also construed herein to mean a reference biomarker level obtained through calculation of what such a biomarker level might be in samples from a hypothetical group of subjects not having the condition being tested. A “control level” should also be construed herein to mean a level of biomarker in, for example, an infected prosthetic joint (periprosthetic joint infection, PJI) when the control level in this context is compared to a biomarker level in the test subject having, for example, ALTR from a MOM joint implant. The control level is therefore simply a level of biomarker against which a test level is measured. Examples of control biomarkers who's levels can be measured include, without limitation, biomarkers that can be measured in any bodily fluid sample, where the sample includes, without limitation, a bodily fluid sample from a joint of a subject where the subject does not have an implant, for example, the subject has not undergone a joint replacement (a native joint), a bodily fluid from a joint where the subject has an implant, i.e., a prosthetic joint but where the joint is not infected (an aseptic joint), a bodily fluid from a joint where the subject has an implant, i.e., a prosthetic joint, where the joint is infected (a septic joint or PJI), and the like. The control biomarker level thus serves as a comparator against which a test sample can be compared.

As used herein, a “detector molecule” is a molecule that may be used to detect a compound of interest. Non-limiting examples of a detector molecule are molecules that bind specifically to a compound of interest, such as, but not limited to, an antibody, a cognate receptor, and a small molecule.

By the phrase “determining the level of marker (or biomarker) expression” is meant an assessment of the degree of expression of a marker in a sample at the nucleic acid or protein level, using technology available to the skilled artisan to detect a sufficient portion of any marker.

“Differentially increased expression” or “up regulation” refers to biomarker levels which are at least 106 or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments therebetween than a control.

“Differentially decreased expression” or “down regulation” refers to biomarker product levels which are at least 10/6 or more, for example, 20%, 30%, 40%, or 50%, 60%, 70%, 80%, 90% lower or less, and/or 2.0 fold, 1.8 fold, 1.6 fold, 1.4 fold, 1.2 fold, 1.1 fold or less lower, and any and all whole or partial increments therebetween than a control.

A “disease” is a state of health of an animal wherein the animal cannot maintain homeostasis, and wherein if the disease is not ameliorated then the animal's health continues to deteriorate.

As used herein the terms “hypersensitivity” or “hypersensitivity reaction” relate to a series of immune reactions produced by the normal immune system, including allergies and autoimmunity. These reactions may be detrimental in the host by being damaging, uncomfortable, or occasionally fatal. Hypersensitivity reactions require a pre-sensitized (immune) state of the host. Hypersensitivity can be triggered by the presence of an implant (e.g. metal hypersensitivity) and are defined as an immune reaction that is triggered by specific cells of the body's immune system in response to certain implants, particularly metals (for example, nickel, cobalt, and chromium). While metal hypersensitivity can be considered a type of allergy, it does not induce the immediate allergy symptoms that occur when exposed to seasonal or household allergens like pollen, animal dander, mold, etc. Metal hypersensitivity has a delayed onset from the time of exposure to the materials and is not caused by specific antibodies or histamine release that lead to the classical indications of a common allergy (for example, itching, watery eyes, or sneezing). Metal hypersensitivity requires a first-step sensitization stage where T cells recognize, activate, proliferate and form immunological memory upon contact with sensitizing agents like metals. Once immunological memory has been formed, a secondary exposure to metal leads to all the classical inflammatory symptoms of delayed type hypersensitivity, as compared with immediate type hypersensitivity (usually from a food allergy or bee sting). General metal hypersensitivity symptoms caused by metal implant devices could be pain, swelling, loss of range and motion in the affected joint, effusions from the joint, inflammation and premature osteolysis (bone loss) around the metal device.

As used herein, an “immunoassay” refers to a biochemical test that measures the presence or concentration of a substance in a sample, such as a biological sample, using the reaction of an antibody to its cognate antigen, for example the specific binding of an antibody to a protein. Both the presence of the antigen or the amount of the antigen present can be measured.

As used herein, the term “implant” refers to any material inserted or grafted into the body that maintains support and tissue contour including, but not limited to prosthetic joints, screws and plates.

As used herein, an “instructional material” includes a publication, a recording, a diagram, or any other medium of expression which can be used to communicate the usefulness of a component of the invention in a kit for detecting biomarkers disclosed herein. The instructional material of the kit of the invention can, for example, be affixed to a container which contains the component of the invention or be shipped together with a container which contains the component. Alternatively, the instructional material can be shipped separately from the container with the intention that the instructional material and the component be used cooperatively by the recipient.

The term “label” when used herein refers to a detectable compound or composition that is conjugated directly or indirectly to a probe to generate a “labeled” probe. A label may be a component of an assay and may be detectable by itself (e.g. radioisotope labels, fluorescent labels or colloidal gold) or, in the case of an enzymatic label, may catalyze chemical alteration of a substrate compound or composition that is detectable (e.g., Horseradish peroxidase-Tetramethylbenzidine, HRP-TMB). In some instances, primers can be labeled to detect a PCR product. The term “tag” is also used interchangeably with the term “label”.

The “level” of one or more biomarkers means the absolute or relative amount or concentration of the biomarker in the sample.

The terms “biomarker” or “marker,” as used herein, refers to a molecule that can be detected. Therefore, a biomarker according to the present invention includes, but is not limited to, a nucleic acid, a polypeptide, a carbohydrate, a lipid, an inorganic molecule, an organic molecule, each of which may vary widely in size and properties. A “biomarker” can be a bodily substance relating to a bodily condition or disease. A “biomarker” can be detected using any means known in the art or by a previously unknown means that only becomes apparent upon consideration of the marker by the skilled artisan.

The term “biomarker (or marker) expression” as used herein, encompasses the transcription, translation, post-translation modification, and phenotypic manifestation of a gene, including all aspects of the transformation of information encoded in a gene into RNA or protein. By way of non-limiting example, marker expression includes transcription into messenger RNA (mRNA) and translation into protein, as well as transcription into types of RNA such as transfer RNA (tRNA) and ribosomal RNA (rRNA) that are not translated into protein.

The terms “microarray” and “array” refer broadly to “DNA microarrays” (or “DNA chip(s)”), “RNA microarrays”, “protein microarrays”, and “antibody arrays” encompass all art-recognized solid supports, and all art-recognized methods for affixing nucleic acid molecules thereto or for synthesis of nucleic acids thereon and antibodies. Preferred arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 5,800,992, 6,040,193, 5,424,186 and Fodor et al., 1991, Science, 251:767-777, each of which is incorporated by reference in its entirety for all purposes. Arrays may be used to assess large amounts of biological material using high-throughput screening miniaturized multiplexed and parallel processing and detection methods. Arrays may generally be produced using a variety of techniques, such as mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. Nos. 5,384,261, and 6,040,193, which are incorporated herein by reference in their entirety for all purposes. Although a planar array surface is preferred, the array may be fabricated on a surface of virtually any shape or even a multiplicity of surfaces. Arrays may be nucleic acids or antibodies on beads, gels, polymeric surfaces, and fibers such as fiber optics, glass or any other appropriate substrate. (See U.S. Pat. Nos. 5,770,358, 5,789,162, 5,708,153, 6,040,193 and 5,800,992, which are hereby incorporated by reference in their entirety for all purposes.) Arrays may be packaged in such a manner as to allow for diagnostic use or can be an all-inclusive device; e.g., U.S. Pat. Nos. 5,856,174 and 5,922,591 incorporated in their entirety by reference for all purposes. Arrays are commercially available from, for example, Affymetrix (Santa Clara, Calif.) and Applied Biosystems (Foster City, Calif.), and are directed to a variety of purposes, including genotyping, diagnostics, mutation analysis, marker expression, and gene expression monitoring for a variety of eukaryotic and prokaryotic organisms. The number of probes on a solid support may be varied by changing the size of the individual features. In one embodiment the feature size is 20 by 25 microns square, in other embodiments features may be, for example, 8 by 8, 5 by 5 or 3 by 3 microns square, resulting in about 2,600,000, 6,600,000 or 18,000,000 individual probe features.

“Measuring” or “measurement,” or alternatively “detecting” or “detection,” means determining the presence, absence, quantity or amount (which can be an effective amount) of either a given substance within a clinical or subject-derived sample, including the derivation of qualitative or quantitative concentration levels of such substances, or otherwise determining the values or categorization of a subject's clinical parameters.

The terms “metal-on-metal” or “MOM” are used interchangeably herein and refer to a type of implant for joint replacement (e.g. total hip replacement or hip resurfacing arthroplasty) which may containing a metal stem, neck, head, liner, and shell (bearing surface). In some instances a patient with a MOM symptom refers to a patient with a symptomatic/painful joint implant.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

“Polypeptide,” as used herein refers to a polymer in which the monomers are amino acid residues which are joined together through amide bonds. When the amino acids are alpha-amino acids, either the L-optical isomer or the D-optical isomer can be used, the L-isomers being preferred. The terms “polypeptide” or “protein” or “peptide” as used herein are intended to encompass any amino acid sequence and include modified sequences such as glycoproteins. The term “polypeptide” or “protein” or “peptide” is specifically intended to cover naturally occurring proteins, as well as those which are recombinantly or synthetically produced. It should be noted that the term “polypeptide” or “protein” includes naturally occurring modified forms of the proteins, such as glycosylated forms.

A “reference level” of a biomarker means a level of the biomarker that is indicative of a particular disease state, phenotype, or lack thereof, as well as combinations of disease states, phenotypes, or lack thereof. A “positive” reference level of a biomarker means a level that is indicative of a particular disease state or phenotype. A “negative” reference level of a biomarker means a level that is indicative of a lack of a particular disease state or phenotype.

The term “solid support,” “support,” and “substrate” as used herein are used interchangeably and refer to a material or group of materials having a rigid or semi-rigid surface or surfaces. In one embodiment, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, microplates, or other geometric configurations. See U.S. Pat. No. 5,744,305 for exemplary substrates.

The term “therapeutic intervention” as used herein, means a treatment of a patient designed to alleviate a symptom experienced by the patient. The term should be construed to include surgical intervention.

The term “surgical intervention” as used herein, means performing surgery on a subject to remove or replace an implant, such as, to remove or replace a plate or a screw, or to perform a surgical revision of a prosthetic joint.

The terms “total hip replacement” or “THR” as used herein mean the implantation of an implant or device in a subject to replace an existing diseased or injured hip.

As used herein, the term “wild-type” refers to a gene or gene product isolated from a naturally occurring source. A wild-type gene is that which is most frequently observed in a population and is thus arbitrarily designated the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to methods for diagnosing and/or treating adverse local tissue reaction (ALTR), in a test subject. The method includes monitoring the presence or absence of a biomarker in a joint bodily fluid or other bodily fluid samples, as well as determining the level of the biomarker in the joint bodily fluid or other bodily fluid. The invention further includes methods that distinguish between an ALTR and a PJI in a test subject.

### Methods of the Invention

The invention provides a series of methods for diagnosing and/or treating an ALTR, in a test subject having an implant (e.g. a prosthesis such as MOM joint replacement).

In one embodiment, the method of the invention comprises requesting a test to determine whether the test subject has at least one biomarker in a bodily fluid sample obtained from the implant site of the test subject. The test may distinguish between the presence or absence of the biomarker in the test sample, or it may identify a level of a biomarker in the test sample that differs from a control level of a biomarker as that term is defined herein. The test sample is preferably from a joint of the test subject, i.e., synovial fluid, but may also be any bodily fluid, for example, blood, plasma or serum. The level of the at least one biomarker from the test subject is assessed and compared to a control level, wherein the presence, absence, increase or decrease in the level of the biomarker in the test subject's bodily fluid sample as compared with the control level is indicative of an ALTR in the test subject. When such presence, increase or decrease is detected, therapeutic intervention (e.g. a revision surgery) is recommended for the test subject. In one embodiment, the invention provides a method for diagnosing ALTR in a test subject having an implant (such as for example, a metal prosthesis). The method of the invention comprises analyzing a test subject's bodily fluid sample for the presence of a biomarker using a polyclonal (such as rabbit) and/or a monoclonal antibody specific for the biomarker, wherein presence of the biomarker creates a biomarker-antibody complex, which complex is detected using a detection agent. When the detection agent is detected, a diagnosis of ALTR in the test subject is made, and treatment is recommended for the patient that may include surgical intervention.

In one embodiment, the invention provides a method for diagnosing ALTR hypersensitivity, or tissue necrosis in a test subject having an implant by assessing whether or not T-cells are present at the implant site. The method of the invention comprises assessing for the presence of at least one biomarker of T-cell activity selected from the group consisting of Interleukin 15 and Tumor necrosis factor ligand superfamily member 6 in a bodily fluid sample obtained from the implant site. If the at least one biomarker is detected in the sample, the test subject is diagnosed with ALTR, whereupon therapeutic intervention is recommended that may or may not include surgical intervention.

In one embodiment, the invention provides a method for diagnosing ALTR in a test subject with an implant by assessing whether or not macrophages are present at the implant site. The method of the invention comprises assessing for the presence of at least one biomarker of macrophages selected from the group consisting of Monocyte chemotactic protein 1, Monocyte chemotactic protein 3, and Macrophage inflammatory protein 1-alpha in a bodily fluid sample obtained from the implant site. If the at least one biomarker is detected in the sample, the test subject is diagnosed with ALTR, whereupon therapeutic intervention is recommended that may or may not include surgical intervention.

In one embodiment, the invention provides a method for diagnosing ALTR in a test subject with an implant by analyzing the presence of bone growth at the implant site. The method of the invention comprises measuring the presence of at least one biomarker of bone growth, remodeling, repair or wound healing selected from the group consisting of Osteopontin and Platelet-derived growth factor subunit B in a bodily fluid sample obtained from the implant site. If the at least one biomarker is detected in the sample, the test subject is diagnosed with ALTR, whereupon therapeutic intervention is recommended that may or may not include surgical intervention.

In one embodiment, the invention provides a method for diagnosing ALTR in a test subject with an implant by analyzing a bodily fluid sample from the implant site for the presence of a local inflammatory response. The method of the invention comprises measuring the level of Pentraxin-3 in a bodily fluid sample from the implant site. If an increase in the level of Pentraxin-3 is detected in the bodily fluid sample obtained from the implant site as compared to a control level, and there are no indications of infection the test subject is diagnosed with ALTR, whereupon therapeutic intervention is recommended that may or may not include surgical intervention.

In one embodiment, the invention provides a method for diagnosing ALTR in a test subject with an implant by analyzing a bodily fluid sample from the implant site for the presence of a systemic inflammatory response. The method of the invention comprises measuring the level of C-reactive protein in a bodily fluid sample from the implant site. If a decrease or a normal level of C-reactive protein is detected in the bodily fluid sample obtained from the implant site as compared to a control level, and the subject has a combination of other markers indicating ALTR, the test subject is diagnosed with ALTR, whereupon therapeutic intervention is recommended that may or may not include surgical intervention. In some embodiments the ALTR exhibited by the test subject is related to at least one condition selected from the group consisting of hypersensitivity, metal hypersensitivity and tissue necrosis.

In one embodiment, the invention provides a method of diagnosing or predicting an ALTR in a test subject with an implant (e.g. a subject who has received a MOM joint replacement). The method comprises detecting the presence of at least one nucleic acid or protein biomarker in a bodily fluid sample obtained from a joint in the test subject. In some embodiments, the level of at least one nucleic acid or protein biomarker in a bodily fluid sample from the test subject is compared to a control level of the at least one nucleic acid or protein biomarker, wherein a difference in level of the at least one nucleic acid or protein biomarker in a bodily fluid sample in the test subject's sample as compared with the control level is indicative of an ALTR in the test subject. In other embodiments, the change in the level of at least one nucleic acid or protein biomarker between bodily fluid samples collected from the test subject at two or more different times during a preoperative visit and before a revision surgery is compared to a control level of the at least one nucleic acid or protein biomarker. In yet other embodiments, a difference in the level of the at least one nucleic acid or protein biomarker in the test subject's bodily fluid sample as compared to the control level is indicative of an ALTR, in the test subject. When such difference is detected surgical intervention is recommended for the test subject.

In one embodiment, the invention provides a method of monitoring the effectiveness of a treatment of an ALTR in a test subject with an implant. The method comprises detecting the presence of at least one nucleic acid or protein biomarker in a bodily fluid sample obtained from a joint in the test subject. In some embodiments, the method comprises comparing the level of at least one nucleic acid or protein biomarker in a bodily fluid sample from the test subject to a control level of the at least one nucleic acid or protein biomarker, wherein a difference in level of the at least one nucleic acid or protein biomarker in the test subject's bodily fluid sample as compared to the control level indicates that the treatment of the ALTR in the test subject is or is not effective.

In one embodiment, the invention provides a method of distinguishing between ALTR and PJI in a test subject with an implant. The method of the invention comprises requesting a test to determine whether the test subject has at least one biomarker in a bodily fluid sample obtained from the implant site of the test subject. The test may distinguish between the presence or absence of the biomarker in the test sample, or it may identify a level of a biomarker in the test sample that differs from a control level of a biomarker as that term is defined herein. The level of the at least one biomarker from the test subject is assessed and compared to a control level, wherein the presence, absence, increase or decrease in the level of the biomarker in the test subject's bodily fluid sample as compared with the control level can distinguish an ALTR and PJI in the test subject. When such presence, increase or decrease is detected, it can be indicative of an ALTR in the test subject and therapeutic intervention is recommended that may or may not include surgical intervention.

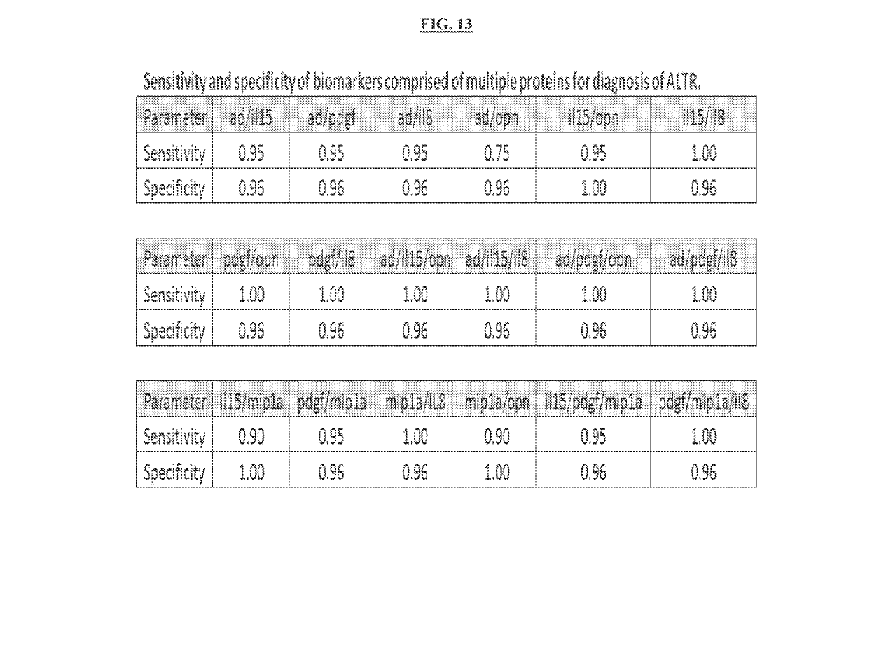

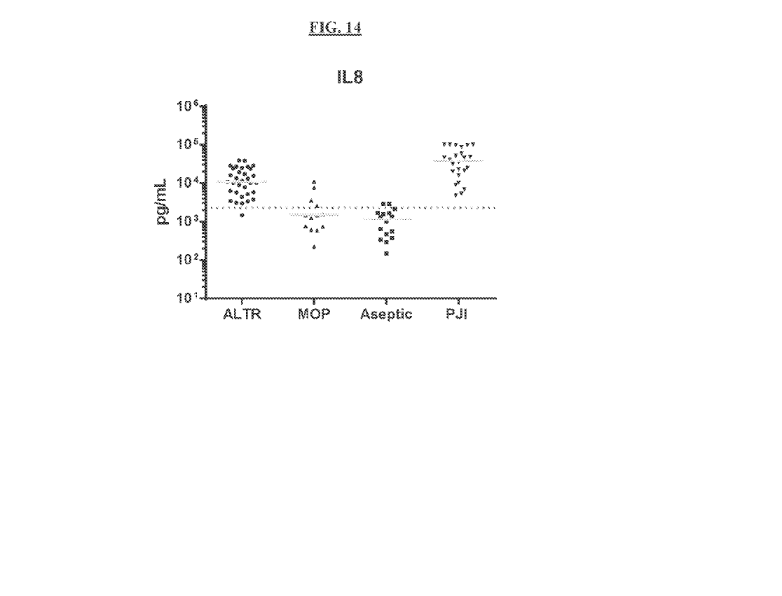

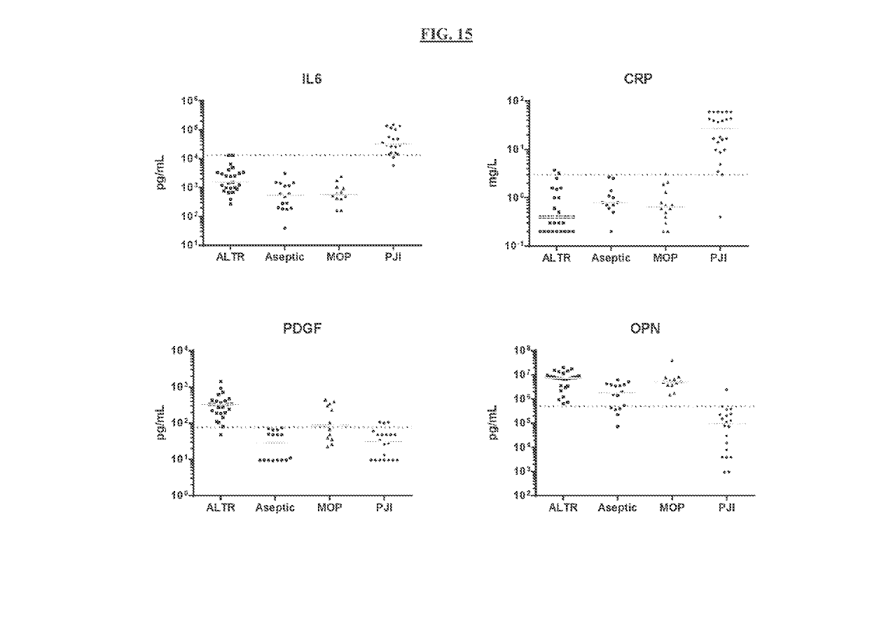

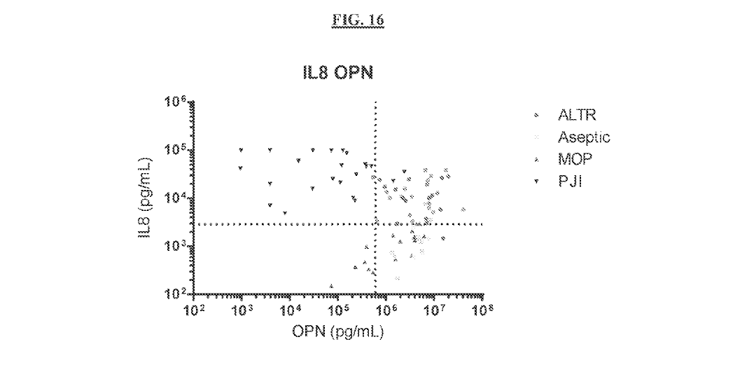

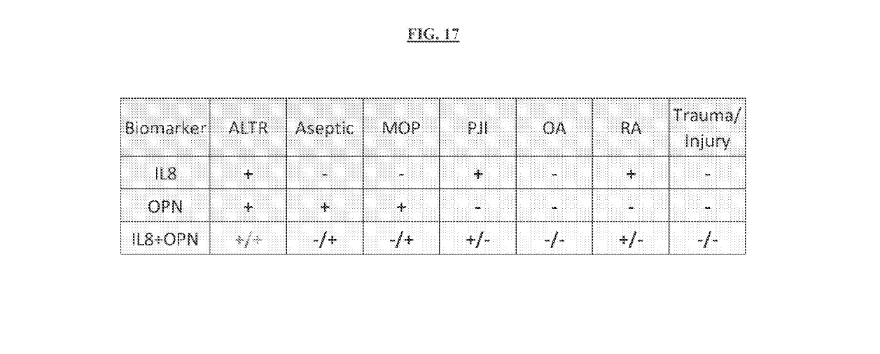

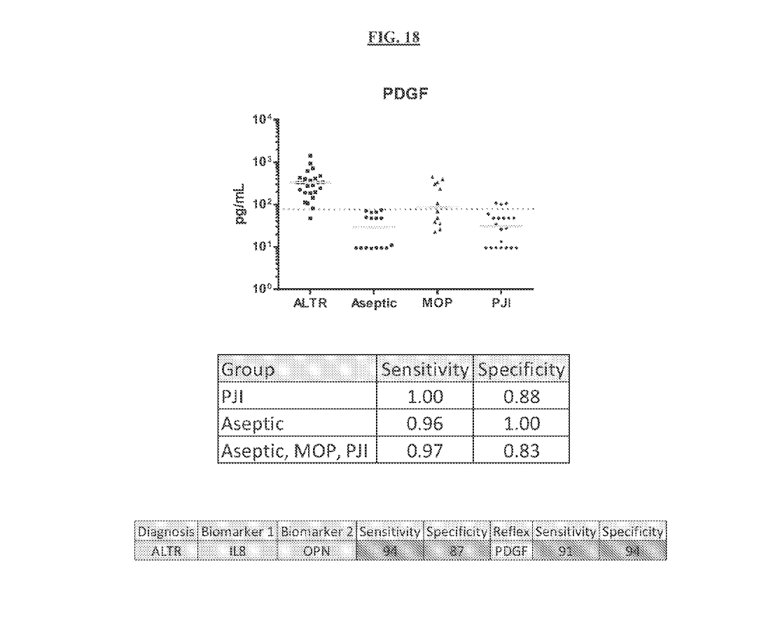

In one embodiment, the invention provides an algorithm for distinguishing between ALTR and PJI in a test subject having an implant. The algorithm of the invention comprises a first test to eliminate non-ALTR/non-PJI samples with a high negative predictive value, and a second test of the remaining samples to diagnose ALTR and PJI. When the distinction between ALTR and PJI is indicated, a therapeutic intervention, appropriate for the condition of the diagnosed test subject is recommended. In some aspects, the first test may comprise distinguishing between the presence or absence of or may identify a level in the test sample that differs from a control level for one or more biomarkers where the one or more biomarkers may comprise IL-8 and/or OPN. In some aspects the second test comprises assessing PDGF and in some aspects PDFG AB/BB. (See FIG. 14-18).

In other embodiments, the method comprises comparing the change in the level of at least one nucleic acid or protein biomarker between a test subject's bodily fluid sample collected at two or more different times during a preoperative visit and before a revision surgery to a control level of the at least one nucleic acid or protein biomarker.

In yet other embodiments, a difference in the level of the at least one nucleic acid or protein biomarker in test subject's bodily fluid sample as compared with the control level of the same nucleic acid or protein biomarker is indicative that the treatment of ALTR in the test subject is or is not effective.

In further embodiments, when the treatment of the ALTR is shown to be ineffective, the method of the invention includes recommending at least one selected from the group consisting of a modification in the treatment modalities, a change in the type of treatment and/or a surgical intervention for the test subject.

In some embodiments, the bodily fluid is from a joint. In other embodiments the bodily fluid is blood, serum, or synovial fluid.

### Processing of Synovial Fluid

Synovial fluid is a biological fluid that is found in the synovial cavity of the joints (e.g., knee, hip, shoulder, ankle, and wrist) of the human body between the cartilage and synovium of facing articulating surfaces. Synovial fluid provides nourishment to the cartilage and also serves as a lubricant for the joints. The cells of the cartilage and synovium (i.e. synoviocytes) secrete fluid and the fluid lubricates and reduces friction between the articulating surfaces.

Human synovial fluid is comprised of approximately 85% water. It is derived from the dialysate of blood plasma, which itself is made up of water, dissolved proteins, glucose, clotting factors, mineral ions, hormones, etc. The proteins, albumin and globulins, are present in synovial fluid and are believed to play an important role in the lubrication of the joint area. Other proteins are also found in human synovial fluid, including the glycoproteins such as alpha-1-acid glycoprotein (AGP), alpha-1-antitrypsin (A1AT) and lubricin.

Another compound that is present in human synovial fluid is hyaluronic acid. Hyaluronic acid is also believed to play a role in lubrication and is the primary component contributing to synovial fluid viscosity. Synovial fluid can be withdrawn from a desired joint for use in the diagnostic system of the invention. The synovial fluid withdrawn can be analyzed in order to ascertain the local condition in the joint.

Synovial fluid may be tested without any pretreatment; however, synovial fluid is inherently viscous and presents significant issues when the sample is aspirated or pipetted. Without wishing to be bound by any particular theory, the ideal diluent for synovial fluid enables extraction of the biomarker(s) and maintenance of the biomarker(s) in a state of detectability and contains a buffer capable of maintaining a pH in the range of 6-8. Preferably, the buffer (e.g. phosphate, Tris) contains saline as a base (i.e. NaCl). In one embodiment, the buffer contains a detergent that is capable of lysing the cells in the synovial fluid sample.

Detergents are amphipathic molecules, meaning they contain both a nonpolar “tail” having aliphatic or aromatic character and a polar “head.” Ionic character of the polar head group forms the basis for broad classification of detergents; they may be ionic (charged, either anionic or cationic), nonionic (uncharged) or zwitterionic (having both positively and negatively charged groups but with a net charge of zero). Detergent molecules allow the dispersion (miscibility) of water-insoluble, hydrophobic compounds into aqueous media, including the extraction and solubilization of membrane proteins.

In one embodiment, the buffer of the invention comprises one or more non-ionic detergents, including, but not limited to, n-octyl-p-D-glucopyranside, n-octyl-p-D-maltoside, ZWITTERGENT 3.14, deoxycholate; n-Dodecanoylsucrose; n-Dodecyl-p-D-glucopyranoside; n-Dodecyl-p-D-maltoside; n-Octyl-p-D-glucopyranoside; n-Octyl-p-D-maltopyranoside; n-Octyl-(3-D-thioglucopyranoside; n-Decanoylsucrose; n-Decy 1-p-D-maltopyranoside; n-Decyl-p-D-thiomaltoside; n-Heptyl-(3-D-glucopyranoside; n-Heptyl-(3-D-thioglucopyranoside; n-Hexyl-p-D-glucopyranoside; n-Nonyl-p-D-glucopyranoside; n-Octanoylsucrose; n-Octyl-p-D-5-glucopyranoside; n-Undecyl-p-D-maltoside; AP0-10; AP0-12; Big CHAP; Big CHAP, Deoxy; BRIJ® 35; C12E5; Ci2E₆; ĈEs; C12E9; Cyclohexyl-n-ethyl-p-D-maltoside; Cyclohexyl-n-hexyl-p-D-maltoside; Cyclohexyl-n-methyl-p-D-maltoside; Digitonin; ELUGENT™; GENAPOL® C-100; GENAPOL® X-080; GENAPOL® X-100; HECAMEG; MEGA-10; MEGA-8; MEGA-9; NOGA; NP-40; PLURONIC® 10 F-127; TRITON® X-100; TRITON® X-1 14; TWEEN® 20; or TWEEN® 80. Additionally, an ionic detergent can be used with the methods of the invention, including, but not limited to BATC, Cetyltrimethylammonium Bromide, Chenodeoxycholic Acid, Cholic Acid, Deoxycholic Acid, Glycocholic Acid, Glycodeoxycholic Acid, Glycolithocholic Acid, Lauroylsarcosine, 15 Taurochenodeoxycholic Acid, Taurocholic Acid, Taurodehydrocholic Acid, Taurolithocholic Acid, Tauroursodeoxycholic Acid, and TOPPA. Zwitterionic detergents can also be used with the compositions and methods of the invention, including, but not limited to, amidosulfobetaines, CHAPS, CHAPSO, carboxybetaines, and methylbetaines. Anionic detergents can also be used with the 20 compositions and methods of the invention, including, but not limited to, e.g. SDS, N-lauryl sarcosine, sodium deoxycholate, alkyl-aryl sulphonates, long chain (fatty) alcohol sulphates, olefine sulphates and sulphonates, alpha olefine sulphates and sulphonates, sulphated monoglycerides, sulphated ethers, sulphosuccinates, alkane sulphonates, phosphate esters, alkyl isethionates, and sucrose esters. Generally any suitable liquid (e.g. water) may be used as a solvent in the buffer of the present invention. The liquid may be organic or inorganic and may be a pure liquid, a mixture of liquids or a solution of substances in the liquid and may contain additional substances to enhance the properties of the solvent. Any liquid that is suitable for solubilizing the cellular components of body samples in total or in parts may be regarded as a lysis buffer as used herein.

In one embodiment, the solvent is designed, so that cells, cell debris, nucleic acids, polypeptides, lipids and other biomolecules potentially present in the sample are dissolved. In further embodiments of the present invention, the solvent may be designed to assure differential lysis of specific components of the body sample, leaving other components undissolved.

In some instances, the lysis buffer of the invention comprises one or more agents that prevent the degradation of components within the sample. Such components may for example comprise enzyme inhibitors such as proteinase inhibitors, RNAse inhibitors, DNAse inhibitors, nuclease (e.g. endonucleases and exonucleases) inhibitors, etc. Proteinase inhibitors may e.g. comprise inhibitors of serine proteinases, inhibitors of cysteine proteinases, inhibitors of aspartic proteinases, inhibitors of acidic proteinases, inhibitors of alkaline proteinases or inhibitors of neutral proteinases.

In one embodiment, the ideal diluent for processing synovial fluid contains a buffer capable of maintaining a pH in the range of about 5 to about 9, preferably about 6 to about 8, more preferably about 7 to about 8. Suitable, but non-limiting, buffers include HEPES, PIPES, Tris-Hydrochloride (Tris-HCl), and MOPS.

Optional components for the diluent may be included as part of the composition or as an adjuvant to be added separately, depending on what subsequent purification procedures are performed. Optional components include a defoaming agent at a concentration of about 1%; enzymes such as hyaluronidase lysozyme, lyticase, zymolyase, neuraminidase, streptolysin, cellulysin, mutanolysin, chitinase, glucalase or lysostaphin may be used, at a concentration of about 0.1 to 5 mg/ml; one or more inorganic salts such as sodium chloride, potassium chloride, magnesium chloride, calcium chloride, lithium chloride, or praseodymium chloride at a concentration of about 1 mM to 5M; protease inhibitors (e.g., phenylmethylsulfonyl fluoride, trypsin inhibitor, aprotinin, pepstatin A), reducing reagents (e.g., 2-mercaptoethanol and dithiothreitil) at concentrations of 0.1 to 10 mM; chelating agents (e.g., disodium ethylenediaminetetraacetic acid (Na2EDTA), EGTA, CDTA, most preferably at a concentration of about 1 mM or less); one or more ribonucleases (RNase A, T1, T2, and the like) at concentrations ranging from 1 to 400 ug/ml, or any combination of the 30 foregoing. DNase I concentrations may range from 1 to 100 units (10,000 units/mg). Preservatives such as Proclin 950 can be added to the diluent in order to preserve the solution comprising synovial fluid from degradation.

The diluent may also include the addition of heterophilic and Rf factor blocking agents to remove the impact of anti-species antibodies and Rf factor that may exist in the clinical sample. Reagents and methods of the present disclosure generally inhibit interferents from interfering with analysis for a particular analyte. Therefore, it is desirable to substantially suppress a false positive or a false negative signal caused by an interferent, if present, in a sample. In one aspect, such interferents may be, e.g., a heterophilic antibody, a rheumatoid factor, a lipoprotein, a fibrin, a clotting factor, an IgE, a human antibody to allergens, a human anti-mouse immunoglobulin, a human anti-goat immunoglobulin, a human anti-bovine immunoglobulin, a human anti-dog immunoglobulin and a human anti-rabbit immunoglobulin, etc.

Generally, interfering factors (interferents) such as heterophilic antibodies can arise from iatrogenic and noniatrogenic causes. The former may result from the normal response of the human immune system to an administered “foreign” protein antigen. The use of diagnostic or pharmaceutical reagents may lead to the introduction of such proteins and subsequent generation of such antibodies. For example, mouse monoclonal antibodies are foreign proteins in humans and in vivo they may trigger an immune response to produce human anti-mouse antibodies. In many circumstances where mouse monoclonal antibodies have been administered to subjects, those subjects have developed a human anti-mouse antibody response.

Accordingly, it is desirable to process synovial fluid and to arrive at an assay buffer that: 1) dilutes the synovial fluid sample to enhance the ability to pipette/transfer the sample, 2) optionally lyses all of the cellular components in the synovial fluid sample, 3) preserves the synovial fluid sample and stabilizes the biomarkers therefrom, and 4) renders inert/complexes/removes interfering substances from the synovial fluid sample.

In some instances, it is desirable to centrifuge (e.g., spin) the synovial fluid sample prior to assaying the sample. For example, if there is some contamination of the synovial fluid with blood or if the sample contains particulate debris, it is desirable to spin the sample prior to processing in the assay.

### Identifying a Marker or Biomarker

The invention includes methods for the identification of differentially expressed nucleic acids or protein biomarkers in a bodily fluid sample from a joint that indicate the test subject (i.e. patient) is experiencing an ALTR (e.g. including metal hypersensitivity) as a result of an implant (e.g. MOM joint replacement). The method of identification of such biomarkers includes use of control samples from for instance, either a subject with OA but without any joint surgery and/or from an asymptomatic subject with joint replacement surgery (aseptic), and/or from a periprosthetic joint infection (PJI) subject with joint replacement surgery.

In one embodiment, the joint can be a native joint (e.g., OA, RA, Gout, and PseudoGout) or a replacement joint.

The invention contemplates the identification of differentially expressed biomarkers by multianalyte assay profiling (MAP) or by whole genome nucleic acid microarray, to identify biomarkers differentially expressed between non-ALTR joints and ALTR joints. The invention further contemplates using methods known to those skilled in the art to detect and to measure the level of differentially expressed biomarker or biomarker expression products, such as RNA and protein, to measure the level of one or more differentially expressed biomarker or biomarker expression products.

In one embodiment, the invention includes a gene signature differential analysis method designed to detect genes present in one sample set, and absent in another. Genes with differential expression between the tested samples and the control samples are better diagnostic and therapeutic targets than genes that do not change in expression.

Analysis for the purpose of monitoring differential gene expression may be focused on a variety of tissues and fluids, and may also be used to detect or measure a number of different molecular targets. When a cell expresses a gene, it transcribes the appropriate RNA, which is ultimately translated into a protein. The relevant protein may then be localized to a variety of intracellular or extracellular locations.

Methods of detecting or measuring protein concentration or gene expression may utilize methods that focus on cellular components (cellular examination), or methods that focus on examining extracellular components (fluid examination). Because gene expression involves the ordered production of a number of different molecules, a cellular or fluid examination may be used to detect or measure a variety of molecules including RNA, protein, and a number of molecules that may be modified as a result of the protein's function.

The practice of the present invention may also employ software and systems. Computer software products of the invention typically include computer readable media having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170. Additionally, the present invention may have preferred embodiments that include methods for providing genetic information over networks such as the Internet as shown in US Pub No 20020183936.

The genes identified as being differentially expressed may be assessed in a variety of nucleic acid detection assays to detect or quantify the expression level of a gene or multiple genes in a given sample. For example, traditional Northern blotting, nuclease protection, RT-PCR, microarray, and differential display methods may be used for detecting gene expression levels. Methods for assaying for mRNA include Northern blots, slot blots, dot blots, and hybridization to an ordered array of oligonucleotides. Any method for specifically and quantitatively measuring a specific protein or mRNA or DNA product can be used. However, methods and assays are most efficiently designed with array or chip hybridization-based methods for detecting the expression of a large number of genes. Any hybridization assay format may be used including, but not limited to, solution-based and solid support-based assay formats.

The protein products of the genes identified herein can also be assayed to determine the amount of expression. Methods for assaying for a protein include but are not limited to Western blot, immunoprecipitation, immunoassay, immunohistochemistry, immunofluorescence and radioimmunoassay. The proteins analyzed may be localized intracellularly (most commonly an application of immunohistochemistry) or extracellularly.

The identification of biomarkers of the present invention may be accomplished using various suitable assays. A suitable assay may include one or more of a chemical assay, an enzyme assay, an immunoassay, mass spectrometry, chromatography, electrophoresis, a biosensor, an antibody microarray or any combination thereof. Most commonly if an immunoassay is used it may be an enzyme-linked immunosorbant assay (ELISA), a sandwich assay, a competitive or a non-competitive assay, a radioimmunoassay (RIA), a lateral flow immunoassay, a Western Blot, an electro-chemilumescent assay, a magnetic particle assay, an immunoassay using a biosensor, a bead-based array assay (e.g. Luminex, Milliplex or Bioplex) an immunoprecipitation assay, an agglutination assay, a turbidity assay or a nephelometric assay. A detailed description on immunoassays, mass spectrometry and chromatography known in the art is provided elsewhere herein.

The invention described herein also relates to methods for a multiplex analysis platform. In one embodiment, the method comprises an analytical method for multiplexing analytical measurements of markers. In another embodiment, the method comprises a set of compatible analytical strategies for multiplex measurements of markers in bodily fluid samples (e.g. synovial fluid, whole blood, plasma or serum).

### Immunoassays

In one embodiment, the methods of the invention can be performed in the form of various immunoassay formats, which are well known in the art. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), enzyme linked immunospot assay (ELISPOT), radioimmunoassays (RIA), radioimmune precipitation assays (RIPA), immunobead capture assays, Western blotting, dot blotting, gel-shift assays, Flow cytometry, protein arrays, antigen arrays, antibody arrays, multiplexed bead arrays, magnetic capture, in vivo imaging, fluorescence resonance energy transfer (FRET), fluorescence recovery/localization after photobleaching (FRAP/FLAP), a sandwich assay, a competitive assay, an immunoassay using a biosensor, an immunoprecipitation assay, an agglutination assay, a turbidity assay, a nephelometric assay, immunoPCR, Quanterix, Singulex, AlphaLISA, Siscapa, Luminex, Singulex Brenna® immunoassay, TR-FRET, Meso-scale discovery (MSD), lateral flow immunochromatographic device, automated magnetic particle assay, fluorescent polarization, chemiluminescence, electrochemiluminescence, etc.

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species or unbound proteins, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or tag, such as any radioactive, colored, chemiluminescent, fluorescent, biological or enzymatic tags or any other known label. See, for example, U.S. Pat. Nos. 3,817,837; 3,850,752; 3,939,350; 3,996,345; 4,277,437; 4,275,149 and 4,366,241, each of which is incorporated herein by reference in its entirety and specifically for teachings regarding immunodetection methods and labels.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody. Fluorophores are compounds or molecules that fluoresce. Fluorophores absorb electromagnetic energy at one wavelength and emit electromagnetic energy at a second wavelength.

There are two main types of immunoassays, homogeneous and heterogeneous. In homogeneous immunoassays, both the immunological reaction between an antigen and an antibody and the detection are carried out simultaneously in a homogeneous reaction. Heterogeneous immunoassays include at least one separation step between bound and unbound label, which allows the differentiation of reaction products from unreacted reagents. A variety of immunoassays can be used to detect one or more of the proteins disclosed or incorporated by reference herein.

ELISA and Luminex bead based array platforms are examples of heterogeneous immunoassays, which can be used in the methods disclosed herein. These assays can be used to detect protein antigens in various formats. In the “sandwich” format the antigen being assayed is held between two antibodies. In this method, a solid surface is first coated with a solid phase antibody. The test sample, containing the antigen (e.g., a diagnostic protein), or a composition containing the antigen, such as a synovial fluid sample from a subject of interest, is then added and the antigen is allowed to react with the bound antibody. Any unbound antigen is washed away. A known amount of enzyme-labeled antibody is then allowed to react with the bound antigen. Any excess unbound enzyme-linked antibody is washed away after the reaction. The substrate specific for the enzyme used in the assay is then added and the reaction between the substrate and the enzyme produces a color change. The amount of visual color change is a direct measurement of specific enzyme-conjugated bound antibody, and consequently directly proportional to the amount of antigen present in the sample tested.

ELISA can also be used as a competitive assay. In the competitive assay format, the test specimen containing the antigen to be determined is mixed with a precise amount of enzyme-labeled antigen and both compete for binding to an anti-antigen antibody attached to a solid surface. Excess free enzyme-labeled antigen is washed off before the substrate for the enzyme is added. Alternatively the antigen can be coated onto the solid surface which competes with an antigen in the sample for labeled antigen-specific antibody. The amount of color intensity resulting from the enzyme-substrate interaction is inversely proportional to the amount of antigen in the sample tested. A heterogeneous immunoassay, such as an ELISA, can be used to detect any of the proteins disclosed or incorporated by reference herein.

Homogeneous immunoassays include, for example, alphaLISA, TRFRET (time-resolved fluorescence energy transfer), and the Enzyme Multiplied Immunoassay Technique (EMIT), which typically includes a biological sample comprising the biomarkers to be measured, enzyme-labeled molecules of the biomarkers to be measured, specific antibody or antibodies binding the biomarkers to be measured, and a specific enzyme chromogenic substrate. In a typical EMIT, excess of specific antibodies is added to a biological sample. If the biological sample contains the proteins to be detected, such proteins bind to the antibodies. A measured amount of the corresponding enzyme-labeled proteins is then added to the mixture. Antibody binding sites not occupied by molecules of the protein in the sample are occupied with molecules of the added enzyme-labeled protein. As a result, enzyme activity is reduced because only free enzyme-labeled protein can act on the substrate. The amount of substrate converted from a colorless to a colored form determines the amount of free enzyme left in the mixture. A high concentration of the protein to be detected in the sample causes higher absorbance readings. Less protein in the sample results in less enzyme activity and consequently lower absorbance readings. Inactivation of the enzyme label when the antigen-enzyme complex is antibody-bound makes the EMIT a useful system, enabling the test to be performed without a separation of bound from unbound compounds as is necessary with other immunoassay methods. A homogenous immunoassay, such as an EMIT, can be used to detect any of the proteins disclosed or incorporated by reference herein.

In many immunoassays, as described elsewhere herein, detection of antigen is made with the use of antigen specific antibodies as detector molecules. However, immunoassays and the systems and methods of the present invention are not limited to the use of antibodies as detector molecules. Any substance that can bind or capture the antigen within a given sample may be used. Aside from antibodies, suitable substances that can also be used as detector molecules include but are not limited to enzymes, peptides, proteins, receptors, and nucleic acids. Further, there are many detection methods known in the art in which the captured antigen may be detected. In some assays, enzyme-linked antibodies produce a color change. In other assays, detection of the captured antigen is made through detecting fluorescent, luminescent, chemiluminescent, or radioactive signals. The system and methods of the current invention is not limited to the particular types of detectable signals produced in an immunoassay.

Immunoassay kits are also included in the invention. These kits include, in separate containers (a) polyclonal and/or monoclonal antibodies having binding specificity for the biomarkers (e.g. polypeptides) used in the diagnosis of ALTR; and (b) and anti-antibody immunoglobulins. This immunoassay kit may be utilized for the practice of the various methods provided herein. The primary antibody in (a) can be directly labeled and in this case there is no need for an anti-antibody immunoglobulin (b). The monoclonal antibodies and the anti-antibody immunoglobulins can be provided in an amount of about 0.001 g to 100 ug, and more preferably about 0.01 ug to 10 ug. The antibody detection reagent may be a polyclonal immunoglobulin, protein A or protein G or functional fragments thereof, which may be labeled prior to use by methods known in the art. In several embodiments, the immunoassay kit includes two, three or four of: antibodies that specifically bind a biomarker protein(s) disclosed or incorporated herein.

In one embodiment, the lateral flow immunoassay kit of the invention can comprise (a) a sample pad, (b) a conjugated label pad, the conjugated label pad having a detectable label, a portion of the conjugated label pad and a portion of the sample pad forming a first interface, (c) a lateral-flow assay comprising a membrane, a portion of the membrane and a portion of the conjugated label pad forming a second interface, and (d) at least one antibody bound to the membrane, the first interface allowing fluid to flow from the sample pad to the conjugated label pad and contact the detectable label wherein the biomarker present in the sample forms a biomarker-conjugated label complex, the second interface allowing fluid to flow from the conjugated label pad to the membrane and to contact the at least one membrane-bound antibody to form a biomarker-antibody complex and cause the detectable label to form a detectable signal. Other reagent format configurations known to one skilled in the art are included herein.

### Mass Spectrometry and Chromatography

In one embodiment, the methods of the invention can be performed in the form of various mass spectrometry (MS) or chromatography formats, which are well known in the art. As such, the levels of biomarkers present in a sample can be determined by mass spectrometry. Generally, any mass spectrometric techniques that can obtain precise information on the mass of peptides, and preferably also on fragmentation and/or (partial) amino acid sequence of selected peptides, are useful herein. Suitable peptide MS techniques and systems are well-known per se (see, e.g., Methods in Molecular Biology, vol. 146: “Mass Spectrometry of Proteins and Peptides”, by Chapman, ed., Humana Press 2000, ISBN 089603609x; Biemann 1990. Methods Enzymol 193: 455-79; or Methods in Enzymology, vol. 402: “Biological Mass Spectrometry”, by Burlingame, ed., Academic Press 2005, ISBN 10 9780121828073) and may be used herein.

The terms “mass spectrometry” or “MS” as used herein refer to methods of filtering, detecting, and measuring ions based on their mass-to-charge ratio, or “m/z.” In general, one or more molecules of interest are ionized, and the ions are subsequently introduced into a mass spectrographic instrument where, due to a combination of magnetic and electric fields, the ions follow a path in space that is dependent upon mass (“m”) and charge (“z”). For examples see U.S. Pat. Nos. 6,204,500, 6,107,623, 6,268,144, 6,124,137; Wright et al., 1999, Prostate Cancer and Prostatic Diseases 2: 264-76; Merchant et al., 2000, Electrophoresis 21: 1164-67, each of which is hereby incorporated by reference in its entirety, including all tables, figures, and claims. Mass spectrometry methods are well known in the art and have been used to quantify and/or identify biomolecules, such as proteins and hormones (Li et al., 2000, Tibtech. 18:151-160; Starcevic et. al., 2003, J. Chromatography B, 792: 197-204; Kushnir et. al., 2006, Clin. Chem 52:120-128; Rowley et al., 2000, Methods 20: 383-397; Kuster et al., 1998, Curr. Opin. Structural Biol. 8: 393-400). Further, mass spectrometric techniques have been developed that permit at least partial de novo sequencing of isolated proteins (Chait et al., 1993, Science, 262:89-92; Keough et al., 1999, Proc. Natl. Acad. Sci. USA 96:7131-6; Bergman, 2000, EXS 88:133-44). Various methods of ionization are known in the art. For examples, Atmospheric Pressure Chemical Ionization (APCI) Chemical Ionization (CI) Electron Impact (EI) Electrospray Ionization (ESI), Fast Atom Bombardment (FAB), Field Desorption/Field Ionization (FD/FI), Matrix Assisted Laser Desorption Ionization (MALDI), and Thermospray Ionization (TSP).

The levels of biomarkers present in a sample can be determined by MS such as matrix-assisted laser desorption/ionization time-of-flight (MALDI-TOF) MS; MALDI-TOF post-source-decay (PSD); MALDI-TOF/TOF; surface-enhanced laser desorption/ionization time-of-flight mass spectrometry (SELDI-TOF) MS; tandem mass spectrometry (e.g., MS/MS, MS/MS/MS etc.); electrospray ionization mass spectrometry (ESI-MS); ESI-MS/MS; ESIMS/(MS)n (n is an integer greater than zero); ESI 3D or linear (2D) ion trap MS; ESI triple quadrupole MS; ESI quadrupole orthogonal TOF (Q-TOF); ESI Fourier transform MS systems; desorption/ionization on silicon (DIOS); secondary ion mass spectrometry (SIMS); atmospheric pressure chemical ionization mass spectrometry (APCI-MS); APCI-MS/MS; APCI-(MS)”; atmospheric pressure photoionization mass spectrometry (APPI-MS); APPIMS/MS; APPI-(MS)”; liquid chromatography-mass spectrometry (LC-MS), gas chromatography-mass spectrometry (GC-MS); high performance liquid chromatography-mass spectrometry (HPLC-MS); capillary electrophoresis-mass spectrometry; and nuclear magnetic resonance spectrometry. Peptide ion fragmentation in tandem MS (MS/MS) arrangements may be achieved using manners established in the art, such as, e.g., collision induced dissociation (CID). See for example, U.S. Patent Application Nos: 20030199001, 20030134304, 20030077616, which are herein incorporated by reference in their entirety. Such techniques may be used for relative and absolute quantification and also to assess the ratio of the biomarker according to the invention with other biomarkers that may be present. These methods are also suitable for clinical screening, prognosis, monitoring the results of therapy, identifying patients most likely to respond to a particular therapeutic treatment, for drug screening and development, and identification of new targets for drug treatment.

In certain embodiments, a gas phase ion spectrophotometer is used. In other embodiments, laser-desorption/ionization mass spectrometry is used to analyze the sample. Modem laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them See, e.g., U.S. Pat. No. 5,118,937, and U.S. Pat. No. 5,045,694. In SELDI, the substrate surface is modified so that it is an active participant in the desorption process. In one variant, the surface is derivatized with adsorbent and/or capture reagents that selectively bind the biomarker of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. SELDI is a powerful tool for identifying a characteristic “fingerprint” of proteins and peptides in body fluids and tissues for a given condition, e.g. drug treatments and diseases. This technology utilizes protein chips to capture proteins/peptides and a time-of-flight mass spectrometer (tof-MS) to quantitate and calculate the mass of compounds ranging from small molecules and peptides of less than 1,000 Da up to proteins of 500 kDa. Quantifiable differences in protein/peptide patterns can be statistically evaluated using automated computer programs which represent each protein/peptide measured in the biofluid spectrum as a coordinate in multi-dimensional space. The SELDI 15 system also has a capability of running hundreds of samples in a single experiment. In addition, all the signals from SELDI mass spectrometry are derived from native proteins/peptides (unlike some other proteomics technologies which require protease digestion), thus directly reflecting the underlying physiology of a given condition.

In MALDI and SELDI, the derivatizing agent generally is localized to a specific location on the substrate surface where the sample is applied. See, e.g., U.S. Pat. No. 5,719,060 and WO 98/59361. The two methods can be combined by, for example, using a SELDI affinity surface to capture an analyte and adding matrix-containing liquid to the captured analyte to provide the energy absorbing material. For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4th ed. Vol. 15 (John Wiley & Sons, New York 1995), pp. 1071-1094. Detection and quantification of the biomarker will typically depend on the detection of signal intensity. For example, in certain embodiments, the signal strength of peak values from spectra of a first sample and a second sample can be compared (e.g., visually, by computer analysis etc.), to determine the relative amounts of particular biomarker. Software programs such as the Biomarker Wizard program (Ciphergen Biosystems, Inc., Fremont, Calif.) can be used to aid in analyzing mass spectra. The mass spectrometers and their techniques are well known to those of skill in the art.

In an embodiment, detection and quantification of biomarkers by mass spectrometry may involve multiple reaction monitoring (MRM), such as described among others by Kuhn et al. 2004 (Proteomics 4: 1175-86).

In an embodiment, MS peptide analysis methods may be advantageously combined with upstream peptide or protein separation or fractionation methods, such as for example with the chromatographic and other methods described herein below.

Chromatography can also be used for measuring biomarkers. As used herein, the term “chromatography” encompasses methods for separating chemical substances, referred to as such and vastly available in the art. In a preferred approach, chromatography refers to a process in which a mixture of chemical substances (analytes) carried by a moving stream of liquid or gas (“mobile phase”) is separated into components as a result of differential distribution of the analytes, as they flow around or over a stationary liquid or solid phase (“stationary phase”), between said mobile phase and said stationary phase. The stationary phase may be usually a finely divided solid, a sheet of filter material, or a thin film of a liquid on the surface of a solid, or the like. Chromatography is also widely applicable for the separation of chemical compounds of biological origin, such as, e.g., amino acids, proteins, fragments of proteins or peptides, etc.

Chromatography as used herein may be preferably columnar (i.e., wherein the stationary phase is deposited or packed in a column), preferably liquid chromatography, and yet more preferably high-performance liquid chromatography (HPLC). While particulars of chromatography are well known in the art, for further guidance see, e.g., Meyer M., 1998, ISBN: 047198373X, and “Practical HPLC Methodology and Applications”, Bidlingmeyer, B. A, John Wiley & Sons Inc., 1993.

Exemplary types of chromatography include, without limitation, HPLC, normal phase HPLC (NP-HPLC), reversed phase HPLC (RP-HPLC), ion exchange chromatography (IEC), such as cation or anion exchange chromatography, hydrophilic interaction chromatography (HILIC), hydrophobic interaction chromatography (HIC), size exclusion chromatography (SEC) including gel filtration chromatography or gel permeation chromatography, chromatofocusing, affinity chromatography such as immuno-affinity, immobilized metal affinity chromatography, and the like.

In an embodiment, chromatography, including single-, two- or more-dimensional chromatography, may be used as a peptide fractionation or purification method in conjunction with a further peptide analysis method, such as for example, with a downstream mass spectrometry analysis as described elsewhere in this specification (e.g. stable isotope standard capture with anti-peptide antibodies (SISCAPA)).

Further peptide or polypeptide separation, identification or quantification methods may be used, optionally in conjunction with any of the above described analysis methods, for measuring biomarkers in the present disclosure. Such methods include, without limitation, chemical extraction partitioning, isoelectric focusing (IEF) including capillary isoelectric focusing (CIEF), capillary isotachophoresis (CITP), capillary electrochromatography (CEC), and the like, one-dimensional polyacrylamide gel electrophoresis (PAGE), two-dimensional polyacrylamide gel electrophoresis (2D-PAGE), two-dimensional difference in gel electrophoresis (2D-DIGE), capillary gel electrophoresis (CGE), capillary zone electrophoresis (CZE), micellar electrokinetic chromatography (MEKC), free flow electrophoresis (FFE), etc.

### Biosensors

In one embodiment, the biomarkers of the invention are detected using biosensors, e.g. with sensor systems with amperometric, electrochemical, potentiometric, conductimetric, impedance, magnetic, optical, acoustic or thermal transducers.

Generally, biosensors include a biosensor recognition element which can include proteins, nucleic acids, antibodies, etc. that bind to a particular biomarker and a transducer which converts a molecular signal (i.e. binding of biomarker to recognition element) into an electric or digital signal that can be quantified, displayed, and analyzed. Biosensors may also include a reader device which translates the signal into a user-friendly display of the results. Examples of potential components that comprise an exemplary biosensor are described in Bohunicky et al. (2011, Nanotechnology Science and Applications, 4: 1-10), which is hereby incorporated by reference in its entirety.

A biosensor may incorporate a physical, chemical or biological detection system. In one embodiment, a biosensor is a sensor with a biological recognition system, e.g. based on a nucleic acid, such as an oligonucleotide probe or aptamer, or a protein such as an enzyme, binding protein, receptor protein, transporter protein or antibody. In one embodiment, the biological recognition system may comprise traditional immunoassays described elsewhere herein. In another element, the recognition element (e.g. protein, nucleic acid, antibody, etc.) may be unlabeled and binding of the biomarker to the element is directly observed and converted into a signal by the transducer. A biosensor may include microfluidic means for measuring or dispensing volumes, housing reagents causing mixing, providing incubation by capillary flow, gravity, electro-motive force or other means to move fluid.

The method for detection of the biomarker in a biosensor uses immunological, electrical, thermal, magnetic, optical (e.g. hologram) or acoustic technologies. Using such biosensors, it is possible to detect the target biomarker at the anticipated concentrations found in biological samples.

The biosensor may incorporate detection methods and systems as described herein for detection of the biomarker. Biosensors may employ electrical (e.g. amperometric, potentiometric, conductimetric, or impedance detection systems), calorimetric (e.g. thermal), magnetic, optical (e.g. hologram, luminescence, fluorescence, colorimetry), or mass change (e.g. piezoelectric, acoustic wave) technologies. In a biosensor according to the invention the level of one, two, three, or more biomarkers can be detected by one or more methods selected from: direct, indirect or coupled enzymatic, spectrophotometric, fluorimetric, luminometric, spectrometric, polarimetric and chromatographic techniques. Particularly preferred biosensors comprise one or more enzymes used directly or indirectly via a mediator, or using a binding, receptor or transporter protein, coupled to an electrical, optical, acoustic, magnetic or thermal transducer. Using such biosensors, it is possible to detect the level of target biomarkers at the anticipated concentrations found in biological samples.

In one embodiment of a biosensor, a biomarker of the invention can be detected using a biosensor incorporating technologies based on “smart” holograms, or high frequency acoustic systems, such systems are particularly amenable to “bar code” or array configurations. In smart hologram sensors (Smart Holograms Ltd, Cambridge, UK), a holographic image is stored in a thin polymer film that is sensitized to react specifically with the biomarker. On exposure, the biomarker reacts with the polymer leading to an alteration in the image displayed by the hologram. The test result read-out can be a change in the optical brightness, image, color and/or position of the image. For qualitative and semi-quantitative applications, a sensor hologram can be read by eye, thus removing the need for detection equipment. A simple color sensor can be used to read the signal when quantitative measurements are required. Opacity or color of the sample does not interfere with operation of the sensor. The format of the sensor allows multiplexing for simultaneous detection of several substances. Reversible and irreversible sensors can be designed to meet different requirements, and continuous monitoring of a particular biomarker of interest is feasible.

Biosensors to detect the biomarker of the invention may include acoustic, surface plasmon resonance, holographic and microengineered sensors. Imprinted recognition elements, thin film transistor technology, magnetic acoustic resonator devices and other novel acousto-electrical systems may be employed in biosensors for detection of the biomarkers of the invention.

Suitably, biosensors for detection of the biomarker of the invention are coupled, i.e. they combine biomolecular recognition with appropriate means to convert detection of the presence, or quantitation, of the biomarker in the sample into a signal. Biosensors can be adapted for “alternate site” diagnostic testing, e.g. in the ward, outpatients' department, surgery, home, field and workplace.

### Control

In some instances, a control can be standardized and is used only for the purpose of establishing initial cutoffs for the assays of the invention. Therefore, in some instances, the methods of the invention can diagnose an ALTR, e.g., a metal hypersensitivity, without the need for comparison with a control. In other words, mere detection of a biomarker of the invention without the requirement of comparison to a control group can be used to diagnose an ALTR. In this manner, the system according to the present invention yields a qualitative (yes/no answer); semi-quantitative (−/+/++/+++/++++) or quantitative answer.

### Biomarkers

In one embodiment, the system disclosed herein includes application of bodily fluid (e.g., synovial fluid) obtained from a test subject to a system for the detection of one or more biomarkers that are differentially expressed (i.e. upregulated or downregulated) in a sample from a test joint. Such biomarkers include, but are not limited to, Neutrophil defensin 1 (Gene: DEF Al, Protein: Alpha Defensin), C-reactive protein (Gene: CRP, Protein: CRP), Growth-regulated alpha protein (Gene: CXCLI, protein: GRO), Neutrophil elastase (Gene: ELANE, Protein: HNE), Interferon gamma (Gene: IFNγ Protein: IFNG), Interleukin 1-alpha (Gene: IL-1A, Protein: IL-1α), Interleukin 1-beta (Gene: IL-1B, Protein: IL-1β), Interleukin 6 (Gene/protein: IL-6), Interleukin 8 (Gene: CXCL8, Protein: IL-8), Interleukin 12-beta (Gene: IL-12Beta, Protein: IL-12β), Interleukin 15 (Gene/Protein: IL-15), C-X-C motif chemokine 10 (Gene: CXCL10, Protein: IP-10), Lactate, Leptin (Gene: LEP, Protein: Leptin), Monocyte chemotactic protein 1 (Gene: CCL2, Protein: MCP-1), Monocyte chemotactic protein 3 (Gene: CCL7, Protein: MCP-3), C-C motifchemokine 22 (Gene: CCL22, Protein: MDC), Macrophage inflammatory protein 1-alpha (Gene: CCL3, Protein: MIP-1α), Tumor necrosis factor receptor superfamily member 11B (Gene: TNFRSIIB, Protein: OPG), Osteopontin (Gene: SPP1, Protein: OPN); Platelet-derived growth factor subunit B (Gene: PDGFB, Protein: PDGF-AB/BB), Tumor necrosis factor alpha (Gene: TNF-a, Protein: TNF-α), VEGF (Vascular endothelial growth factor), Pentraxin-3 (Gene: PTX3, Protein: PTX3), Tumor necrosis factor ligand superfamily member 6 (Gene: FASL, Protein: FASL), Soluble intercellular adhesion molecule-1 (Gene: sICAM-1; Protein:SICAM-1) and the like.

In one embodiment, the system disclosed herein includes application of a synovial fluid from a test sample to a system for the detection of one or more biomarkers that is upregulated or downregulated in a joint. In particular, the joint exhibits symptoms of an ALTR occurring in a subject who has received a MOM or MOP joint replacement.

The present invention is partly based on the discovery that the cells in an inflamed joint are different based on the nature of the disease and diagnostic gene/protein expression profiles in an ALTR is unique in a subject who has received a MOM joint replacement. As described elsewhere herein, comparison of the expression patterns of the sample to be tested with those of the controls is used to establish initial cutoffs for the biomarker levels of the invention.

The system of the invention can be used to detect at least one, two, three, four, five, at least ten different biomarkers, or a multitude of biomarkers. In some examples, the system includes determining a proteomic profile. In other examples, the system of the invention includes detecting a proteomic profile including at least 1, 2, 3, 4, 5, 6, 7, 8, 9, 10, or all of these proteins, including any of the proteins set forth in herein. In one embodiment of the invention, the system can detect nucleic acids that encode the protein biomarker or biomarkers of the invention.

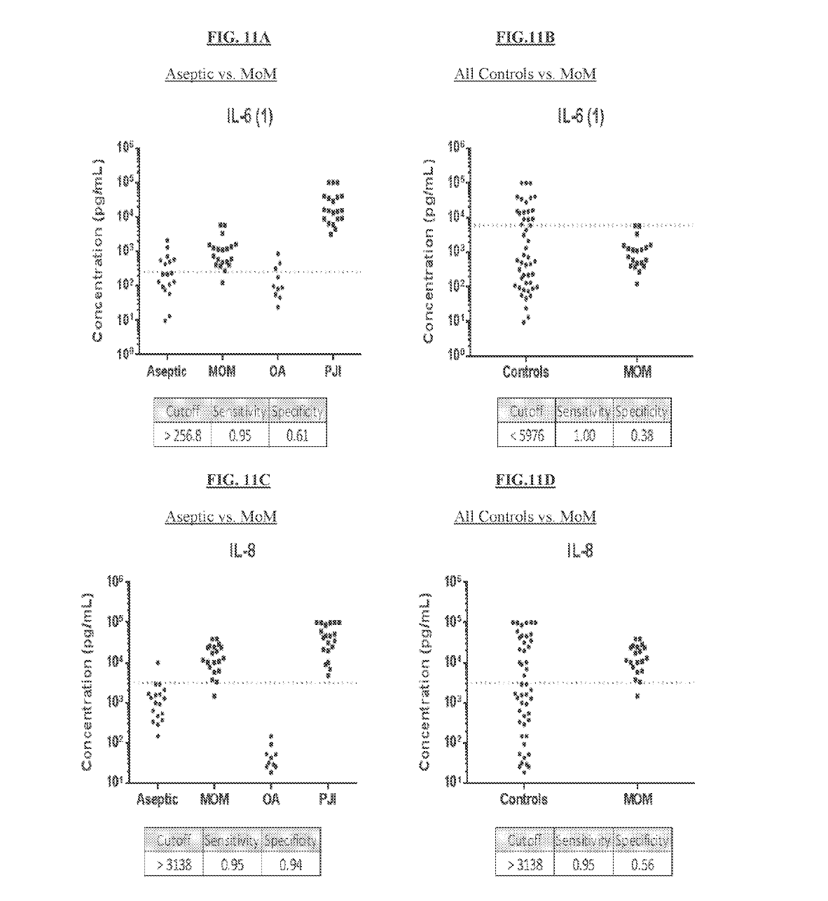

In one embodiment, the invention provides a system for detecting a biomarker of ALTR with at least 90%, 91%, 92%, 93%, 94%, 95%, 96%, 97%, 98%, 99%, 100% sensitivity; at least 90%, 91%, 92%, 93%, 94%, 95%, 96%, 97%, 98%, 99%, 100% specificity; or both at least 90%, 91%, 92%, 93%, 94%, 95%, 96%, 97%, 98%, 99%, 100% sensitivity and specificity. In one embodiment, the invention provides a system for detecting a biomarker of ALTR with at least 45%, 50%, 55%, 60%, 65%, 70%, 75%, 80%, 85%, 90% sensitivity and >99% specificity. In one embodiment, the invention provides a system for detecting a biomarker of inflammation in a joint with at least 90%, 91%, 92%, 93%, 94%, 95%, 96%, 97%, 98%, 99%, 100% accuracy. Examples of the cutoff value (derived by ROC analysis) for certain biomarkers are provided herein in FIGS. 8 and 12.

### Disease Condition

ALTR is one of the most common complications associated with MOM replacement hips. The condition can require extensive treatment, including total hip revision, or the surgical replacement of the all-metal hip for a different model. Metal particles released into the synovium can cause adverse reactions for body tissue including bone. The condition is harmful and degenerative, causing bone loss and fractures in the surrounding healthy bone. Inflammation, fluid accumulation, and tumor-like masses (pseudotumors) can develop in the body's soft tissue as well.

In one embodiment of the invention, detection of a marker in a sample identifies a subject from which the sample was obtained, as having or not having ALTR. For example, the invention provides the ability to detect a biomarker in a bodily fluid sample from a joint (synovial fluid sample), wherein detection of the biomarker identifies ALTR sufficient to warrant surgical intervention.

In one embodiment, the invention provides a system for quickly diagnosing whether the ALTR is present. Determination of ALTR informs the physician that surgical revision is recommended for the patient.

In one embodiment of the invention, detection of a marker in a sample identifies a subject from which the sample was obtained, as having ALTR and not PJI.

In one embodiment of the invention, detection of a marker in a sample differentiates a well functioning MOM joint from an ALTR.

In one embodiment of the invention, detection of a marker in a sample identifies a subject from which the sample was obtained as having a hypersensitivity reaction to the implanted metal.

### Detection Platforms

Methods involving detection and/or quantification of the biomarker of the present invention can be performed using bench-top instruments, or can be incorporated onto disposable, diagnostic or monitoring platforms that can be used in a non-laboratory environment, e.g., in the physician's office or at the patient's bedside.

Thus, as would be understood by one skilled in the art, the methods of the invention may include any method known in the art to detect a biomarker in a sample.

Accordingly, the invention includes any platform for detecting a desired biomarker in a bodily fluid sample such as synovial fluid. In one embodiment, the system of the invention provides a convenient point-of-care device which can quickly detect the presence or absence of a biomarker in an at home or clinical setting. One non-limiting example of a point of care device is a lateral flow immunoassay, which utilizes strips of a membrane, preferably a cellulose membrane, onto which antibodies and other reagents are applied. The sample moves along the strip due to capillary action and reacts with the reagents striped at different points along the strip. The end result is the appearance or absence of a colored line or spot, which optionally can be compared to a control line. In some embodiments the point of care device detects two or more biomarkers. In certain aspects, the two or more biomarkers comprise IL-8 and OPN.

In one embodiment, the system may include a base or support layer and an absorbent matrix comprising at least one absorbent layer through which a liquid sample can flow along a flow path by force or by capillary action. The base layer may also be absorbent and be in fluid communication with the absorbent matrix, such that the flow path of liquid sample passes through both the absorbent matrix and the base layer. The flow path includes at least two regions, where the first region is a sample application region, and the second region is a detection region.

### Point-of-Use Devices

Point-of-use analytical tests have been developed for the routine identification or monitoring of health-related conditions (such as pregnancy, cancer, endocrine disorders, infectious diseases or drug abuse) using a variety of biological samples (such as urine, serum, plasma, blood, saliva). Some of the point-of-use assays are based on highly specific interactions between specific binding pairs, such as antigen/antibody, hapten/antibody, lectin/carbohydrate, apoprotein/cofactor and biotin/(strept)avidin, (receptor/ligand). In some point-of use devices, assays are performed with test strips in which a specific binding pair member is attached to a mobilizable material (such as a metal sol or beads made of latex or glass) or an immobile substrate (such as glass fibers, cellulose strips or nitrocellulose membranes). Other point-of use devices may comprise optical biosensors, photometric biosensors, electrochemical biosensor, or other types of biosensors. Suitable biosensors in point-of-use devices for performing methods of the invention include “cards” or “chips” with optical or acoustic readers. Biosensors can be configured to allow the data collected to be electronically transmitted to the physician for interpretation and thus can form the basis for e-medicine, where diagnosis and monitoring can be done without the need for the patient to be in proximity to a physician or a clinic.

Detection of a biomarker in a bodily fluid (e.g. synovial fluid) can be carried out using a sample capture device, such as a lateral flow device (for example a lateral flow test strip) that allows detection of one or more biomarkers, such as those described herein.

The test strips of the present invention include a flow path from an upstream sample application area to a test site. For example, the flow path can be from a sample application area through a mobilization zone to a capture zone. The mobilization zone may contain a mobilizable marker that interacts with an analyte or analyte analog, and the capture zone contains a reagent that binds the analyte or analyte analog to detect the presence of an analyte in the sample.

Examples of migration assay devices, which usually incorporate within them reagents that have been attached to colored labels, thereby permitting visible detection of the assay results without addition of further substances are found, for example, in U.S. Pat. No. 4,770,853 (incorporated herein by reference). There are a number of commercially available lateral-flow type tests and patents disclosing methods for the detection of small or large analytes (MW smaller or greater than 1,000 Daltons) as the analyte flows through multiple zones on a test strip. Examples are found in U.S. Pat. Nos. 5,229,073, 5,591,645; 4,168,146; 4,366,241; 4,855,240; 4,861,711; 5,120,643 (each of which are herein incorporated by reference). Multiple zone lateral flow test strips are disclosed in U.S. Pat. Nos. 5,451,504, 5,451,507, and U.S. Pat. No. 5,798,273 (incorporated by reference herein). U.S. Pat. No. 6,656,744 (incorporated by reference) discloses a lateral flow test strip in which a label binds to an antibody through a streptavidin-biotin interaction.

Flow-through type assay devices were designed, in part, to obviate the need for incubation and washing steps associated with dipstick assays. Flow-through immunoassay devices involve a capture reagent (such as one or more antibodies) bound to a porous membrane or filter to which a liquid sample is added. As the liquid flows through the membrane, target analyte (such as protein) binds to the capture reagent. The addition of sample is followed by (or made concurrent with) addition of detector reagent, such as labeled antibody (e.g., gold-conjugated or colored latex particle-conjugated protein). Alternatively, the detector reagent may be placed on the membrane in a manner that permits the detector to mix with the sample and thereby label the analyte. The visual detection of detector reagent provides an indication of the presence of target analyte in the sample. Representative flow-through assay devices are described in U.S. Pat. Nos. 4,246,339; 4,277,560; 4,632,901; 4,812,293; 4,920,046; and 5,279,935; U.S. Patent Application Publication Nos. 20030049857 and 20040241876; and WO 08/030,546. Migration assay devices usually incorporate within them reagents that have been attached to colored labels, thereby permitting visible detection of the assay results without addition of further substances. See, for example, U.S. Pat. No. 4,770,853; PCT Publication No. WO 88/08534.

There are a number of commercially available lateral flow type tests and patents disclosing methods for the detection of large analytes (MW greater than 1,000 Daltons). U.S. Pat. No. 5,229,073 describes a semiquantitative competitive immunoassay lateral flow method for measuring plasma lipoprotein levels. This method utilizes a plurality of capture zones or lines containing immobilized antibodies to bind both the labeled and free lipoprotein to give a semi-quantitative result. In addition, U.S. Pat. No. 5,591,645 provides a chromatographic test strip with at least two portions. The first portion includes a movable tracer and the second portion includes an immobilized binder capable of binding to the analyte. Additional examples of lateral flow tests for large analytes are disclosed in the following patent documents: U.S. Pat. Nos. 4,168,146; 4,366,241; 4,855,240; 4,861,711; and 5,120,643; WO 97/06439; WO 98/36278; and WO 08/030,546.

Devices described herein generally include a strip of absorbent material (such as a microporous membrane), which, in some instances, can be made of different substances each joined to the other in zones, which may be abutted and/or overlapped. In some examples, the absorbent strip can be fixed on a supporting non-interactive material (such as nonwoven polyester), for example, to provide increased rigidity to the strip. Zones within each strip may differentially contain the specific binding partner(s) and/or other reagents required for the detection and/or quantification of the particular analyte being tested for, for example, one or more proteins disclosed herein. Thus these zones can be viewed as functional sectors or functional regions within the test device.

In general, a fluid sample is introduced to the strip at the proximal end of the strip, for instance by dipping or spotting. A sample is collected or obtained using methods well known to those skilled in the art. The sample containing the particular proteins to be detected may be obtained from any biological source. In a particular example, the biological source is synovial fluid. The sample may be diluted, purified, concentrated, filtered, dissolved, suspended or otherwise manipulated prior to assay to optimize the immunoassay results. The fluid migrates distally through all the functional regions of the strip. The final distribution of the fluid in the individual functional regions depends on the adsorptive capacity and the dimensions of the materials used.

In some embodiments, porous solid supports, such as nitrocellulose, described elsewhere herein are preferably in the form of sheets or strips. The thickness of such sheets or strips may vary within wide limits, for example, from about 0.01 to 0.5 mm, from about 0.02 to 0.45 mm, from about 0.05 to 0.3 mm, from about 0.075 to 0.25 mm, from about 0.1 to 0.2 mm, or from about 0.11 to 0.15 mm. The pore size of such sheets or strips may similarly vary within wide limits, for example from about 0.025 to 15 microns, or more specifically from about 0.1 to 3 microns; however, pore size is not intended to be a limiting factor in selection of the solid support. The flow rate of a solid support, where applicable, can also vary within wide limits, for example from about 12.5 to 90 sec/cm (i.e., 50 to 300 sec/4 cm), about 22.5 to 62.5 sec/cm (i.e., 90 to 250 sec/4 cm), about 25 to 62.5 sec/cm (i.e., 100 to 250 sec/4 cm), about 37.5 to 62.5 sec/cm (i.e., 150 to 250 sec/4 cm), or about 50 to 62.5 sec/cm (i.e., 200 to 250 sec/4 cm).

Another common feature to be considered in the use of assay devices is a means to detect the formation of a complex between an analyte (such as one or more proteins described herein) and a capture reagent (such as one or more antibodies). A detector (also referred to as detector reagent) serves this purpose. A detector may be integrated into an assay device (for example included in a conjugate pad), or may be applied to the device from an external source.

A detector may be a single reagent or a series of reagents that collectively serve the detection purpose. In some instances, a detector reagent is a labeled binding partner specific for the analyte (such as a gold-conjugated antibody for a particular protein of interest).

In other instances, a detector reagent collectively includes an unlabeled first binding partner specific for the analyte and a labeled second binding partner specific for the first binding partner and so forth. Thus, the detector can be a labeled antibody specific for a protein described herein. The detector can also be an unlabeled first antibody specific for the protein of interest and a labeled second antibody that specifically binds the unlabeled first antibody. In each instance, a detector reagent specifically detects bound analyte of an analyte-capture reagent complex and, therefore, a detector reagent preferably does not substantially bind to or react with the capture reagent or other components localized in the analyte capture area. Such non-specific binding or reaction of a detector may provide a false positive result. Optionally, a detector reagent can specifically recognize a positive control molecule (such as a non-specific human IgG for a labeled Protein A detector, or a labeled Protein G detector, or a labeled anti-human Ab (Fc)) that is present in a secondary capture area.

### Flow-Through Device Construction and Design

A flow-through device involves a capture reagent (such as one or more antibodies) immobilized on a solid support, typically, microtiter plate or a membrane (such as, nitrocellulose, nylon, or PVDF). In a simple representative format, the membrane of a flow-through device is placed in functional or physical contact with an absorbent layer, which acts as a reservoir to draw a fluid sample through the membrane. Optionally, following immobilization of a capture reagent, any remaining protein-binding sites on the membrane can be blocked (either before or concurrent with sample administration) to minimize nonspecific interactions.

In operation of a flow-through device, a fluid sample is placed in contact with the membrane. Typically, a flow-through device also includes a sample application area (or reservoir) to receive and temporarily retain a fluid sample of a desired volume. The sample passes through the membrane matrix. In this process, an analyte in the sample (such as one or more protein, for example, one or more proteins described herein) can specifically bind to the immobilized capture reagent (such as one or more antibodies). Where detection of an analyte-capture reagent complex is desired, a detector reagent (such as labeled antibodies that specifically bind one or more proteins) can be added with the sample or a solution containing a detector reagent can be added subsequent to application of the sample. If an analyte is specifically bound by capture reagent, a characteristic attributable to the particular detector reagent can be observed on the surface of the membrane. Optional wash steps can be added at any time in the process, for instance, following application of the sample, and/or following application of a detector reagent.

### Lateral Flow Device Construction and Design

Lateral flow devices are commonly known in the art. Briefly, a lateral flow device is an analytical device having as its essence a test strip, through which flows a test sample fluid that is suspected of containing an analyte of interest. The test fluid and any suspended analyte can flow along the strip to a detection zone in which the analyte (if present) interacts with a capture agent and a detection agent to indicate a presence, absence and/or quantity of the analyte.

Numerous lateral flow analytical devices have been disclosed, and include those shown in U.S. Pat. Nos. 4,313,734; 4,435,504; 4,775,636; 4,703,017; 4,740,468; 4,806,311; 4,806,312; 4,861,711; 4,855,240; 4,857,453; 4,943,522; 4,945,042; 4,496,654; 5,001,049; 5,075,078; 5,126,241; 5,451,504; 5,424,193; 5,712,172; 6,555,390; 6,258,548; 6,699,722; 6,368,876, 7,517,699 and U.S. patent application Ser. No. 14/971,375 each of which is incorporated by reference.

The test results may be visualized directly, or may be measured using a reader (such as a scanner). The reader device may detect the detection agent as a color, fluorescence, luminescence, radioactivity, or any other detectable marker derived from the labeled reagent from the readout area (for example, the test line and/or control line).

In one embodiment of a lateral flow device, there may be a second (or third, fourth, or more) test line located parallel or perpendicular (or in any other spatial relationship) to test line in test result zone. The operation of this particular embodiment is similar to that described elsewhere herein with the additional considerations that (i) a second detector reagent specific for a second analyte, such as another antibody, may also be contained in the conjugate pad, and (ii) the second test line will contain a second specific binding partner having affinity for a second analyte, such as a second protein in the sample. Similarly, if a third (or more) test line is included, the test line will contain a third (or more) specific binding partner having affinity for a third (or more) analyte.

In one embodiment, a comparison of the control line to the test line yields the test result from the diagnostic system of the invention. In some instances, a valid result occurs when the control line is detected at a higher intensity level than the test line. For example, a valid result occurs when the control line is at least 5% or more, for example, 10%/o, 20%, 30%, 40%, 50%, 60%, 70%, 80%, 90%, 100% or more, darker than the test line. In some instances, a valid result occurs when the control line is at least 0.5 fold or more, for example, 1 fold, 2 fold, 3 fold, 4 fold, 5 fold, 6 fold, 7 fold, 8 fold, 9 fold, 10 fold or more darker than the test line.

In one embodiment, the control line is a reference line that insures that the test has been run correctly and that the tested sample is not obtained from anything other than the joint (i.e. blood). For example, the system of the invention is useful in the diagnosis of ALTR in a joint when the control line is detected at an intensity at least equal to the test line. Preferably, the control line is detected at higher intensity than the test line. In some instances, if the test line is not at least equal in darkness or intensity as the control line then the test is said to have an invalid result. If the test line is at least equal or lighter than the control line then the test is said to have a valid result.

### Point of Care Diagnostic and Risk Assessment Systems

The system of the invention can be applied to a point-of-care scenario. U.S. Pat. Nos. 6,267,722, 6,394,952 and 6,867,051 disclose and describe systems for diagnosing and assessing certain medical risks, the contents of which are incorporated herein. The systems are designed for use on site at the point of care, where patients are examined and tested, as well as for operation remote from the site. The systems are designed to accept input in the form of patient data, including, but not limited to biochemical test data, physical test data, historical data and other such data, and to process and output information, such as data relating to a medical diagnosis or a disease risk indicator. The patient data may be contained within the system, such as medical records or history, or may be input as a signal or image from a medical test or procedure, for example, immunoassay test data, blood pressure reading, ultrasound, X-ray or MRI, or introduced in any other form Specific test data can be digitized, processed and input into the medical diagnosis expert system, where it may be integrated with other patient information. The output from the system is a disease risk index or medical diagnosis.

Point of care testing refers to real time diagnostic testing that can be done in a rapid time frame so that the resulting test is performed faster than comparable tests that do not employ this system For example, the exemplified immunoassay disclosed and described herein can be performed in significantly less time than the corresponding ELISA assay, e.g., in less than half an hour. In addition, point of care testing refers to testing that can be performed rapidly and on site, such as in a doctor's office, at a bedside, in a stat laboratory, emergency room or other such locales, particularly where rapid and accurate results are required.

In an exemplary embodiment, a point of care diagnostic and risk assessment system includes a reader for reading patient data, a test device designed to be read in the reader, and software for analysis of the data. A test strip device in a plastic housing is designed for use with the reader, optionally including a symbology, such as an alphanumeric character bar code, other machine-readable code, or RFID device and software designed for analysis of the data generated from the test strip are also provided.

In one embodiment, a reader refers to an instrument for detecting and/or quantitating data, such as on test strips. The data may be visible to the naked eye, but does not need to be visible. Such readers are disclosed and described in the above-incorporated U.S. Pat. Nos. 6,267,722, 6,394,952 and 6,867,051. A reflectance reader refers to an instrument adapted to read a test strip using reflected light, including fluorescence, or electromagnetic radiation of any wavelength. Reflectance can be detected using a photodetector or other detector, such as charge coupled diodes (CCD). An exemplary reflectance reader includes a cassette slot adapted to receive a test-strip, light-emitting diodes, optical fibers, a sensing head, including means for positioning the sensing head along the test strip, a control circuit to read the photodetector output and control the on and off operation of the light-emitting diodes, a memory circuit for storing raw and/or processed data, and a photodetector, such as a silicon photodiode detector. It will be appreciated that a color change refers to a change in intensity or hue of color or may be the appearance of color where no color existed or the disappearance of color.

In one embodiment, a sample is applied to a diagnostic immunoassay test strip, and colored or dark bands are produced. The intensity of the color reflected by the colored label in the test region (or detection zone) of the test strip is, for concentration ranges of interest, directly proportional or otherwise correlated with an amount of analyte present in the sample being tested. The color intensity produced is read, in accordance with the present embodiment, using a reader device, for example, and a reflectance reader, adapted to read the test strip. The intensity of the color reflected by the colored label in the test region (or detection zone) of the test strip is directly proportional to the amount of analyte present in the sample being tested. In other words, a darker colored line in the test region indicates a greater amount of analyte, whereas a lighter colored line in the test region indicates a smaller amount of analyte. The color intensity produced, i.e., the darkness or lightness of the colored line, is read using a reader device, for example, a reflectance reader, adapted to read the test strip.

A reflectance measurement obtained by the reader device is correlated to the presence and/or quantity of analyte present in the sample. The reader takes a plurality of readings along the strip, and obtains data that are used to generate results that are an indication of the presence and/or quantity of analyte present in the sample. The system may correlate such data with the presence of a disorder, condition or risk thereof.

As mentioned elsewhere herein, in addition to reading the test strip, the reader may be adapted to read a symbology, such as a bar code or RFID device, which is present on the test strip or housing and encodes information relating to the test strip device and/or test result and/or patient, and/or reagent or other desired information. Typically the associated information is stored in a remote computer database, but can be manually stored. Furthermore, the symbology or RFID device can be imprinted when the device is used and the information encoded therein.

### Kit

The invention includes a set of preferred antibodies or oligonucleotides, either labeled (e.g., fluorescer, quencher, etc.) or unlabeled, that are useful for the detection of at least one biomarker of the present invention.

In certain embodiments, a kit is provided. Commercially available kits for use in these methods are, in view of this specification, known to those of skill in the art. In general, kits will comprise a detection reagent that is suitable for detecting the presence of a biomarker (polypeptide or nucleic acid, or mRNA) of interest.

In one embodiment, a kit is provided with instructions for use wherein the kit comprises an antibody or a probe capable of binding to or hybridizing to at least one biomarker. The biomarker may for example, be selected from the group consisting of Neutrophil defensin 1, C-reactive protein, Growth-regulated alpha protein, Neutrophil elastase, Interferon gamma, Interleukin 1-alpha, Interleukin 1-beta, Interleukin 6, Interleukin 8, Interleukin 12-beta, Interleukin 15, C-X-C motif chemokine 10, Lactate, Leptin, Monocyte chemotactic protein 1, Monocyte chemotactic protein 3, C-C motif chemokine 22, Macrophage inflammatory protein 1-alpha, Tumor necrosis factor receptor superfamily member 11B, Osteopontin; Platelet-derived growth factor subunit B, Pentraxin-3, Tumor necrosis factor alpha, Vascular endothelial growth factor, Tumor necrosis factor ligand superfamily member 6 and Soluble intercellular adhesion molecule-1. In some embodiments, the kit is useful for measuring the level of ALTR in a bodily fluid sample from a test subject. In other embodiments, the kit is useful for providing indication of presence or absence of ALTR In other embodiments, the kit is useful for differentiating between ALTR and PJI. In other embodiments the kit is useful for diagnosing immune hypersensitivity to the metal implant. In yet other embodiments, the kit is useful for providing recommendation of whether or not revision surgery would be relevant for the test subject.

In some embodiments, the kit includes a panel of probe sets or antibodies. In some embodiments, the kit is an immunoassay kit as described previously herein. In some embodiments, probe sets are designed to detect the level of at least one biomarker of the present invention and provide information about the ALTR or tissue necrosis. In the present invention, the probe sets are targeted to the detection of nucleotides or polypeptides that are informative about ALTR. Probe sets may also comprise a large or small number of probes that detect nucleotides or peptides that are not informative about ALTR or tissue necrosis. Such probes are useful as controls and for normalization (e.g., spiked-in markers). Probe sets may be a dry mixture or a mixture in solution. In some embodiments, probe sets can be affixed to a solid substrate to form an array of probes. The probes may be antibodies, or nucleic acids (e.g., DNA, RNA, chemically modified forms of DNA and RNA), LNAs (Locked nucleic acids), PNAs (Peptide nucleic acids), antibody-nucleic acid conjugates, or any other polymeric compound capable of specifically interacting with the peptides or nucleic acid sequences of interest. A device for measurement of the biomarkers of the present invention may also be included in the kit.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The materials and methods employed in the experiments disclosed herein are now described.

### Materials and Methods

### Patient Selection and Treatment Plan

Patients undergoing a revision metal-on-metal (MOM) or metal-on-poly (MOP) total hip replacement or a primary total hip replacement were screened for eligibility based on the inclusion and exclusion criteria listed.

### Inclusion Criteria:

1. Patients presenting for one of the following procedures:
   a. A metal on metal hip revision where the patient has cobalt and chromium metal ion levels tested within 6 months of the date of the planned revision surgery. OR
   b. Patients presenting for a metal on poly hip revision OR
   c. Patients who have hip osteoarthritis but have not had a total hip surgery (control)
2. Revision Procedures only: Patients must be greater than one year postoperative

### Exclusion Criteria:

1. Primary Procedures only: Patients with a total hip of any articulation on the contralateral side
2. Patients with a prior history of periprosthetic infection
3. Prisoners
4. Patients not willing to consent for the proposed treatment
5. Patients with an altered mental status
6. Active, concurrent metastatic infection
7. Active, superficial infection
8. Patients who have had a preoperative synovial fluid aspiration within 2 weeks (14 days) of the scheduled procedure
9. Metal-on-Metal (MOM) Revision Procedures only: Patients presenting for a metal on metal hip revision who have cobalt and chromium metal ion levels tested >6 months of the date of the planned revision
10. Metal-on-Poly (MOP) Revision Procedures only: Patients presenting for a metal on poly hip revision to treat trunionosis
11. Revision Procedures only: Patients with a metal-on-metal total hip on the contralateral side

### Primary/Secondary Outcome Variables

Primary Endpoint: Serum and synovial fluid biomarker(s) that predicts ALTR necrosis
Secondary Endpoints:
   Association of positive biomarker result with an intraoperative ALTR using surgeon and histologic grading systems. See Appendix 2 for scoring systems
   Association between preoperative cobalt and chromium ion levels and intraoperative ALTR using surgeon and histologic grading systems

### Treatment Administration

### Preoperative Visit:

At the preoperative visit patients will be screened based on medical history and inclusion/exclusion criteria. Patients meeting the criteria are asked to volunteer their participation. The patients who agree to participate are asked to sign an informed consent form before any study data is collected or study procedures are performed.

A blood sample is taken from patients preoperatively to test for various serum biomarkers. Two tubes of blood (approximately 8.0 mL, or about 2 teaspoon) are drawn into a 4 mL red top (clot activator) blood collection tube. Tubes are gently inverted 5-6 times and allowed to clot for a minimum of 30 minutes (or until clotting is visible). Tubes are centrifuged within 1 hour of collection at 1,300 g for 15 minutes at room temperature. Each separated serum sample is aspirated with a sterile pipette and transferred into two (or more) cryovials. Each cryovial should contain a minimum of 0.5 mL of serum If there is less than 1 mL of serum after centrifuging the specimen for 15 minutes, centrifugation is repeated at 1,300 g for an additional 10 minutes. The remaining serum is placed in one or more cryovials.

All serum tubes are labeled with a de-identified study ID number and stored at a minimum of −50 qC or less until shipment to the lab. The freezer temperature is recorded at least daily to ensure that the samples are maintained at a minimum of −50° C. One cryovial sample is retained at the site; all other cryovials are shipped to the lab on dry ice upon the lab's request. The lab is notified and frequently updated on patient enrollment. The retention samples are kept in the freezer until the end of the study or until the lab requests they be shipped or destroyed. The master list with patient names and corresponding study ID numbers is maintained by the research staff and is not shared with any outside parties.

The Following Data is Collected from all Patients:
Eligibility Screening
Patient demographics
Patient medical history
Intraoperative complications
   The Following Additional Data is Collected from Metal-On-Metal Revision Patients Only:
Histology Assessment
Intraoperative tissue damage
Pathology Reports including microscopic histology of tissues
Routine preoperative Chromium-Cobalt laboratory results

### Operative Visit:

In the operating room at the beginning of the surgery, the patient's surgeon draws a sample of synovial fluid from the operative hip. The sample is split equally between two red top sample tubes without clot activator. All tubes are labeled with a de-identified study ID number and stored at a minimum of −20° C.

One sample tube is centrifuged for 10 minutes at 1000 g. The supernatant is aliquoted into a minimum of four 500 uL cryovials. If a significant quantity of fluid is obtained that yields >10 vials, the fluid is distributed equally between the 10 vials. All cryovials from the specimen that was centrifuged is labeled with a de-identified study ID number and the letter “S” for spun.

The second sample is aliquoted into a minimum of four 500 uL cryovials without centrifugation. If a significant quantity of fluid is obtained to that yields greater than 10 vials, the fluid is distributed equally between the 10 vials. All cryovials from the specimen that was not centrifuged are labeled with a de-identified study ID number and the letter “U” for unspun

An example of specific biomarkers that were tested is listed in Table 1 below.

TABLE 1

| ID | Biomarker |
|---|---|
| 1 | 8-oxoguanine |
| 2 | Active caspase8 |
| | (ASP 384) |
| 3 | Active caspase 9 |
| | (ASP 315) |
| 4 | Akt (Ser473) |
| 5 | Amylin (active) |
| 6 | Amylin (total) |
| 7 | BAD (Ser 112) |
| 8 | Bcl-2 (Ser 70) |
| 9 | BPI |
| 10 | Caspase 3 (active) |
| 11 | CC16 |
| 12 | C-Peptide |
| 13 | CRP |
| 14 | EGF |
| 15 | ELA |
| 16 | eNOS |
| 17 | Eotaxin |
| 18 | FAS Ligand |
| 19 | FGF-2 |
| 20 | Fibrinogen |
| 21 | Flt-3 Ligand |
| 22 | Fractalkine |
| 23 | GAPDH (total) |
| 24 | G-CSF |
| 25 | Ghrelin (active) |
| 26 | GIP (total) |
| 27 | GLP-1 (active) |
| 28 | Glucagon |
| 29 | GM-CSF |
| 30 | Granzyme B |
| 31 | GRO |
| 32 | HIF 1 alpha |
| 33 | HSP70 |
| 34 | ICAM-1 |
| 35 | IFN-α2 |
| 36 | IFN-γ |
| 37 | IL-10 |
| 38 | IL-12 (p40) |
| 39 | IL-12 (p70) |
| 40 | IL-13 |
| 41 | IL-15 |
| 42 | IL-17 |
| 43 | IL-Rα |
| 44 | IL-1α |
| 45 | IL-1β |
| 46 | IL-2 |
| 47 | IL-3 |
| 48 | IL-4 |
| 49 | IL-5 |
| 50 | IL-6 |
| 51 | IL-7 |
| 52 | IL-8 |
| 53 | IL-9 |
| 54 | IP-10 |
| 55 | JNK |
| 56 | KI M1 |
| 57 | Lactoferrin |
| 58 | Leptin |
| 59 | MCO-1 |
| 60 | MCP-1 |
| 61 | MCP-3 |
| 62 | MDC (CCL22) |
| 63 | MIF |
| 64 | MIP-1α |
| 65 | MIP-1β |
| 66 | MMP-8 |
| 67 | NF-Kb |
| 68 | NGAL |
| 69 | p53 |
| 70 | PAI-1 |
| 71 | PARP (cleaved) |
| 72 | PDGF-AA |
| 73 | PDGF-AB/BB |
| 74 | PP |
| 75 | PYY |
| 76 | RANTES |
| 77 | Resistin |
| 78 | sCD40L |
| 79 | sIL-2R |
| 80 | sTNF-RII |
| 81 | TGF-α |
| 82 | Thrombospondin |
| 83 | Tissue factor |
| 84 | TNT-α |
| 85 | TNF-β |
| 86 | VCAM-1 |
| 87 | VEGF |
| 88 | α-defensin |
| 89 | ACTH |
| 90 | DKK1 |
| 91 | FGF-23 |
| 92 | Insulin |
| 93 | OC |
| 94 | OPN |
| 95 | OPG |
| 96 | PTH |
| 97 | SOST |
| 98 | RANKL |
| 99 | TRAP5b |
| 100 | Cathepsin K |
| 101 | BAP |
| 102 | Caspase-1 |
| 103 | IL-33 |
| 104 | Cyclophilin A |
| 105 | FABP3 |
| 106 | PGE2 |
| 107 | IL-18 |
| 108 | TRAP5a |
| 109 | CTX-1 |
| 110 | ADA |
| 111 | BAFF |
| 112 | April |
| 113 | CCL21 |
| 114 | CXCL13 |
| 115 | CAIX |
| 116 | Transthyretin |
| 117 | GSN |
| 118 | Lactate |
| 119 | Fas |
| 120 | MPO |
| 121 | MAP-3 |
| 122 | MMP-12 |
| 123 | MMP-13 |
| 124 | MMP-1 |
| 125 | MMP-2 |
| 126 | MMP-7 |
| 127 | MMP-9 |
| 128 | MMP-10 |
| 129 | MRP 8 |
| 130 | c-Myc |
| 131 | FADD |
| 132 | IκBα |
| 133 | IKKα/β |
| 134 | NF-κb |
| 135 | TNFRI |
| 136 | IL-21 |
| 137 | SOD2 |
| 138 | SOD1 |
| 139 | CATALASE |
| 140 | MDM2 |
| 141 | IGF1 |
| 142 | IGF2 |
| 143 | B2M |
| 144 | PTX3 |
| 145 | TF |
| 146 | APOA1 |
| 147 | APOC3 |
| 148 | SAA |
| 149 | ANTITHROMBIN |
| | III |
| 150 | ECM1 |
| 151 | Fibroblast |
| | Activation Protein |
| 152 | GALECTIN3 |
| 153 | VITRONECTIN |
| 154 | BMP9 |
| 155 | FGF1 |
| 156 | KERATIN 1,10 |
| 157 | FIBRONECTIN |
| 158 | INVOLUCRIN |
| 159 | KERATIN 6 |
| 160 | CO4 |
| 161 | HP |
| 162 | SAP/SERUM |
| | AMYLOID P |
| 163 | A2M |
| 164 | CATHEPSIN D |
| 165 | C3 |
| 166 | TGFβ1 |
| 167 | TGFβ2 |
| 168 | Granzyme a |
| 169 | ITAC |
| 170 | IL-23 |
| 171 | TIMP1 |
| 172 | TSLP |
| 173 | HMGB1 |
| 174 | sCD163 |
| 175 | SOD3 |
| 177 | BMP4 |
| 178 | GPX3 |
| 179 | CEBPB |
| 180 | HRG |
| 181 | PROTHROMBIN |
| | (F2) |
| 182 | PLG |
| 183 | CHI3L1 |
| 185 | RIPK3 |
| 186 | RIPK1 |
| 187 | NRAM1 |
| 188 | THIO |
| 189 | CD8A |

All cryovials were stored at a minimum of −50° C. or less until shipment to the lab. The treating surgeon noted any intraoperative complications.

The treating surgeon completed the intraoperative tissue damage score (Griffin et al., J Arthroplasty. 2012; 27(8 Suppl):32-6) and collected routine tissue samples to send to the histology lab. A pathologist completed the histology assessment.

The results of the experiments are now described in the following examples.

### Example 1: Validated Necrosis Assay to Determine Need for Revision Surgery in MOM Total Hip Arthroplasty

A study was designed to develop a biomarker assay to be used as a diagnostic tool for ALTR in a subject having a MOM total hip replacement (THR).

To identify biomarkers of ALTR it is necessary to compare the composition of samples from patients with ALTR to those of patients in different disease categories. Molecules, typically proteins, present in patients with ALTR and no other disease groups or normal individuals, are potential biomarkers of ALTR. A critical aspect of a biomarker discovery program is acquisition and testing of well-characterized patient samples from multiple disease categories.

The study was a multicenter, prospective cohort study. To maximize the chance of success of identifying serum biomarkers, all samples were analyzed using multianalyte assay, biomarker tests. A primary endpoint of the study included identifying serum and synovial fluid biomarker(s) that predicts ALTR necrosis. Secondary endpoints included determining association of positive biomarker result with an intraoperative ALTR using surgeon and histologic grading systems.

An important aspect of this study is that the MOM samples are characterized with respect to blood metal ion concentration, metal staining, the amount of extracapsular fluid, histological necrosis and ALVAL score including, descriptions of the synovial lining, the nature of any inflammatory infiltrate and overall tissue organization. The extensive characterization of the patients and samples enables disease-specific biomarker identification.

### Example 2: Target Biomarkers

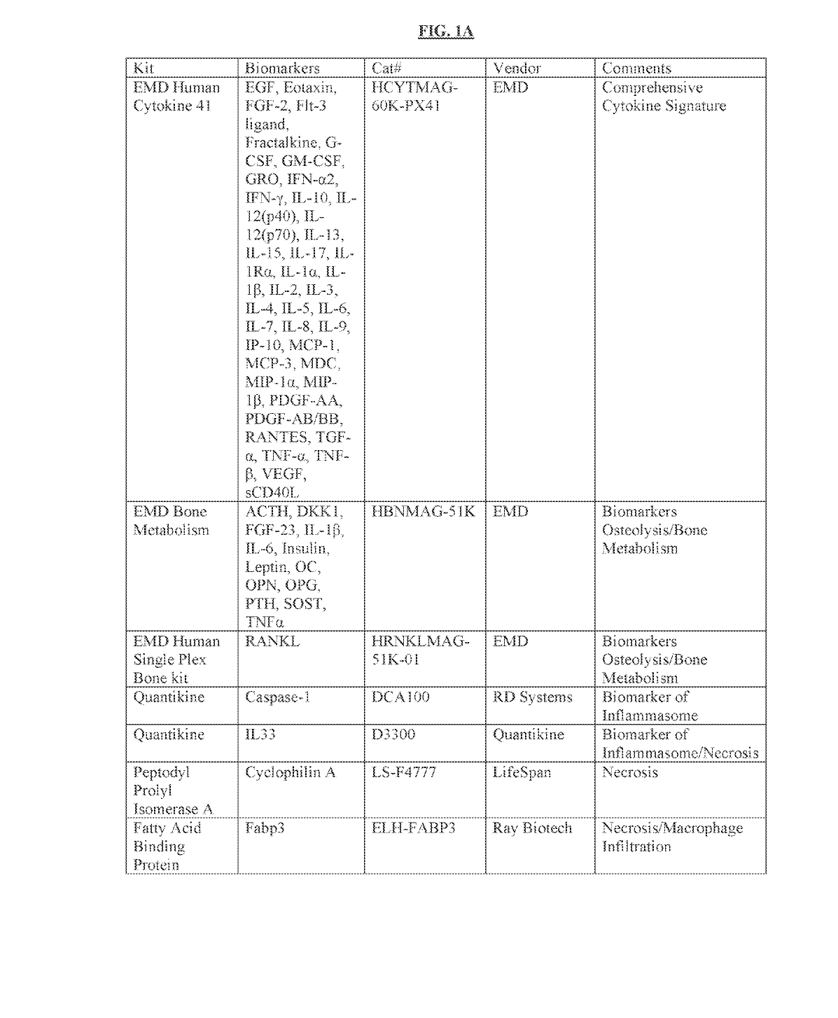

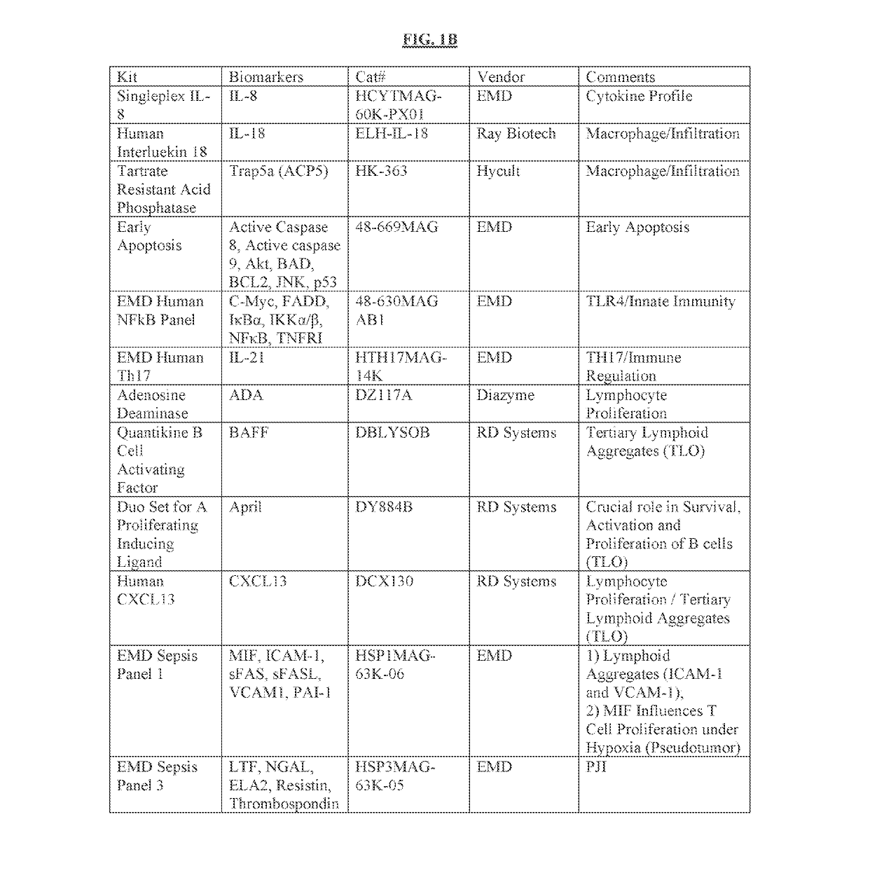

Biomarker proteins were selected for analysis if they were described or suspected to be involved in the molecular mechanisms underlying the observed histological pathology of ALTR In particular, biomarkers of macrophages, lymphocytes, T-cell mediated immunity, delayed-type hypersensitivity, innate immunity, necrosis, apoptosis, cell proliferation, osteolysis, wound healing, bone remodeling, and oxidative stress were selected. Biomarkers of general inflammation and PJI were also included. In total, 8 multiplex Luminex assays were used to analyze 82 different biomarkers. Additionally, 17 biomarkers were analyzed using individual ELISA and enzyme assays (FIGS. 1A-1C).

### Example 3: Primary Biomarkers Screening

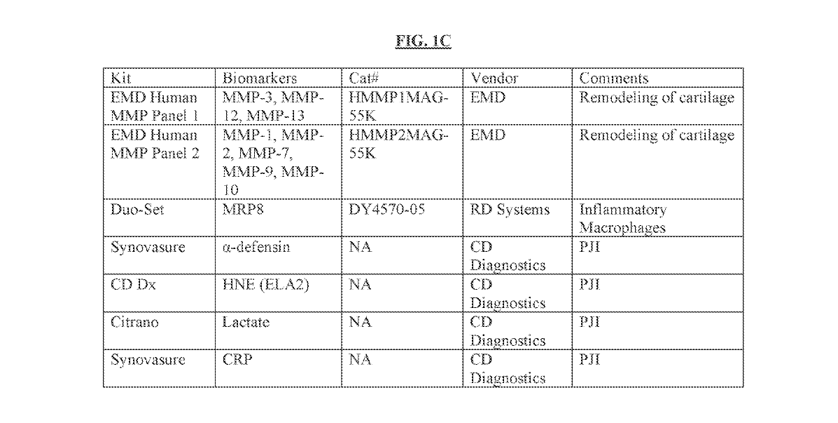

The process of identifying biomarkers of ALTR started with assembly of a relatively small number of synovial fluid samples to be analyzed for a large number of target biomarkers (99). All assays were performed according to manufacturing recommendations with minor modifications. In addition to MOM samples, pooled synovial fluid samples from patients with PJI, OA, and aseptic joints were used as controls (FIG. 2).

In the primary screening campaign, pools of synovial fluid samples were prepared from patients with aseptic joints (3 aseptic samples were pooled together), osteoarthritis (OA) (5 OA samples were pooled together), and PJI (4 PJI samples were pooled together), and analyzed alongside 5 individual MOM samples in assays for 99 human biomarkers. As used herein, an aseptic sample is taken from a subject with a low probability of ALTR or PJI; an OA sample is taken from an OA subject that has not undergone joint replacement surgery and a PJI sample is taken from a subject that has undergone joint replacement surgery and has a demonstrated microbiological infection, and a MOM sample is taken from a subject with a symptomatic/painful MOM joint implant that is not caused by an infection. All samples were stored frozen prior to analysis. Thawed samples (200 ul) were treated with hyaluronidase (20 ul with a concentration of 10 mg/ml) to reduce the viscosity of the synovial fluid, filtered and centrifuged through a 0.2 um membrane and diluted in assay buffer (1:3 to 1:100) prior to testing.

The large majority of all of the methods used herein included standard curves for determining the concentration of the biomarker in the sample, however, none of the kits were validated specifically for use with synovial fluid. The synovial fluid concentrations of most of the biomarkers analyzed in each of the 4 disease categories (aseptic, OA, PJI and MOM) were unknown. Different dilutions of each sample in buffer were assayed to increase the probability of testing a concentration within the working range of the method and to assess the effect of the synovial fluid matrix on assay performance. If the biomarker concentration in the sample was lower than the lowest standard, the result was reported as ‘out-of-range low’ (OOR<). In some assays the biomarker concentrations were above the highest standard and were either further diluted and re-run or, reported as ‘out-of-range high’ (OOR>). The quality of the data generated from each test method was judged based on the agreement between biomarker concentrations determined using different sample dilutions, precision of replicate measurements, and background calculated values of the standard curve.

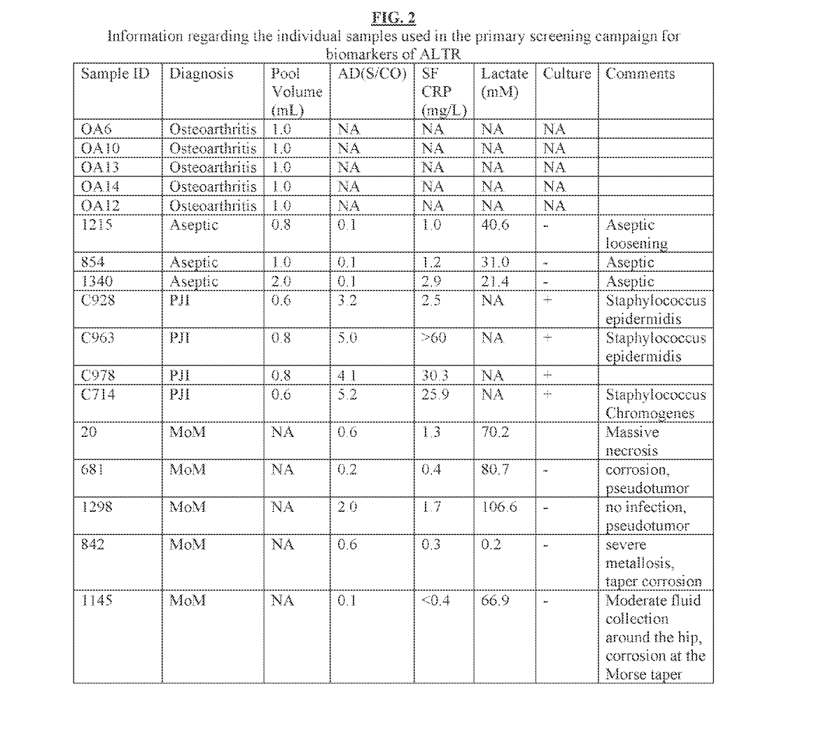

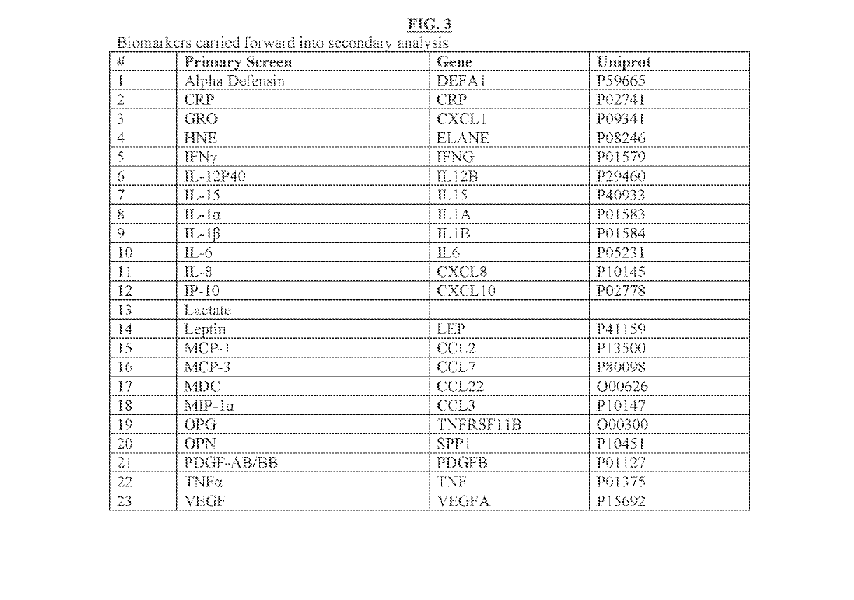

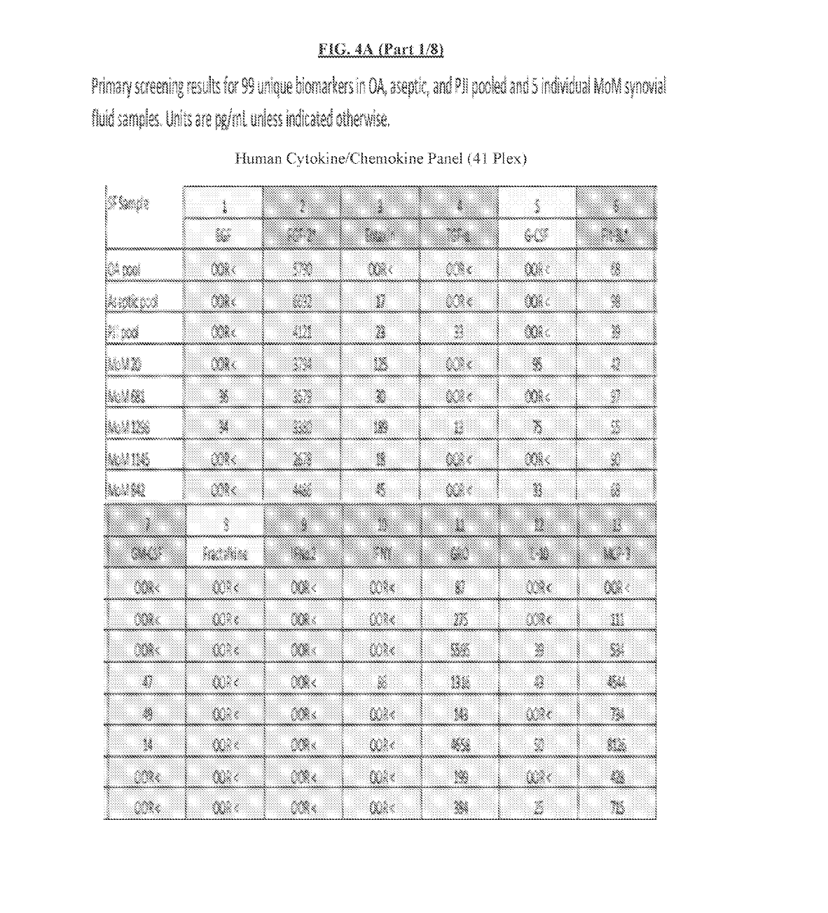

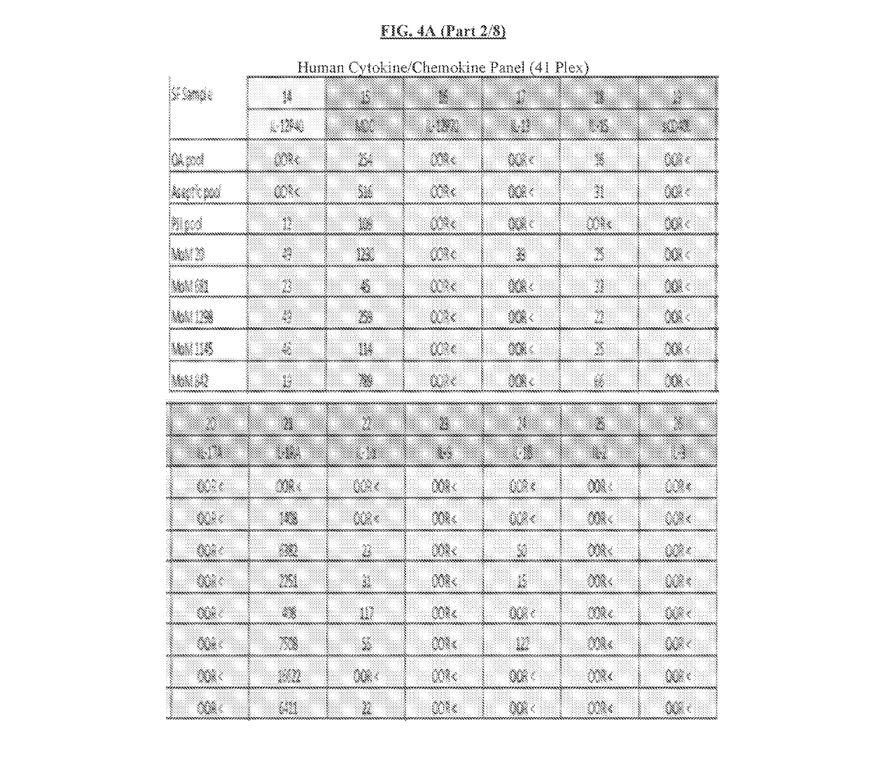

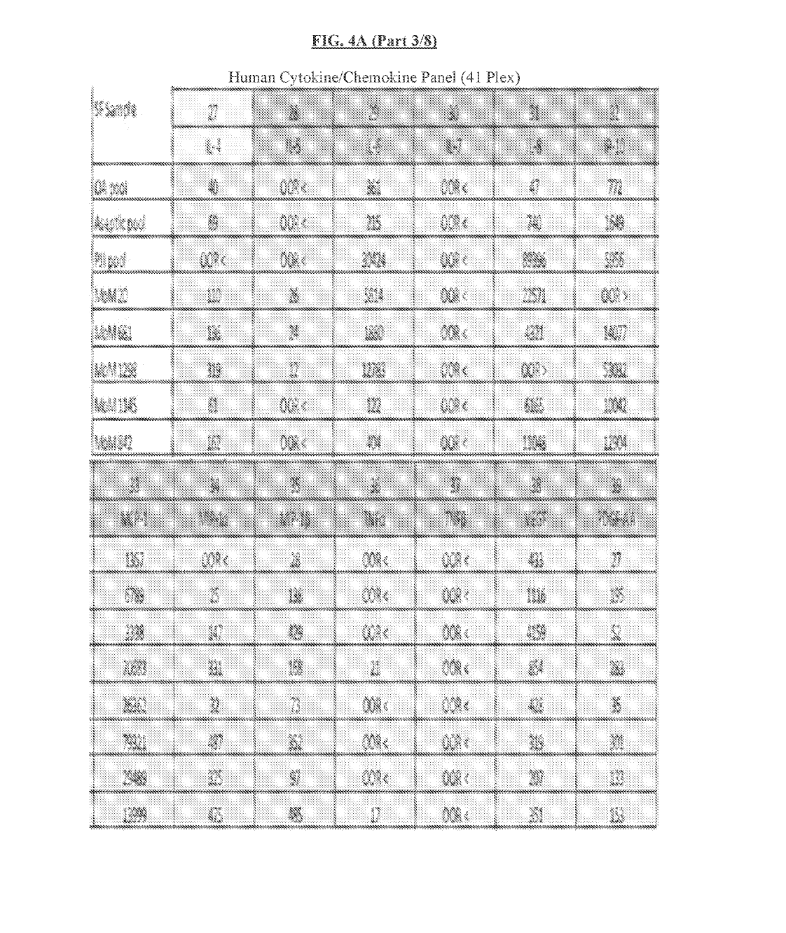

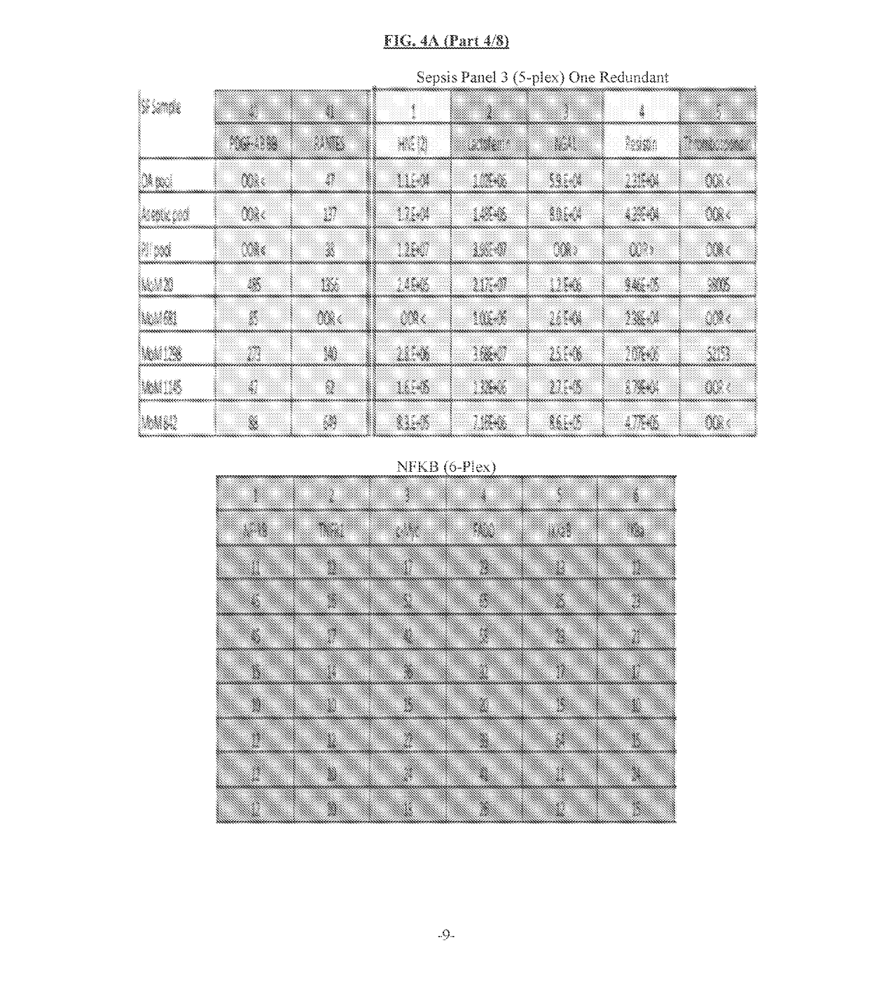

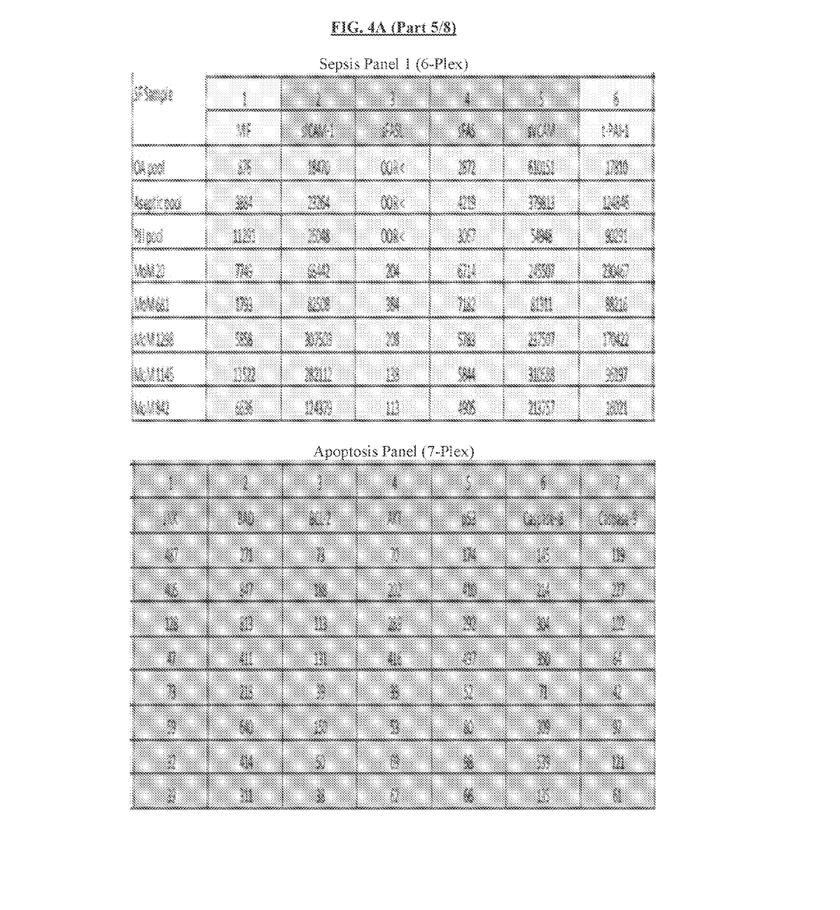

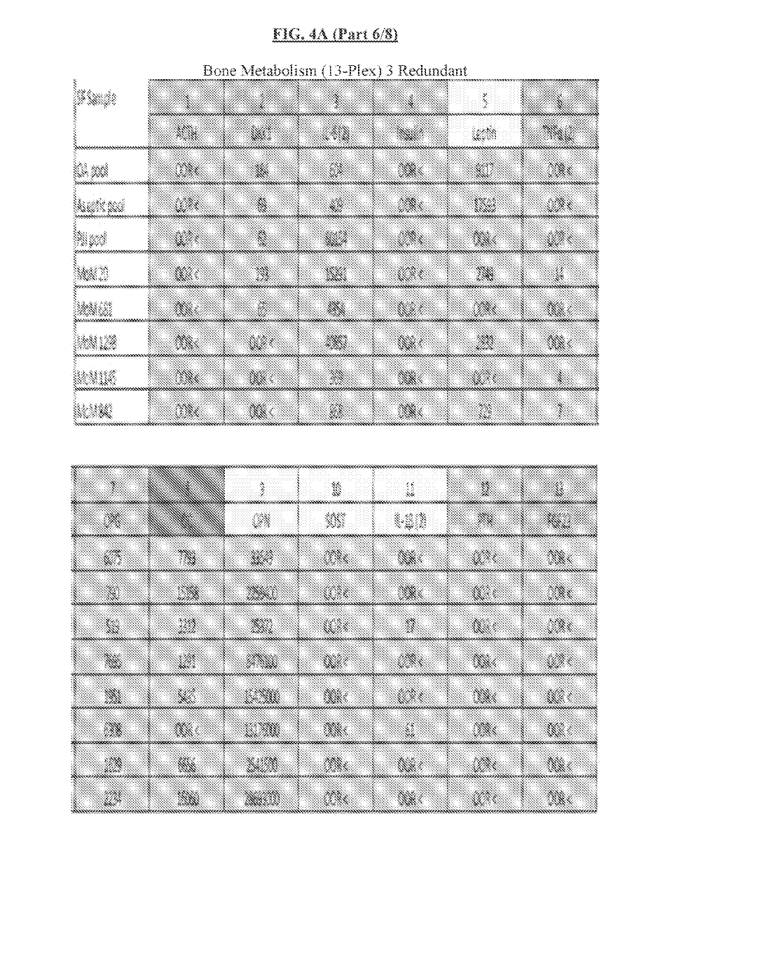

In the primary screen, approximately one-third of the 99 biomarkers were negative with the majority of the samples tested. Notably, IFNγ, TNF and IL-12 were all negative. While other researchers have reported similar findings for these biomarkers in synovial fluid, the results are surprising because these biomarkers are the hallmarks of delayed-type hypersensitivity reactions, one of the disease mechanisms believed by many researchers to be a primary underlying cause of ALTR. Biomarkers showing differences between MOM and the 3 control pooled samples by visual inspection of the data were selected for secondary analysis (FIGS. 3, 4A(Part 1/8) through 4A(Part 8/8), and 4B).

### Example 4: Secondary Biomarkers Screening

The Secondary Biomarkers Screening involved fewer biomarkers (23) and much larger numbers of samples (68) using the same assays as those used in the primary screening. Testing larger numbers of individual samples of each disease group enabled ROC curve analysis of the data to establish cutoff concentrations between groups as well as clinical sensitivity and specificity. For each biomarker, some of the 68 samples contained concentrations within the working range of the assay and some contained concentrations that were outside the range, either high or low. Samples that were out of range were assigned a concentration equal to the sample dilution factor multiplied by the concentration of the low standard or the high standard depending on whether the sample concentration was out of range low or high respectively. Cutoff concentrations were calculated by ROC curve analysis comparing both the aseptic controls and the MOM data and, the data from all 3 control groups (aseptic, OA and PJI) and the MOM samples.

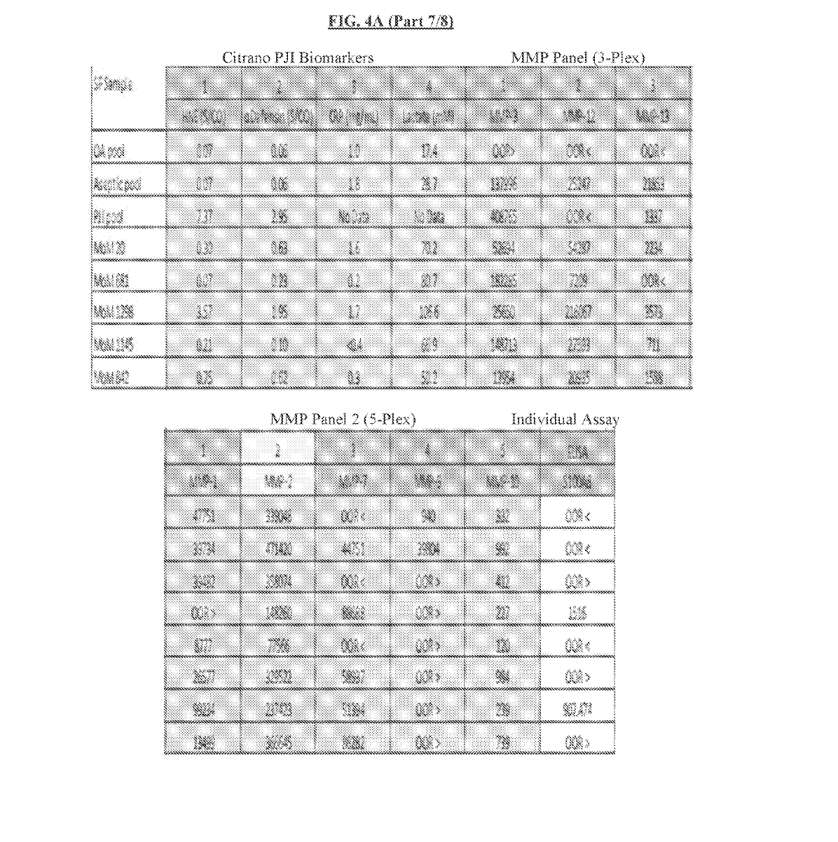

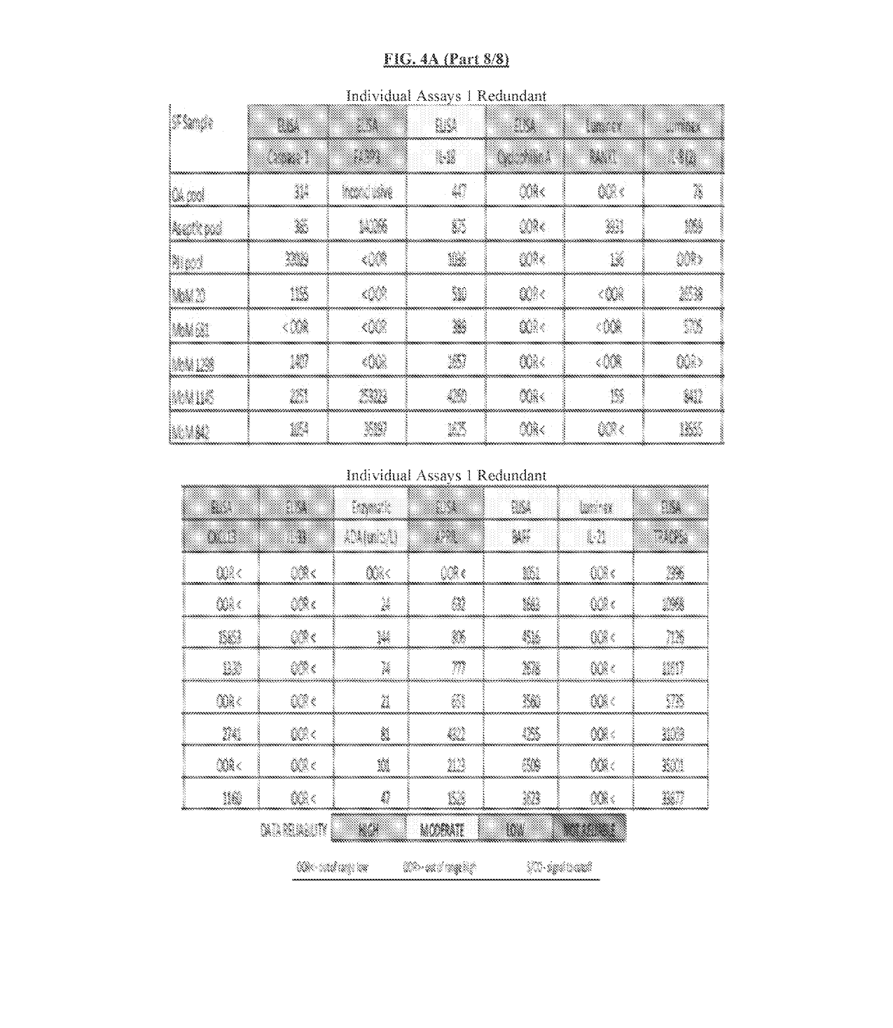

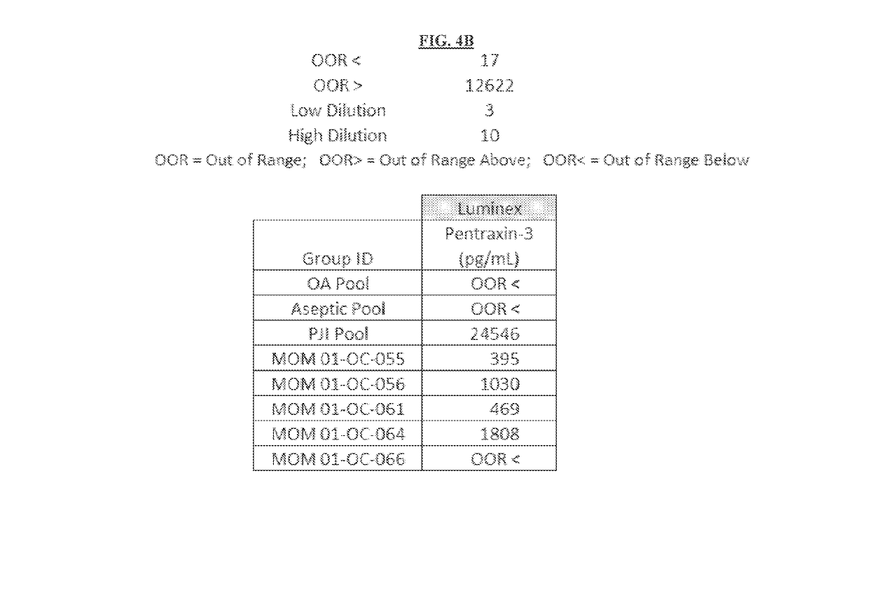

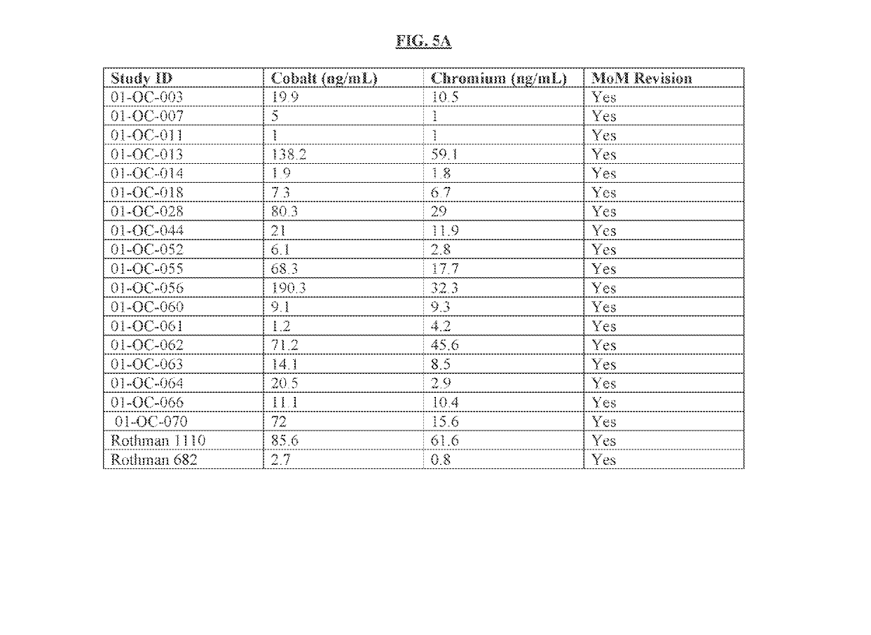

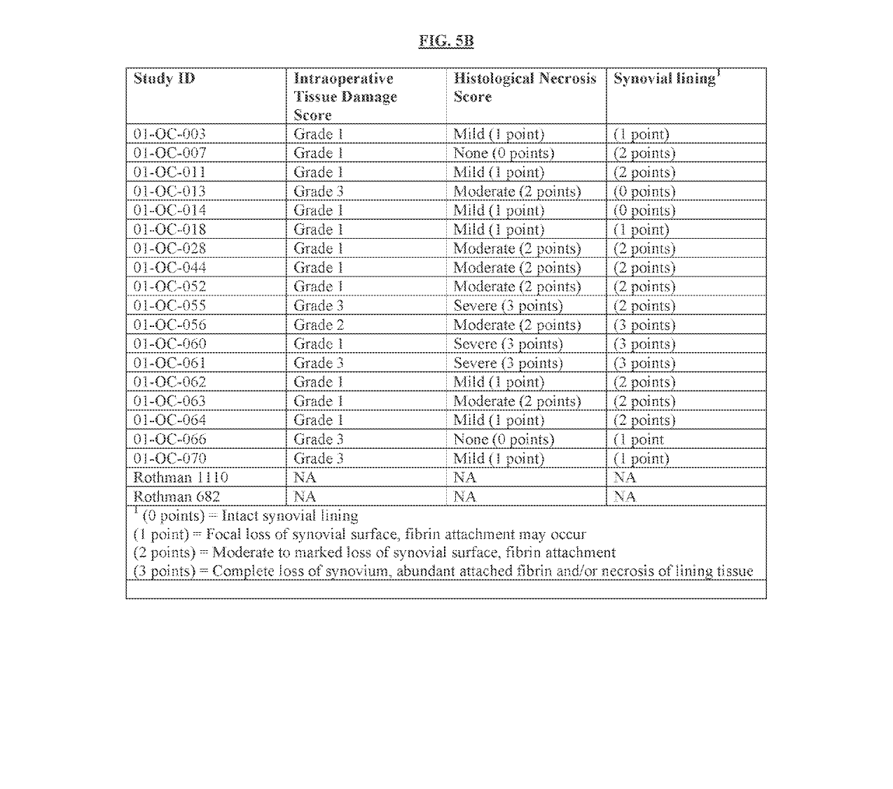

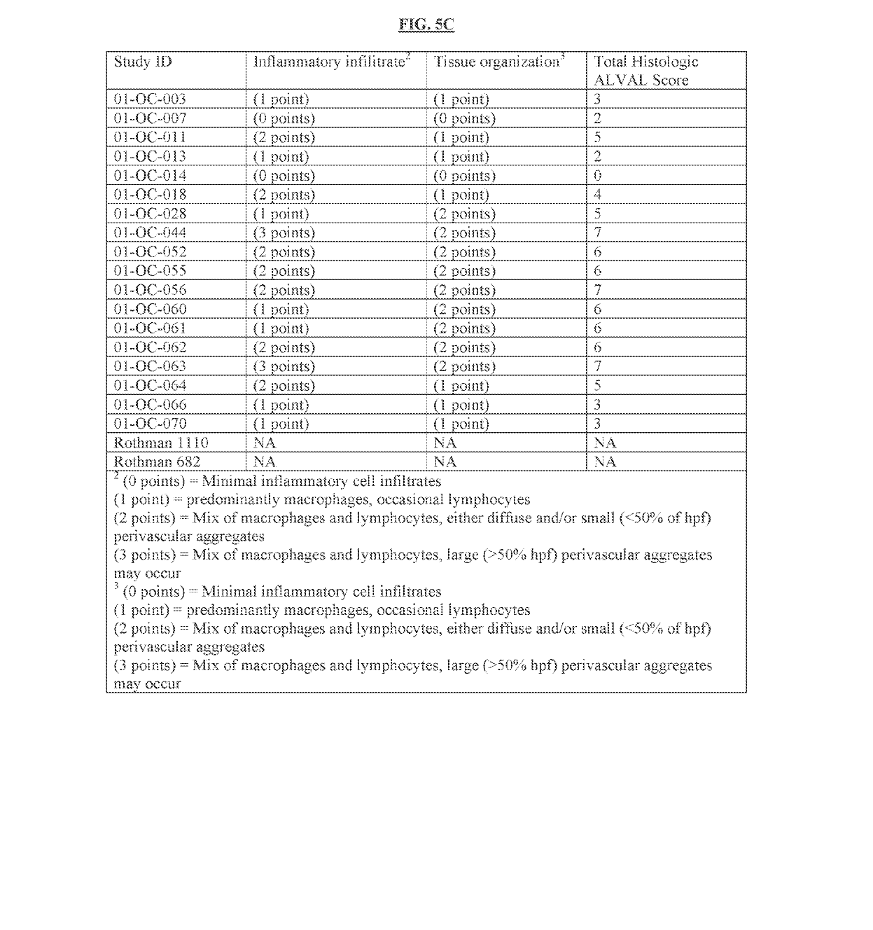

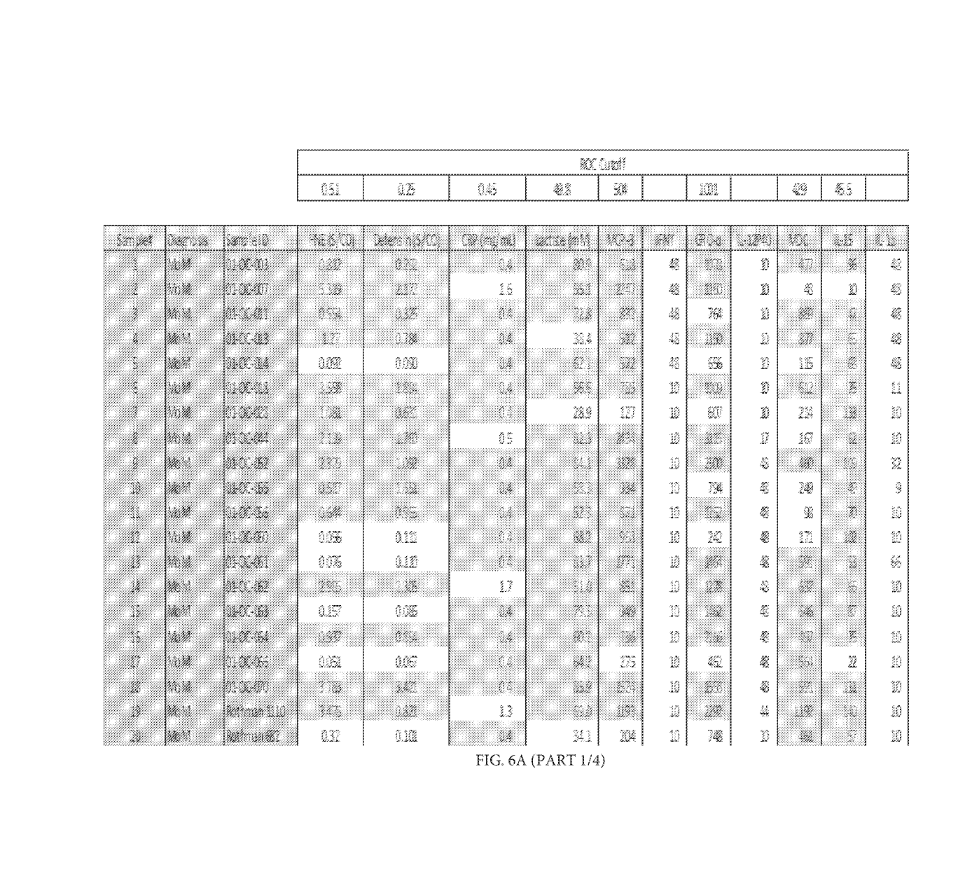

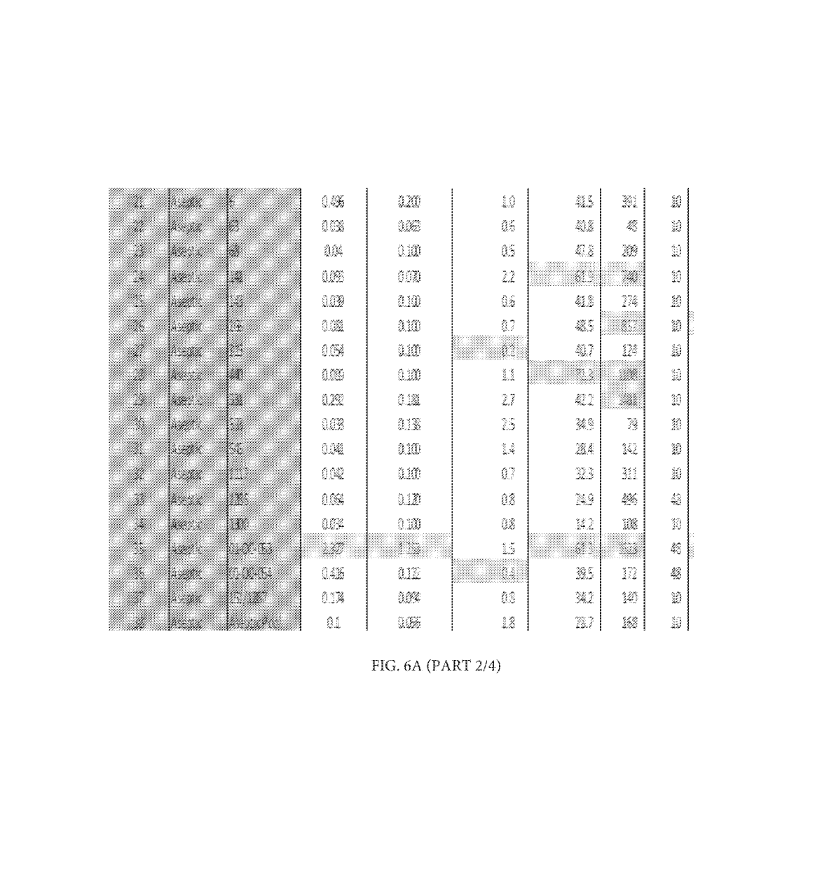

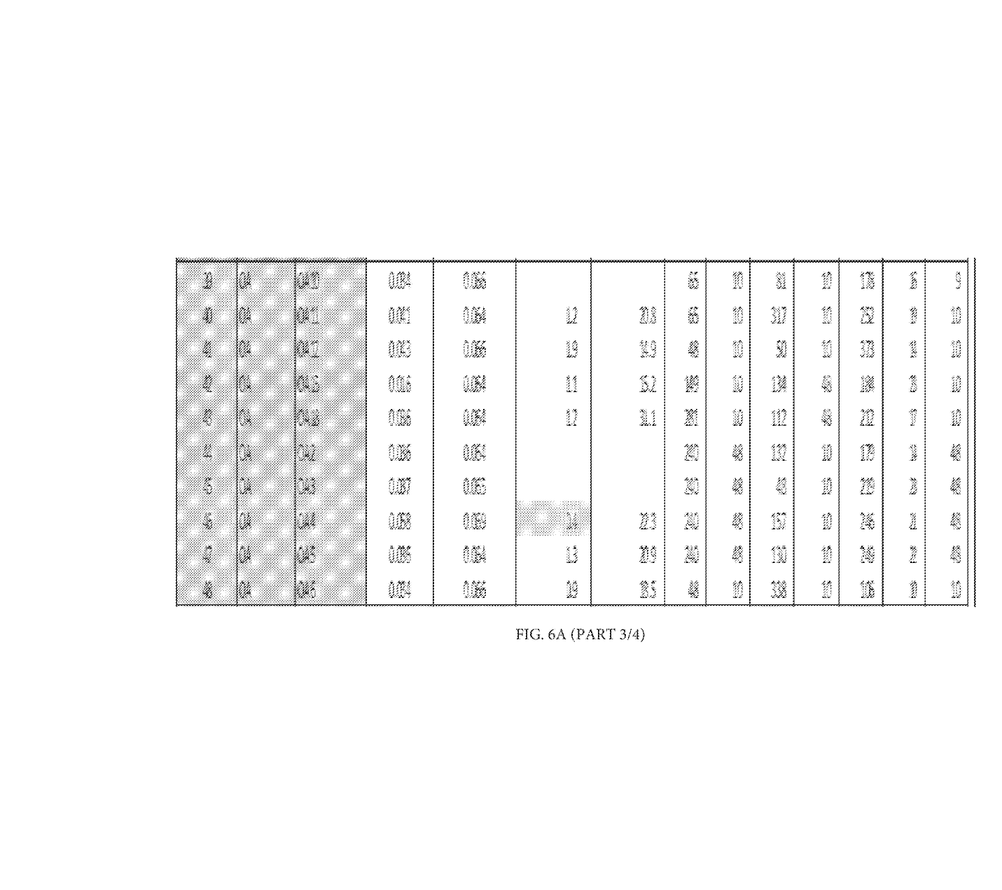

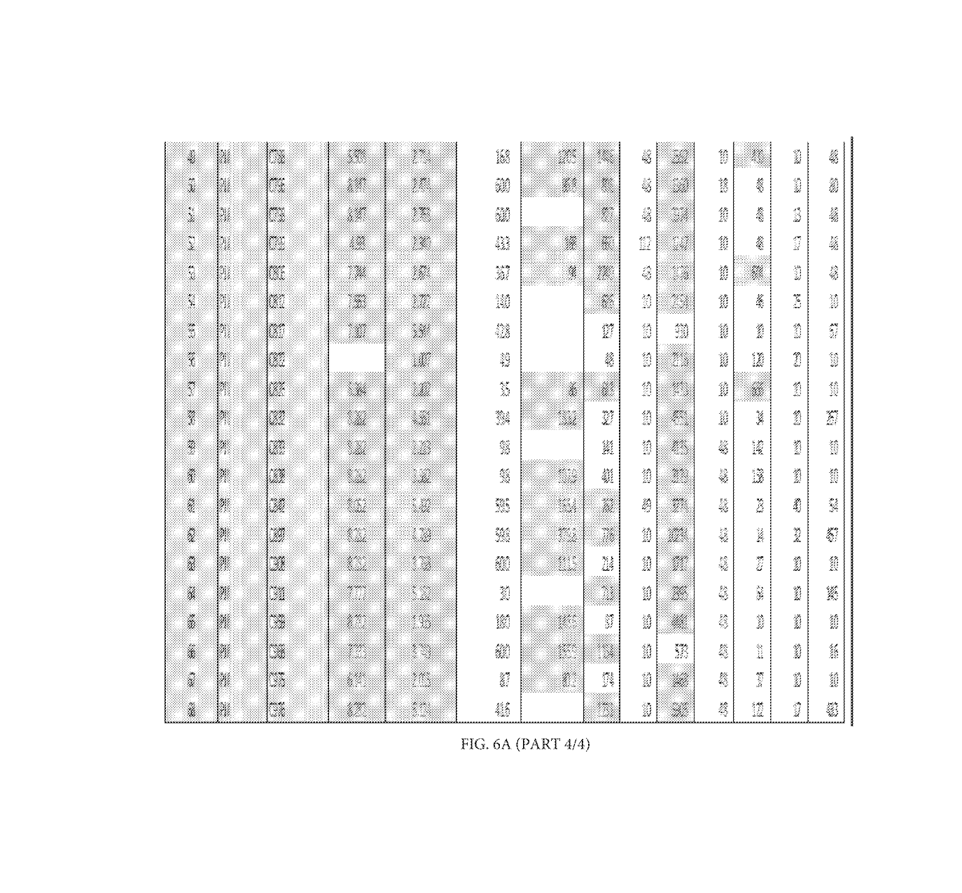

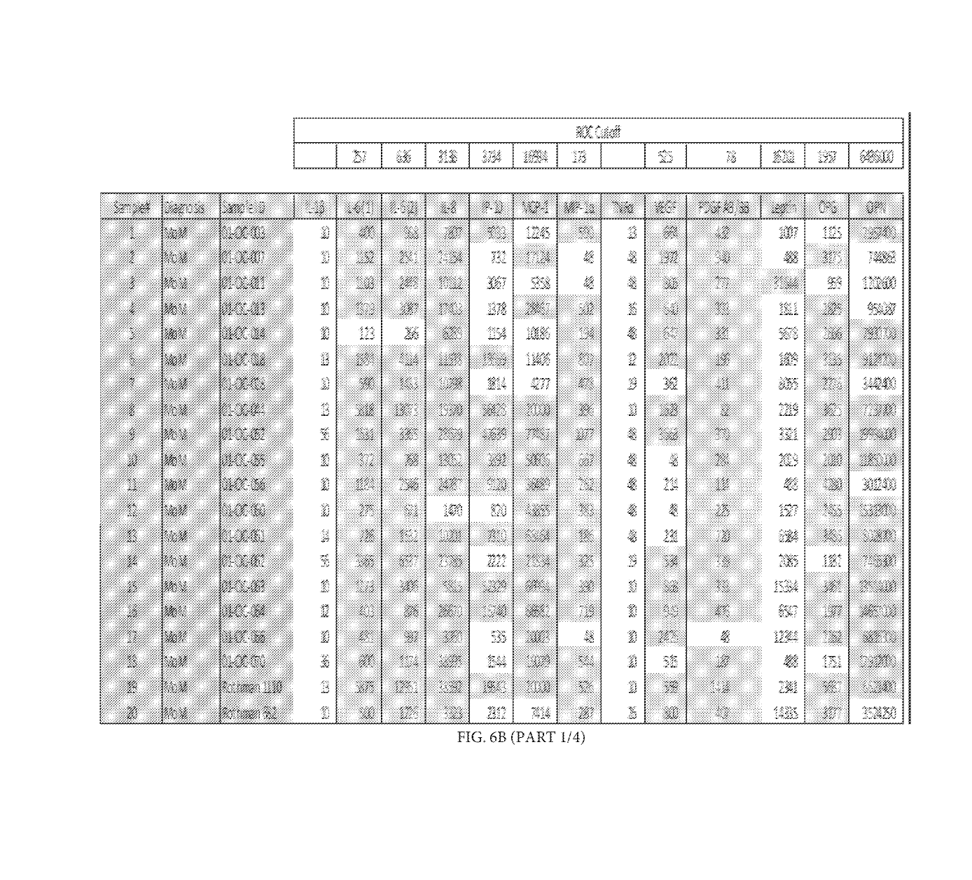

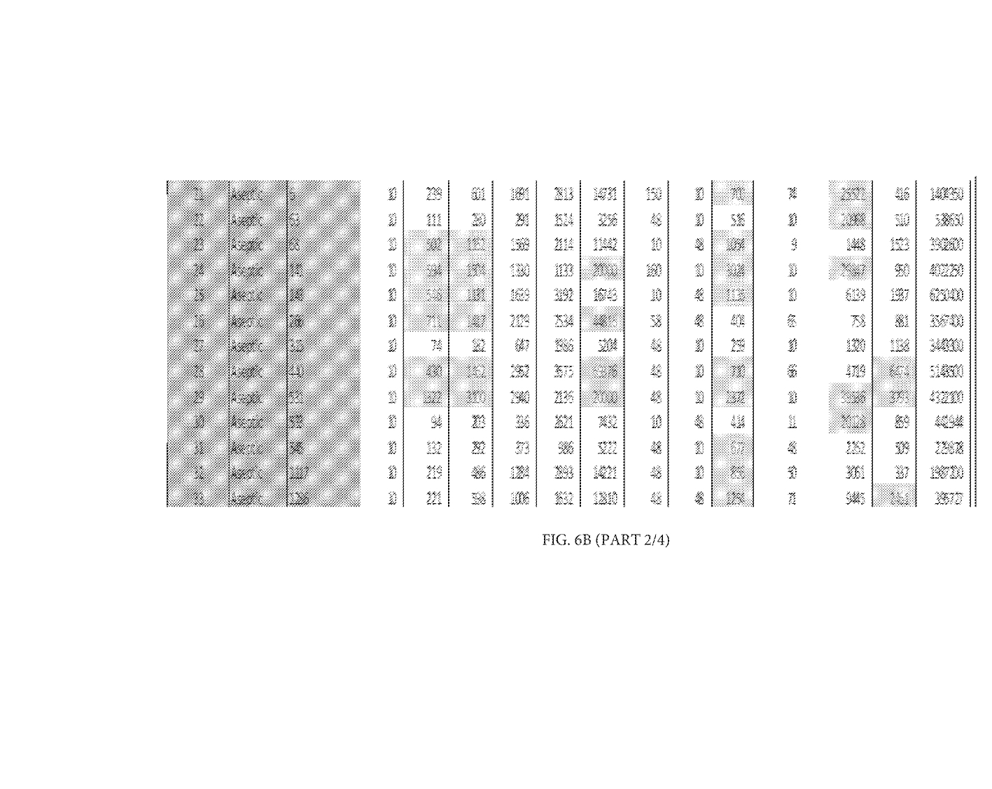

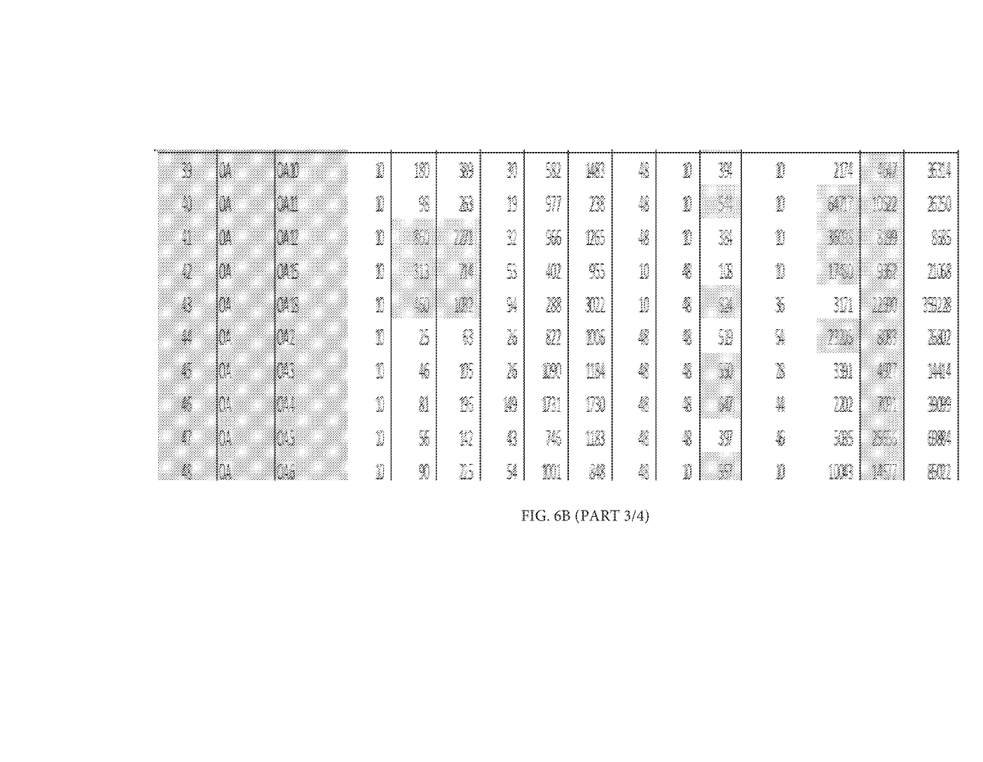

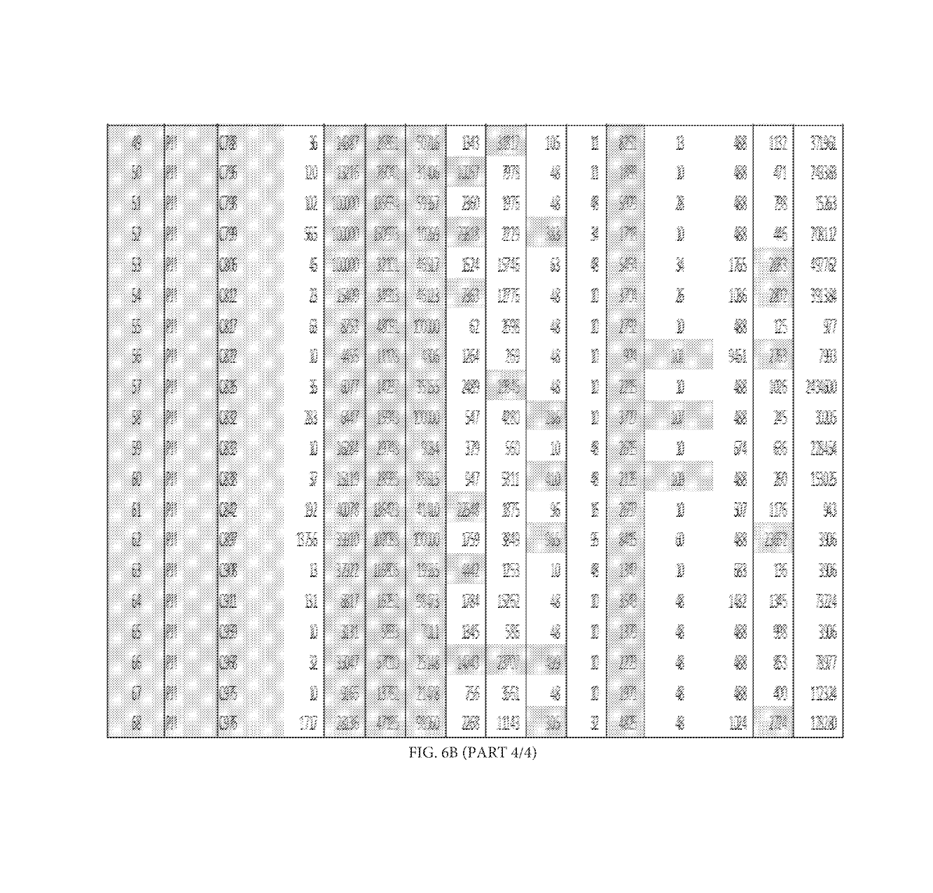

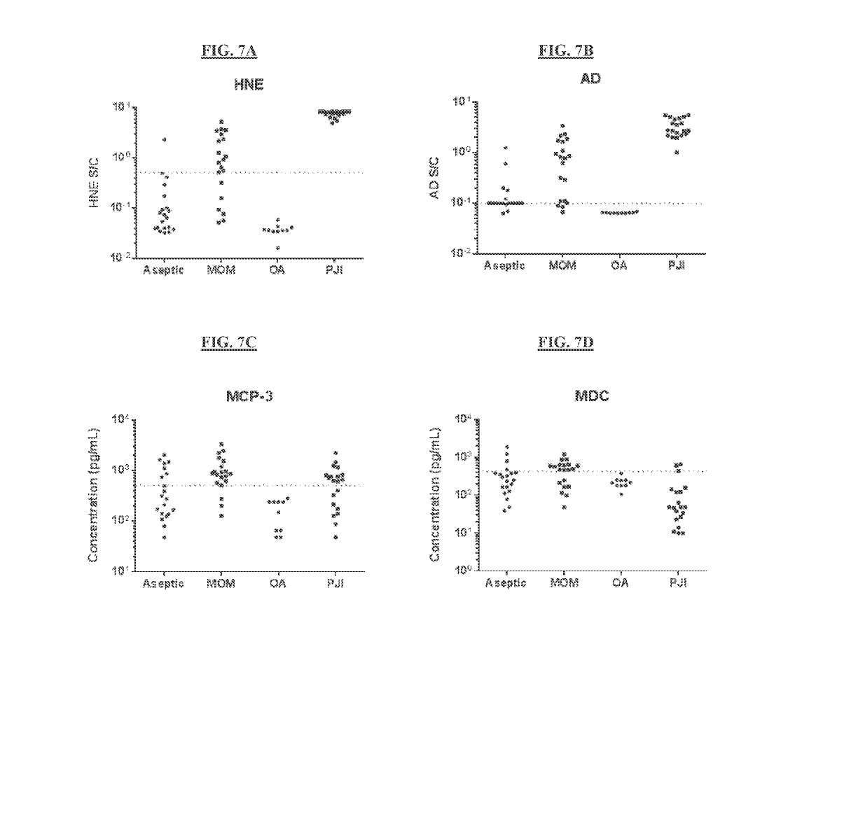

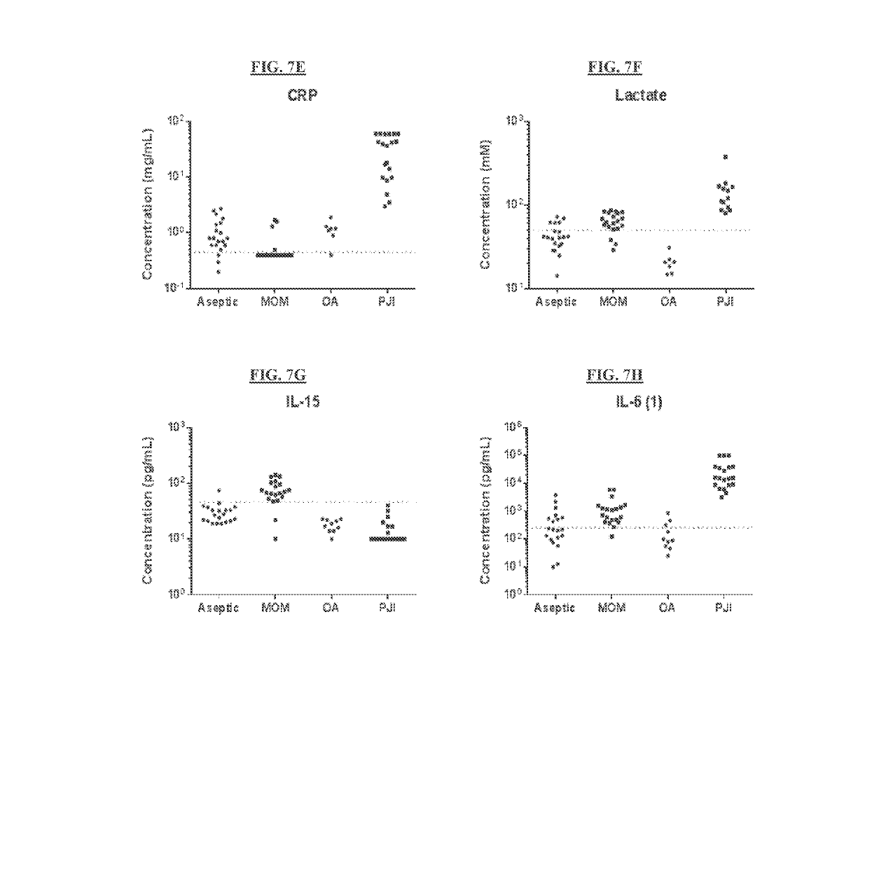

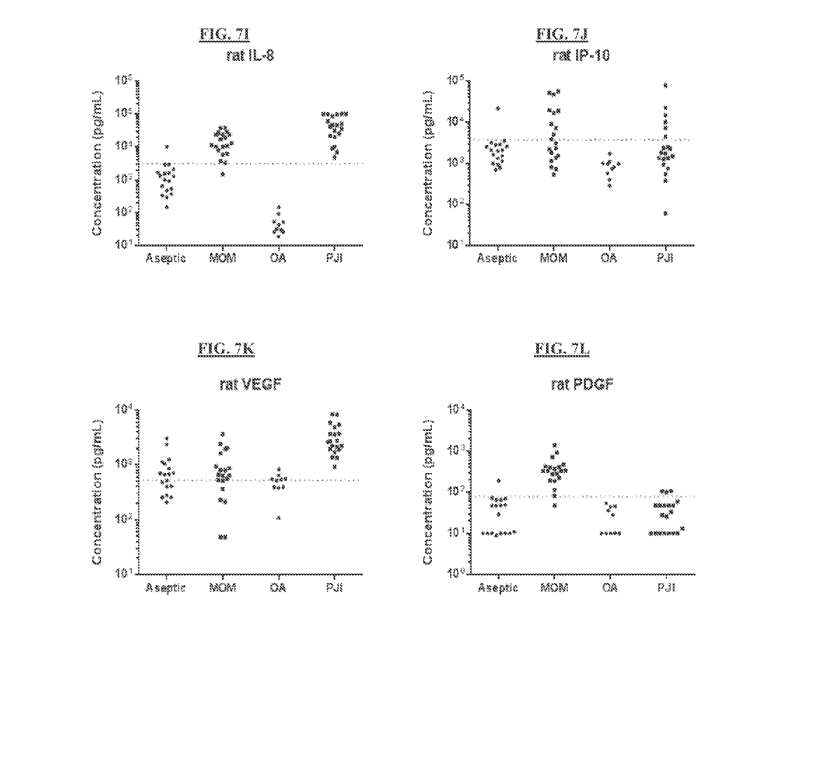

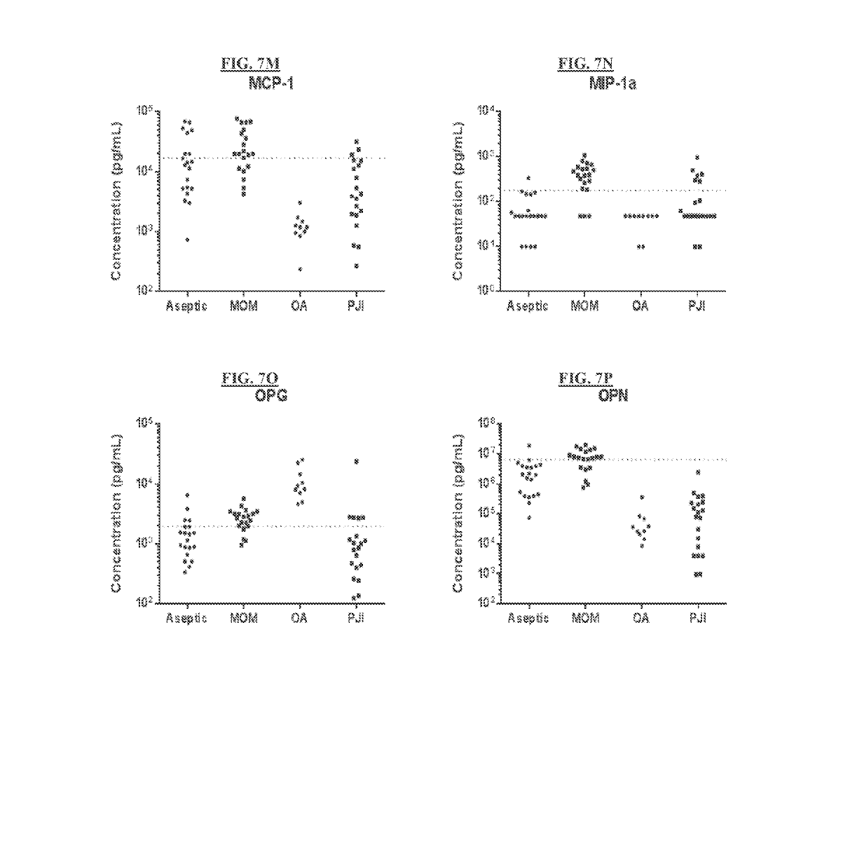

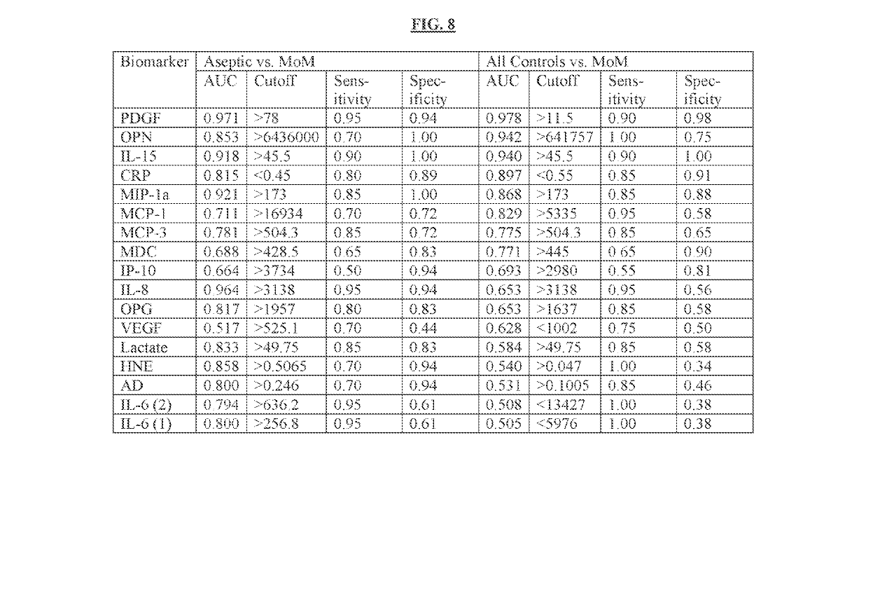

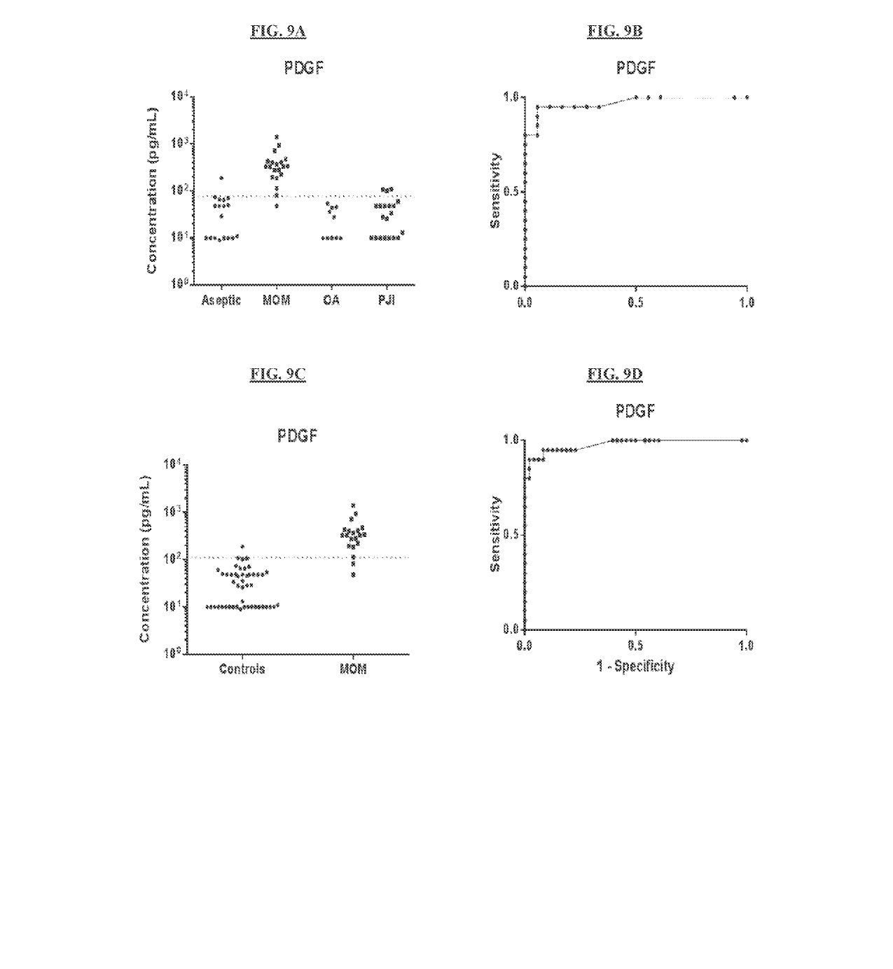

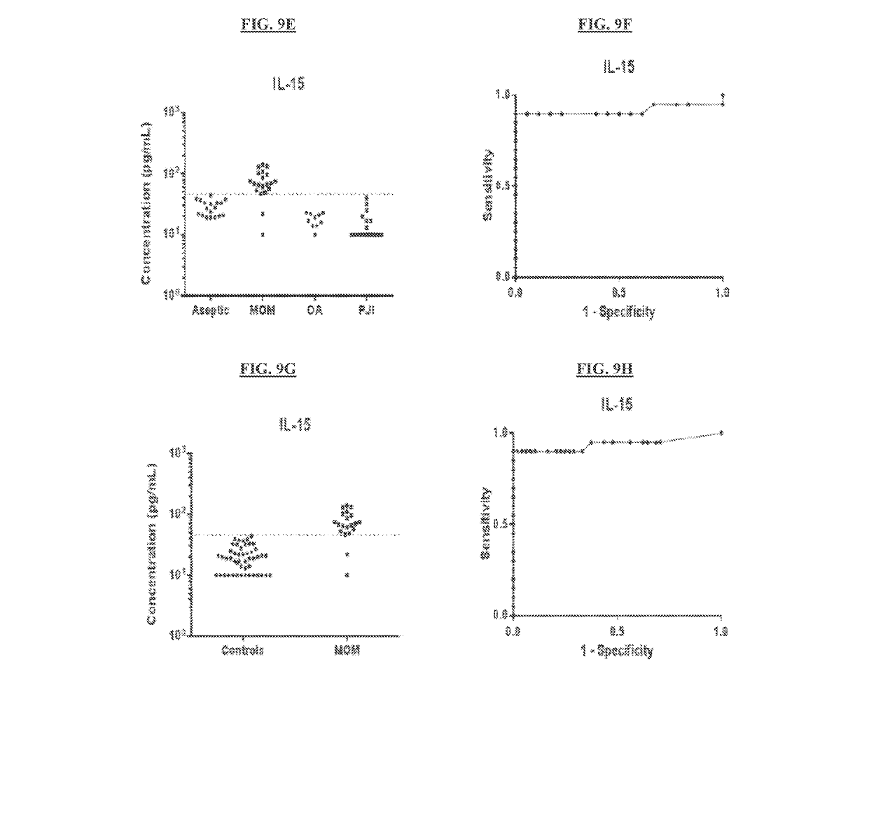

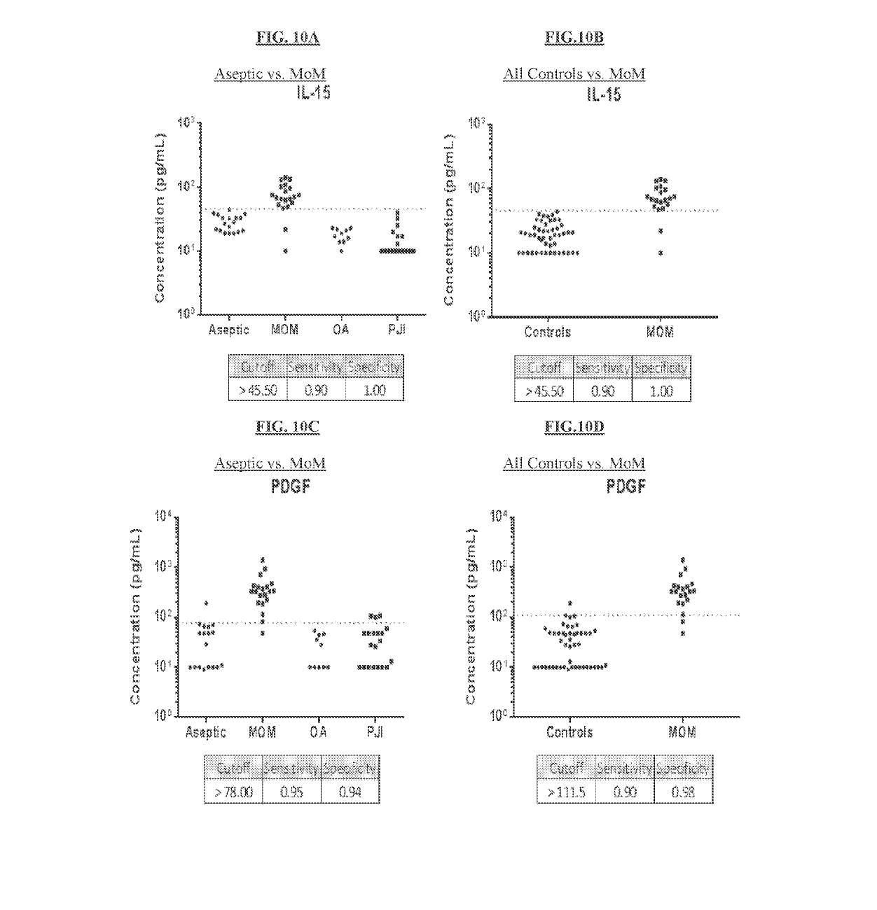

In the secondary screening, 68 individual synovial fluid samples were analyzed for 23 unique human biomarkers. The samples comprised 18 aseptic, 20 MOM, 10 OA and 20 PJI individual patient synovial fluids (FIGS. 5A-5C, 6A(Part 1/4) through 6A(Part 4/4), 6B(Part 1/4) through 6B(Part 4/4), and 7A-7P). Similar to the results in the primary screen, INFγ, TNF and IL-12 were negative with almost all samples tested, as were IL-1α and IL-1β. IL-6 and IL-8 showed high sensitivity for MOM samples but both were elevated in PJI samples as well. Three biomarkers, IL-15, MIP1α and OPN showed 100% specificity using ROC cutoffs calculated from the aseptic (control) and MOM (patient) samples. IL-15 was 100% specific using the cutoff calculated from all control samples (aseptic, OA and PJI) vs. the MOM samples. Two biomarkers, PDGFB and IL-15, had high sensitivity and specificity with both methods of calculating cutoffs (FIGS. 8, 9A-9H, 10A-10D and 11A-11D).

In these studies, most biomarkers were negative in OA samples with the exception of OPG which detected all ten samples. This is in agreement with the literature reporting OPG as a biomarker of OA. OPG was also positive in some MOM samples.

The PJI biomarkers HNE, AD and lactate are not good biomarkers of MOM samples using either the cutoffs determined for PJI or the ROC cutoffs determined herein. In contrast, very low concentrations of the PJI biomarker CRP had sensitivity and specificity of approximately 80-90%. The cutoff for CRP in PJI is >3 mg/mL while the cutoff for diagnosing MOM samples is <0.5 (FIG. 12). These findings suggest a possible role for CRP in an algorithm to differentiate PJI from MOM samples.

### Example 5: Multiplex Biomarkers and Algorithms for Diagnosing ALTR and PJI

Both infection and ALTR are serious conditions that required surgical intervention. The procedure for treating infection can also resolve the potential ALTR due to reaction with the implant. Differentiating between infection (e.g. PJI) and ALTR is a significant factor to be taken in account by the physician and this distinction can be achieved using the diagnostic algorithm of the present invention.

In the algorithm presented herein, the physician needs to rule out infection as a cause of the joint failure. For instance, a population of 100 failing joints may have infections, 10 ALTR, and 70 non-septic/non-ALTR failures.

A multitude of markers are used in combination to achieve the desired clinical sensitivity and specificity to determine the difference between PJI and ALTR in a subject.

The goal of the first screen in the algorithm is to identify the samples with the highest potential to be infected or have a high probability of being aseptic/Non-ALTR. The samples that cannot be classified by the first screen are then evaluated using additional markers that are more definitive for ALTR

An example of a biomarker panel collected in the experiments of the present invention is listed below in Table 2. The two main categories of infected (e.g. PJI) and aseptic/non-ALTR are shown. Additionally, 4 biomarker combinations are shown that can confirm the presence of ALTR. Pos=Positive and Neg=Negative.

TABLE 2
Biomarker Panel

| | Diagnosis | | | | | |
|---|---|---|---|---|---|---|
| | | non-ALTR/ | | | | |
| Biomarker | Infected | non-PJI | ALTR | ALTR | ALTR | ALTR |
| Defensin | Pos | Neg | Pos | Neg | Neg | Neg |
| CRP | Pos | Neg | Neg | Neg | Neg | Neg |
| IL-8 | Pos | Neg | Pos | Pos | Neg | Pos |
| MIP- | Pos | Neg | Pos | Pos | Pos | Neg |
| 1alpha | | | | | | |
| PDGFB | NA | NA | Pos | Pos | Pos | Pos |

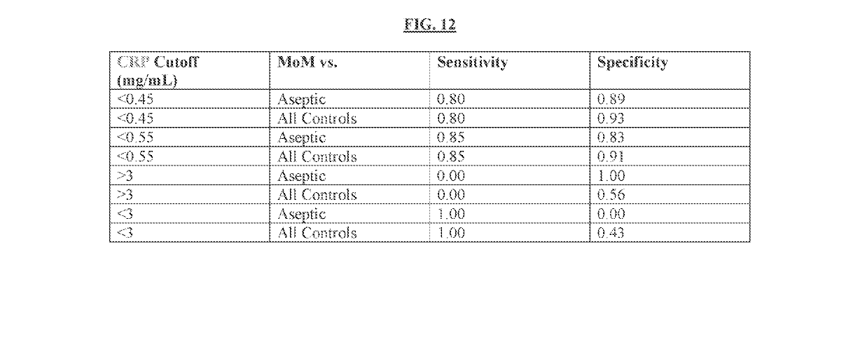

Additionally, each biomarker tested in the present invention exhibited unique patterns of reactivity with individual samples. Some biomarkers exhibited similar patterns with classes of samples but were positive or negative with different members within a class. It is possible to combine biomarkers with overlapping reactivities in a way that a panel of 2 or 3 biomarkers together provides clinical performance superior to individual biomarkers alone. In these studies IL-8 was shown to be elevated in both MOM and PJI samples and therefore not capable of differentiating between the two. However, using a diagnostic algorithm comprised of a first biomarker panel of AD and CRP to remove PJI samples before subsequent analysis with a second biomarker comprised of PDGFB or IL-15 combined with IL-8, MIP1α or OPN enables highly accurate clinical diagnosis of both PJI and ALTR (FIG. 13).

Another example of an algorithm identifying how some biomarkers of the present invention can be used in different combinations to differentiate and diagnose ALTR and PJI is outlined below.
1) Use alpha defensin (AD), CRP, pentraxin3 and IL-8 alone or in any and all combinations to diagnose patients that have either ALTR, PJI or both.
2) Use PDGFB, IL-15, CRP, MIP1a, OPN, and FASL alone or in any and all combinations to diagnose ALTR.
3) Use AD, CRP, pentraxin3 and IL-8 alone or in any and all combinations to diagnose patients that are either PJI or ALTR and remove negative samples from further analysis, then use PDGFB, IL-15, CRP, MIPla, OPN, and FASL alone or in any and all combinations with the ALTR or PJI samples to diagnose ALTR.

Briefly, the use of AD, CRP and IL-8 is of particular interest in first identifying patients that are either ALTR, PJI or both and to remove negative samples from subsequent analysis. Next, the use of PDGFB and IL-15 is relevant to distinguish ALTR from PJI in patients that have ALTR. Many other combinations of the biomarkers of the present invention are also useful and should be considered for diagnosing ALTR and PJI and for distinguishing between the two.

### SUMMARY

PDGFB and IL-15 have been shown to be highly sensitive and specific biomarkers for differentiating MOM ALTR from PJI, aseptic and OA synovial fluid samples. Additional biomarkers have also been identified that when combined together, yield a multiplex panel with superior clinical performance. Algorithms have been developed using multiple biomarkers to diagnose and differentiate PJI and ALTR in patients with orthopedic implants.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for evaluating a subject having an implant, the method comprising:
a. requesting or performing a test to determine whether there is at least one biomarker of adverse local tissue reaction (ALTR) in a bodily fluid sample obtained from the subject; and
b. comparing a level of the at least one biomarker in the sample with a control level, wherein a difference in the level of the at least one biomarker in the sample as compared with the control level is indicative of an ALTR in the subject.

2. The method of claim 1, further comprising:
c. recommending or performing a therapeutic intervention on the subject based on the results of step b.

3. The method of claim 1, wherein the at least one biomarker is selected from the group consisting of Neutrophil defensin 1, C-reactive protein, Growth-regulated alpha protein, Neutrophil elastase, Interleukin 1-alpha, Interleukin 6, Interleukin 8, Interleukin 12-beta, Interleukin 15, C-X-C motif chemokine 10, Lactate, Leptin, Monocyte chemotactic protein 1, Monocyte chemotactic protein 3, C-C motif chemokine 22, Tumor necrosis factor receptor superfamily member 11B, Osteopontin, Platelet-derived growth factor subunit B, Pentraxin-3, Tumor necrosis factor alpha, Vascular endothelial growth factor, Tumor necrosis factor ligand superfamily member 6 and Soluble intercellular adhesion molecule-I.

4. The method of claim 1, wherein the at least one biomarker is selected from the group consisting of Interleukin 15, Platelet-derived growth factor subunit B, Osteopontin, Tumor necrosis factor ligand superfamily member 6, and Soluble intercellular adhesion molecule-I.

5. The method of claim 1, wherein the at least one biomarker is selected from the group consisting of Interleukin 15, Platelet-derived growth factor subunit B, and Osteopontin.

6. The method of claim 1, wherein the at least one biomarker is selected from the group consisting of Interleukin 8, C-reactive protein, Interleukin 12-beta, Interleukin 15, Monocyte chemotactic protein 1, Monocyte chemotactic protein 3, Pentraxin-3, and Tumor necrosis factor ligand superfamily member 6.

7. The method of claim 1, wherein the sample is from the site of the implant, and wherein step b. includes
assessing whether or not T-cells are present at the implant site by assessing the sample for the presence of at least one biomarker of T-cell activity selected from the group consisting of Interleukin 15 and Tumor necrosis factor ligand superfamily member 6; and, the method further comprising:
c. recommending or providing a therapeutic intervention for treating adverse local tissue reaction in the subject when T-cells are detected based on the results of the assessing step.

8. The method of claim 1, wherein the sample is from the site of the implant, and wherein step b. includes assessing whether or not macrophages are present at the implant site by assessing the sample for the presence of at least one biomarker of macrophages selected from the group consisting of Monocyte chemotactic protein 1 and Monocyte chemotactic protein 3; and, the method further comprising:
c. recommending or performing a therapeutic intervention for treating adverse local tissue reaction in the subject when macrophages are detected based on the results of the assessing step.

9. The method of claim 7, wherein the sample is from the site of the implant, and wherein step b. includes assessing the presence of bone growth at the implant site by assessing the sample for the presence of at least one biomarker of bone growth selected from the group consisting of Osteopontin and Platelet-derived growth factor subunit B; and, the method further comprising:
c. recommending or performing a therapeutic intervention for treating adverse local tissue reaction in the subject when bone growth is detected based on the results of the assessing step.

10. The method of claim 7, wherein the sample is from the site of the implant, and wherein step b. includes assessing for the presence of a local inflammatory response by assessing the sample for the presence of at least one biomarker comprising Pentraxin-3, including
comparing the levels of Pentraxin-3 in the sample with a control level; and, the method further comprising:
c. recommending or performing a therapeutic intervention for treating adverse local tissue reaction in the subject if the level of Pentraxin-3 in the sample is greater than the control level.

11. The method of claim 7, wherein the sample is taken from the implant site, and step b. includes assessing the level of at least one biomarker comprising C-reactive protein, including
comparing the level of C-reactive protein in the sample with a control level; and, the method further comprising:
c. recommending or performing a therapeutic intervention for treating adverse local tissue reaction (ALTR) in the subject if the level of C-reactive protein in the sample is less than or equal to the control level and the subject does not have a biomarker that is indicative of infection.

12. (canceled)

13. A method of distinguishing between adverse local tissue reaction (ALTR) and periprosthetic joint infection (PJI) in a subject having an implant, the method comprising:
a. requesting or performing a test on a fluid sample from a joint in the subject, the test comprising an assay for at least one biomarker of ALTR or PJI;
b. comparing the level of the at least one biomarker in the sample with a control level, wherein a difference in the level of the at least one biomarker in the sample as compared with the control level is an indication that the subject has at least one of an ALTR and PJI; and,
c. recommending or providing a therapeutic intervention based on the results of step b.

14. The method of claim 13, wherein step b. includes
analyzing the presence or a level of the at least one biomarker in the sample using an algorithm, wherein the algorithm facilitates differentiation between an ALTR and PJI in the test subject; and, the method further comprising:
d. requesting or performing further testing using at least one additional biomarker, and using the algorithm to confirm the analysis of the at least one biomarker.

15. The method of claim 13 wherein the at least one biomarker comprises one or more of IL-6, IL-8, CRP, PDGF or OPN.

16. The method of claim 15 wherein the at least one biomarker comprises IL-8, OPN, or both.

17. The method of claim 14 wherein the additional biomarker comprises PDGF.

18. The method of claim 13, wherein the therapeutic intervention is a revision surgery.

19. The method of claim 13, wherein the sample comprises at least one selected from the group consisting of blood, serum and synovial fluid.

20. The method of claim 13, wherein the implant is selected from the group consisting of a hip prosthesis, a knee prosthesis, a shoulder prosthesis, an ankle prosthesis, and a wrist prosthesis.

21.-24. (canceled)

25. A kit comprising an antibody or an oligonucleotide probe set configured to specifically bind at least one biomarker selected from the group consisting of Neutrophil defensin 1, C-reactive protein, Growth-regulated alpha protein, Neutrophil elastase, Interleukin 1-alpha, Interleukin 6, Interleukin 8, Interleukin 12-beta, Interleukin 15, C-X-C motif chemokine 10, Lactate, Leptin, Monocyte chemotactic protein 1, Monocyte chemotactic protein 3, CC motif chemokine 22, Tumor necrosis factor receptor superfamily member 11B, Osteopontin, Platelet-derived growth factor subunit B, Pentraxin-3, Tumor necrosis factor alpha, Vascular endothelial growth factor, Tumor necrosis factor ligand superfamily member 6, and Soluble intercellular adhesion molecule-I, and instructions for use thereof, wherein the instructions comprise:
a. measuring the level of the at least one biomarker in a bodily fluid sample from a subject;
b. providing an indication of the presence or absence of ALTR or PJI based on the level of the at least one biomarker; and,
c. providing a recommendation of whether or not the subject should undergo a therapeutic intervention for ALTR or PJI.
